(19) United States
(12) Patent Application Publication
(10) Pub. No.: US 2025/0295694 A1
(43) Pub. Date: Sep. 25, 2025

(54) ADENOSINE DEAMINASE 2 COMPOSITIONS AND METHODS FOR USING SAME

(71) Applicant: Georgia Tech Research Corporation, Atlanta, GA (US)

(72) Inventors: John James Blazeck, Atlanta, GA (US); John Robert Cox, Atlanta, GA (US)

(21) Appl. No.: 18/708,937

(22) PCT Filed: Nov. 11, 2022
(86) PCT No.: PCT/US2022/079692

§ 371 Date: May 9, 2024

Related U.S. Application Data
(60) Provisional application No. 63/278,731, filed on Nov. 12, 2021.

(51) Int. Cl. A61K 35/17; A61K 38/00; A61K 40/11; A61K 40/42; C12N 9/78; C12N 15/86

(52) U.S. Cl. CPC A61K 35/17; A61K 40/11; A61K 40/4244; C12N 9/78; C12N 15/86; A61K 38/00; C07K 2319/30; C12N 2740/15043; C12Y 305/04004

(57) ABSTRACT

The invention is directed to mutants of adenosine deaminase 2 (ADA2) that have improved catalytic activity relative to wildtype ADA2, as well as compositions comprising the mutants and methods of using the mutants to treat various conditions.

### CROSS-REFERENCE TO RELATED APPLICATIONS

This application claims the benefit of U.S. Provisional Application Ser. No. 63/278,731 filed on 12 Nov. 2021, which is incorporated herein by reference in its entirety as if fully set forth below.

### FEDERALLY SPONSORED RESEARCH STATEMENT

This invention was made with government support under grant/award number 1DP2CA280622-01 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Jan. 13, 2023, is named GTRC8854PCT_SL.xml and is 273,461 bytes in size.

### FIELD OF THE DISCLOSURE

The various embodiments of the present disclosure relate generally to mutants of human adenosine deaminase 2 (HsADA2) and more particularly to compositions comprising the mutated HsADA2 enzymes and methods of using the compositions to treat cancer and tumors. In some embodiments, the mutations are located in or near the catalytic site of HsADA2, including the “entry gate” to the catalytic site, and alter the hydrophobicity of the amino acids at specific positions, thus conferring improved catalytic activity relative to the wildtype HsADA2.

### BACKGROUND

Cancers accumulate metabolic byproducts that inhibit immune responses, notably adenosine (ADO). The hypoxic tumor environment stimulates accumulation of ADO from ATP. ADO inhibits T cell, NK cell, and innate immune cell activation and proliferation, and promotes Treg development. High ADO levels limit the efficacy of autologous T cell therapies, inhibit T cell tumoral infiltration, and correlate with poor survival across cancers. As described herein, the inventors have engineered human cells to secrete enzymatic weapons that directly degrade ADO (see FIG. 1). When secreted by autologous T cell therapies, these enzymatic weapons can grant T cells control of their local metabolic environment, protecting them from suppression and invigorating bystander immune cells that had been suppressed by a tumor. Because ADO also inhibits T cell tumoral infiltration, weaponized T cells can also be better able to infiltrate and activate within tumors.

Attempts to prevent the impact of ADO on autologous T cell therapies have been hindered due to redundancies in ADO's syntheses pathways, as both steps of ADO synthesis from ATP are catalyzed by at least six enzymes. Targeting a single synthesis enzyme (e.g., CD73) reduces Ado concentration by only ˜50%, but a 90 to 99% reduction is required to prevent suppression. Efforts to antagonizing Ado receptors (ARs) have been slowed because immune cells express four ARs. The inventors have shown that directly degrading tumoral ADO administering enzymes can help overcome these challenges, though administered enzymes diffuse throughout the body, preventing local impact and requiring consistent dosing.

Adenosine is a potent immunosuppressive metabolite that accumulates in the extracellular space within solid tumors and inhibits the antitumor function of native immune cells responses as well as chimeric antigen receptor (CAR) T cell therapies.

Chimeric antigen receptor (CAR) T cell therapies have demonstrated groundbreaking success in the treatment of blood-based cancers. Unfortunately, CAR-T efficacy against solid tumor indications has been limited, in part due to the immunosuppressive tumor microenvironment (TME). Recently, engineering T cell therapies to be ‘armored’ against the immunosuppressive TME has been shown to increase their antitumor function. To date, most T cell ‘armor’ has been designed to enable their resistance to receptor-mediated or cytokine-mediated immune (i.e., protein-mediated) checkpoints (e.g., secrete αPD-1 scFvs or IL-12). While such approaches are promising, T cells face additional immunosuppressive barriers in the TME, such as the accumulation of inhibitory small molecule metabolites like adenosine.

Adenosine is a ribonucleoside that inhibits the function of a wide variety of immune cells, including T cells. Physiological concentrations of adenosine are in the nanomolar range, but it can accumulate in solid tumors up to concentrations of 100 μM when extracellular ATP is dephosphorylated by a collection of redundant ecto-enzymes (CD39, CD73, TRACP, TNAP, PLAP, etc.). Adenosine has been shown to potently inhibit T cell function by signaling through the adenosine receptors A2AR and A2BR to activate the immunosuppressive cyclic adenosine monophosphate & protein kinase A (cAMP/PKA) pathway. Thus, the A2AR and ecto-enzymes CD39/CD73 have been identified as key clinical targets for small molecule antagonists or monoclonal antibodies with varying degrees of efficacy, in part due to the redundancies in adenosine generation and signaling. In the context of T cell therapies, the CRISPR-Cas9 mediated knockout of the A2AR was recently shown to partially rescue CAR T cell function in solid tumor preclinical models.

There is therefore a need for an improved composition and/or method of inhibiting ADO's effect on the immune system that can target all mechanisms of ADO-induced suppression. It is to such a composition and method that embodiments of the invention are directed.

### BRIEF SUMMARY

The present disclosure relates to mutants of human adenosine deaminase 2 (HsADA2) and more particularly to compositions comprising the mutated HsADA2 enzymes, immune cells comprising the mutated HsADA2 enzymes, and methods of using the compositions and immune cells containing the HsADA2 variants to treat cancer and tumors. In some embodiments, the mutations are located in or near the catalytic site of HsADA2, including the “entry gate” to the catalytic site, and alter the hydrophobicity of the amino acids at specific positions, thus conferring improved catalytic activity relative to the wildtype HsADA2.

In one aspect, the present invention provides a nucleic acid molecule encoding human adenosine deaminase 2 (HsADA2), wherein the HsADA2 has been mutated to have increased catalytic activity relative to a wildtype HsADA2.

In another aspect, the present invention provides an amino acid sequence comprising at least one mutation at one or more amino acid positions of a human adenosine deaminase 2 (HsADA2) enzyme comprising an entry gate to a catalytic site of the HsADA2 enzyme, wherein the at least one mutation confers improved catalytic activity relative to a wildtype HsADA2.

In another aspect, the present invention provides a composition comprising an amino acid sequence comprising at least one mutation at one or more amino acid positions of a human adenosine deaminase 2 (HsADA2) enzyme comprising an entry gate to a catalytic site of the HsADA2 enzyme, wherein the at least one mutation confers improved catalytic activity on the amino acid sequence relative to a wildtype HsADA2 enzyme.

In another aspect, the present invention provides a composition comprising one or more cells, the one or more cells comprising an amino acid sequence comprising at least one mutation at one or more amino acid positions of a human adenosine deaminase 2 (HsADA2) enzyme comprising an entry gate to a catalytic site of the HsADA2 enzyme, wherein the one or more cells express and secrete the amino acid sequence, and wherein the at least one mutation confers improved catalytic activity on the amino acid sequence relative to a wildtype HsADA2 enzyme.

In another aspect, the present invention provides a method of producing at least one cell configured to express a human adenosine deaminase 2 (HsADA2) polypeptide, the method comprising: introducing a nucleic acid molecule encoding the HsADA2 polypeptide into the at least one cell via a viral vector, electroporation, or liposomal mediated introduction; integrating the nucleic acid encoding the HsADA2 polypeptide into the genome of the at least one cell; and culturing the at least one cell in vitro under conditions suitable for expression of the HsADA2 polypeptide and expansion of the at least one cell, wherein the HsADA2 polypeptide comprises at least one mutation at one or more amino acid positions comprising an entry gate to a catalytic site of the HsADA2 polypeptide, wherein the at least one mutation confers improved catalytic activity relative to a wildtype HsADA2 polypeptide, and wherein the nucleic acid molecule encoding the HsADA2 polypeptide comprises a strong promoter and a terminator operably connected to the nucleic acid.

In another aspect, the present invention provides method of treating a cancer or tumor in a subject in need thereof, the method comprising: administering to the subject a therapeutically effective amount of a composition comprising immune cells configured to express a human adenosine deaminase 2 (HsADA2) polypeptide, wherein the HsADA2 polypeptide comprises at least one mutation at one or more amino acid positions comprising an entry gate to a catalytic site of the HsADA2 polypeptide, and wherein the at least one mutation confers improved catalytic activity relative to a wildtype HsADA2 polypeptide.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides a schematic of a workflow for using T cell enzymatic weapons to fight cancer, in accordance with an exemplary embodiment of the present invention.

FIG. 2 provides a schematic of a workflow for CAR T cell weaponization, in accordance with an exemplary embodiment of the present invention.

FIG. 3 provides depiction showing how ADA1 and ADA2 expression was driven by a CMV promoter and preceded by the indicated signal peptide (SP) sequence, in accordance with an exemplary embodiment of the present invention.

FIG. 4 provides representative images of eGFP after SP-ADA1 constructs show high and uniform transfection efficiency (80 to ˜100%), in accordance with an exemplary embodiment of the present invention.

FIG. 5 provides schematic showing how extracellular adenosine degradation rate mediated by ADA1 or ADA2 expression was determined after their transfection into HEK293T cells, in accordance with an exemplary embodiment of the present invention.

FIG. 6 provides Western blots of cell lysate and supernatant for untransfected 293T cells and ADA1/ADA2 transfected cells. GAPDH was used as a housekeeping gene. ** denotes p<0.01 by Tukey HSD test, in accordance with an exemplary embodiment of the present invention.

FIG. 7 provides a schematic of a workflow for screening of the ADA2 library, in accordance with an exemplary embodiment of the present invention.

FIG. 8A provides a schematic showing secretion of ADA2WT and ADA2-R222T/S265A from a human cell line, in accordance with an exemplary embodiment of the present invention.

FIG. 8B provides a schematic showing secretion of ADA2WT and ADA2-R222T/S265A from the Jurkat human T cell line, in accordance with an exemplary embodiment of the present invention.

FIG. 9 provides SDS-PAGE image of purified ADA2 wt and ADA2v20. Numbers represent ladder sizes in kDa. ADA2 wt and ADA2v20 both resolve at about ˜60 kDa (monomer), as expected on a reducing gel, in accordance with an exemplary embodiment of the present invention.

FIG. 10A provides a plot showing HsADA1 and HsADA2 secretion by HEK293T cells, in accordance with an exemplary embodiment of the present invention.

FIG. 10B provides a plot showing extracellular versus intracellular adenosine degradation for HsADA1 and HsADA2 expressing HEK293T cells, in accordance with an exemplary embodiment of the present invention.

FIG. 11A provides an Alphafold structural prediction of ADA2 wt (mesh) and S. peregrina ADA2 (surface) without docked ligand colored for hydrophobicity using PyMOL color h function, in accordance with an exemplary embodiment of the present invention.

FIG. 11B provides a PDB 3lgg ribbon depiction of the ADA2 hydrophilic residues selected for mutagenesis at the entry gate of the active site, in accordance with an exemplary embodiment of the present invention.

FIG. 12A provides a plot of library performance by relative adenosine degradation rate (subtracting the background adenosine degradation of non-transfected 293T control). Each point represents an ADA2 variant within the library, in accordance with an exemplary embodiment of the present invention.

FIG. 12B provides a plot of The top 20 candidates from FIG. 12A, as adenosine degradation rate normalized by GLuc were rescreened in replicate, n=6. Bars represent the mean fold change in adenosine degradation rate compared to wildtype ADA2 and error bars represent SEM, in accordance with an exemplary embodiment of the present invention.

FIG. 12C provides a plot of ADA2v20 outperforms ADA1 and ADA2 wt over a range of adenosine concentrations, in accordance with an exemplary embodiment of the present invention.

FIG. 13A provides a plot of adenosine degradation rate by ADA2 wt, ADA1, and ADA2v20 in HEK293T cells 48 hours post transfection, in accordance with an exemplary embodiment of the present invention.

FIG. 13B provides a plot of adenosine degradation rate mediated by the secreted ADA2v20 in stably transduced Jurkat T cells subtracted from media background, in accordance with an exemplary embodiment of the present invention.

FIG. 14A provides a plot a plot showing adenosine degradation rates, in accordance with an exemplary embodiment of the present invention.

FIG. 14B provides a plot showing adenosine degradation rates for various T cells, in accordance with an exemplary embodiment of the present invention.

FIG. 15 provides a plot showing fold channel in adenosine degradation rates for various mutants, in accordance with an exemplary embodiment of the present invention.

FIG. 16 provides a plot showing adenosine degradation rates for various mutants, in accordance with an exemplary embodiment of the present invention.

FIG. 17 provides a plot showing adenosine degradation rates for various mutants, in accordance with an exemplary embodiment of the present invention.

FIG. 18 provides a plot showing adenosine degradation rates for various mutants, in accordance with an exemplary embodiment of the present invention.

FIG. 19 provides a plot showing adenosine degradation rates for various mutants, in accordance with an exemplary embodiment of the present invention.

FIG. 20 provides a plot showing adenosine degradation rates for various mutants, in accordance with an exemplary embodiment of the present invention.

FIG. 21 provides Western blots of cell lysate and supernatant for untransfected 293T cells and ADA1/ADA2 transfected cells. GAPDH was used as a housekeeping gene. ** denotes p<0.01 by Tukey HSD test, in accordance with an exemplary embodiment of the present invention.

FIG. 22 provides a plot showing lysate adenosine degradation rate versus supernatant degradation rate, in accordance with an exemplary embodiment of the present invention.

FIG. 23A provides a plot showing percent change in supernatant adenosine degradation rates for various mutants, in accordance with an exemplary embodiment of the present invention.

FIG. 23B provides a plot showing percent change in supernatant adenosine degradation rates for various mutants, in accordance with an exemplary embodiment of the present invention.

FIG. 24A provides a plot showing transfection efficiency versus amount of DNA, in accordance with an exemplary embodiment of the present invention.

FIG. 24B provides a plot showing transfection efficiency versus amount of DNA, in accordance with an exemplary embodiment of the present invention.

FIG. 25 provides a plot showing adenosine degradation rate versus Gluc activity, in accordance with an exemplary embodiment of the present invention.

FIG. 26 provides a plot showing relative adenosine degradation rate versus adenosine concentration for various mutants, in accordance with an exemplary embodiment of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint(s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, human and veterinary animals. In a preferred embodiment, the subject is human.

As used herein, the term “combination” of a composition comprising a mutated ADA2 and at least a second pharmaceutically active ingredient means at least two, but any desired combination of compounds can be delivered simultaneously or sequentially (e.g., within a 24-hour period). It is contemplated that when used to treat various diseases, the compositions and methods of the present invention can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

### Compositions of the Invention

According to some aspects, the present invention provides mutated HsADA2 enzymes and nucleic acids encoding those enzymes, as well as compositions comprising the mutated HsADA2 enzymes, preferably present in therapeutically effective amounts.

Near the active site of HsADA2, there exists an entry gate that is considerably more hydrophilic than its phylogenetic homologs at and around ˜10 Å of the residues S179, E182, T183, S189, H193, A221, R222, L224, S265, D266, H267, S269, and H301. This structural difference may be responsible for HsADA2's high KM (2.2 mM) compared to other organisms (S. peregrina, K_{M}=15 μM). The inventors have shown enormous plasticity and mutational compatibility with hydrophobic (or other) residues at positions E182, A221, R222, L224, S265, D266, S269, H267, and H301, which makes targeting this entry-gate region a generalizable engineering strategy for improving the catalytic efficiency of HsADA2, particularly when screening combinations of potential mutations for enhanced HsADA2 activity. In all these residues consist of the linear amino acids V176-V197, M217-E228, 1262-A273, and F296-G305.

The numbering used herein is in the convention of crystal structures PDB: 3lgg and 3lgd and Zavialov et al. (2010). The numbering begins from the signal peptide (SP)-cleaved version of HsADA2. Positions 1 onward are shown below for clarity. This the convention is for the ‘mature’ HsADA2 protein, in which the secretory signal peptide with the sequence “MLVDGPSERPALCFLLLAVAMSFFGS” (SEQ ID NO: 102) has been removed. Therefore, instead of residue number 1 being M, residue #1 is the residue after MLVDGPSERPALCFLLLAVAMSFFGS (SEQ ID NO: 102).

To improve the catalytic activity of the HsADA2 enzyme, the inventors mutated one or more amino acid positions at or near an entry gate to a catalytic site of the enzyme, and optionally one or more amino acid positions within the catalytic site. These mutations altered the hydrophobicity of the amino acids at these positions, thus improving the catalytic activity of the HsADA2 variants.

The residues described herein include positions V176-V197, M217-E228, 1262-A273, and/or F296-G305. All positions described herein are relative to wildtype HsADA2 found in SEQ ID NO: 2. The mutated ADA2 enzymes can comprise more than one mutation in these positions; for example, a mutated HsADA2 enzyme can comprise two mutations or three mutations in these positions. Preferred positions include S179, E182, T183, S189, H193, A221, R222, L224, S265, D266, H267, S269, and/or H301.

Preferred substitutions include M, G, or V at position E182; S or T at position A221; Q, S, or T at position R222; S or P at position L224; N, T, or A at position S265; P or A at position D266; V or I at position H267; A, G, or M at position S269; and/or Q at position H301.

In some embodiments, the HsADA2 variant includes at least one mutation in positions R222, L224, S265, and/or H301, preferably comprising substitutions R222T, L224S, S265A, and/or H301Q.

In some embodiments, the HsADA2 variant can include at least one additional mutation in one or more of positions L87, 190, and/or 192. Preferred substitutions include V or F at L87; F at 190; and/or F at 192.

In an aspect, the invention provides a nucleic acid molecule encoding human adenosine deaminase 2 (HsADA2), wherein the HsADA2 has been mutated to have increased catalytic activity relative to a wildtype HsADA2 by altering positions in or near the catalytic site or the gate to the catalytic site of the enzyme, and that result in an alteration in the hydrophobicity at those positions. The mutations can be any of the mutations described herein. For example, the HsADA2 nucleic acid includes one or more mutations at positions S179, E182, T183, S189, H193, A221, R222, L224, S265, D266, H267, S269, and/or H301, optionally in combination with mutations at one or more of positions L87, 190, and/or 192. Preferred substitutions include M, G, or V at position E182; S or T at position A221; Q, S, or T at position R222; S or P at position L224; N, T, or A at position S265; P or A at position D266; V or I at position H267; A, G, or M at position S269; and/or Q at position H301; optionally in combination with V or F at L87; F at 190; and/or F at 192. The nucleic acid molecule can comprise a nucleotide sequence as set forth in SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 87, 89, 91, 93, 95, 97, and 99.

The nucleic acid molecule encoding the HsADA2 variant can be present in a vector (e.g., a viral vector or a plasmid) or in an expression cassette. The vector or expression cassette can be present in a cell, such as a prokaryotic cell or eukaryotic cell. The vector or expression cassette may be permanently or transiently integrated into the host cell genome, or may be maintained extrachromasomally by suitable methods such as selection pressure. The vector or expression cassette can include a strong promoter operably linked to the nucleic acid molecule encoding the HsADA2 variant. The promoter can be inducible or constitutive. Nonlimiting examples of prokaryotic cells include cells suitable for expression of heterologous proteins, such as for example and not limitation, Escherichia coli. Nonlimiting examples of eukaryotic cells include mammalian cells, such as immune cells and non-immune cells. Nonlimiting examples of immune cells include T cells, CAR T cells, B cells, natural killer (NK) cells, and neutrophils. Nonlimiting examples of non-immune cells can include cell lines that are suitable for expression of heterologous proteins, such as for example and not limitation, Chinese hamster ovary (CHO) cells, HEK293T Cells, and Expi293T cells.

In an aspect, the invention provides a polypeptide encoding human adenosine deaminase 2 (HsADA2), wherein the HsADA2 has been mutated to have increased catalytic activity relative to a wildtype HsADA2 by altering positions in or near the catalytic site or the gate to the catalytic site of the enzyme, and that result in an alteration in the hydrophobicity at those positions. The mutations can be any of the mutations described herein. For example, the HsADA2 nucleic acid includes one or more mutations at positions S179, E182, T183, S189, H193, A221, R222, L224, S265, D266, H267, S269, and/or H301, optionally in combination with mutations at one or more of positions L87, 190, and/or 192. Preferred substitutions include M, G, or V at position E182; S or T at position A221; Q, S, or T at position R222; S or P at position L224; N, T, or A at position S265; P or A at position D266; V or I at position H267; A, G, or M at position S269; and/or Q at position H301; optionally in combination with V or F at L87; F at 190; and/or F at 192. The polypeptide can comprise an amino acid sequence as set forth in SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 88, 90, 92, 94, 96, 98, and 100.

In an embodiment, the mutated HsADA2 polypeptide can be modified to increase its secretory properties, half-life, solubility, and/or ability to interact with certain cancers and/or tumors (e.g., solid tumors by way of adding a domain that binds to or interacts with collagen). In an embodiment, the mutated HsADA2 polypeptide can be operably linked to a heterologous secretory signal peptide (SSP), such as for example and not limitation, secretory signal peptides from PROS, RNase4, CLM9, CC122, GRAB, IgKCm, Albumin (HSA), CATE, IL-19, CD177, ADA2, CYXL, and/or IL-2. In an embodiment, the mutated HsADA2 polypeptide can be operably linked to an antibody fragment, such as for example and not limitation, an Fc portion, to create a peptibody. In an embodiment, the HsADA2 polypeptide can be PEGylated at its N-terminus and/or C-terminus. The mutated HsADA2 polypeptide can also be operably linked to an scFv portion of an antibody. In an example, the scFv portion can be from a collagen-associated antibody. The mutated HsADA2 polypeptide can be operably linked to a collagen-binding peptide or a collagen-like peptide. All of these modifications are encompassed within the terms “HsADA2 variants” or “mutated HsADA2 enzymes”.

In another aspect, the invention provides a composition comprising one or more HsADA2 variants as described herein (referred to herein as HsADA2-containing compositions). The HsADA2 variant is present in a therapeutically effective amount. The HsADA2-containing composition can further comprise additional ingredients, such as for example and not limitation, a pharmaceutically acceptable excipient and/or carrier.

The HsADA2-containing composition can be formulated for administration by any appropriate route, such as for example and not limitation, intratumoral, peritumoral, intradermal, subcutaneous, intravenous, or intraperitoneal administration. Suitable excipients and/or carriers can be chosen based on the route of administration.

In another aspect, the invention provides a composition comprising cells that comprise one or more HsADA2 variants as described herein. The cells can express and secrete the HsADA2 variants. In an embodiment, the cells are mammalian cells such as an immune cell, a cancer cell, a tumor cell, a healthy non-immune cell, a T cell, a CAR T cell, a natural killer (NK) cell, a B cell, and/or a neutrophil. In another embodiment, the cells are immune cells, preferably T cells, CAR T cells, natural killer (NK) cells, B cells, and/or neutrophils. The composition is administered to a subject in a therapeutically effective amount, by any appropriate route, such as for example and not limitation, intratumoral, peritumoral, subcutaneous, intradermal, intravenous, or intraperitoneal administration. Suitable excipients and/or carriers can be chosen based on the route of administration. The composition can be administered in combination with one or more cancer immunotherapies as described herein. It is also contemplated that the cells can be further modified to express and secrete additional immunogenic proteins, or the cells can be further modified to have inducible control over cell functions.

### Combination Therapies

Any of the HsADA2-containing compositions (polypeptides or cells expressing and secreting the HsADA2 variants) disclosed herein can be used in combination with cancer immunotherapy regimens, a non-limiting list of which is provided herein. The HsADA2-containing compositions can either be co-administered with engineered T cells or the engineered T cells could be engineered to simultaneously express and secrete these proteins. The following are examples of possible cancer immunotherapy regimens. Antibody therapies can include: aPD-1, aPD-L1, aCTLA-4, aLAG3, aTIM3, and aHER-2. Bispecific T cell engager (BiTE) Therapies can include aCD3e/aCD19, aCD3e/aCD20, aCD3e/aCD33, aCD3e/FLT3, aCD3e/aHER2, aCD3e/aPSMA, aCD3e/EGFRvIII, aCD3e/DLL3, aCD3e/MUC17, and aCD3e/CLDN18.2. Cytokine therapies can include Neo-2/15, IL-2, IL-7, IL-12, IL-15 (or adaptations, e.g., LT-803), IL-18, IL-21, TNFa, GM-CSF, IFNalpha, and IFNgamma.

The HsADA2 variants described herein, optionally in combination with a cancer immunotherapy as described herein, are useful for treating a condition such as a cancer or a tumor. The HsADA2 variants are present in therapeutically effective amounts, optionally in combination with a pharmaceutically acceptable excipient and/or carrier.

### Methods of Making the HsADA2 Variants

In an aspect, the invention provides a method of producing any of the HsADA2 variants described herein. The method includes introducing a nucleic acid molecule comprising a nucleotide sequence encoding any of the HsADA2 variants as described herein into a cell via a vector or viral vector, electroporation, or liposome-mediated introduction, integrating the nucleotide sequence encoding the HsADA2 variant(s) into the host cell genome, and culturing the cell in vitro under conditions suitable for expression and secretion of the HsADA2 variant(s). Preferably, the HsADA2 variant is expressed from a strong promoter (optionally an inducible promoter) and is followed by a terminator.

Suitable host cells include, but are not limited to, mammalian cells such as an immune cell, a cancer cell, a tumor cell, a healthy non-immune cell, a T cell, a CAR T cell, a natural killer (NK) cell, a B cell, and/or a neutrophil.

### Methods of Treatment Using the HsADA2 Variants

In an aspect, the invention provides a method of treating a disease or condition in a subject in need thereof by administering a therapeutically effective amount of a composition comprising cells comprising a HsADA2 variant and configured to express and secrete the HsADA2 variant, as described herein. The cells can be an immune cell, a cancer cell, a tumor cell, a healthy non-immune cell, a T cell, a CAR T cell, an NK cell, a B cell, and/or a neutrophil. The composition can further comprise a pharmaceutically acceptable excipient and/or carrier, and can be formulated for any method of administration, such as intratumoral, peritumoral, subcutaneous, intravenous, or intraperitoneal administration. The composition can also be administered with one or more additional cancer immunotherapies. It is also contemplated that the cell expressing the HsADA2 variant can be further modified to express and secrete additional immunogenic proteins, or that the cell can be further modified to have inducible control over cell functions.

The following examples further illustrate aspects of the present disclosure. However, they are in no way a limitation of the teachings or disclosure of the present disclosure as set forth herein.

### EXAMPLES

### Example 1: Engineering CAR T Cells to Overcome Adenosine-Mediated Immune Suppression

Adenosine deaminase (ADA) enzymes can irreversibly deaminate adenosine into inosine, a metabolite that binds A2AR with ˜1000× weaker affinity and can promote TH1 cell differentiation during activation. Additionally, it was recently shown that inosine (but not adenosine) can serve as an alternative carbon source for glucose-restricted T cells in tumors, such that bolus injections of inosine could augment their anti-tumor efficacy in mice. Humans have two ADA isoforms: a primarily intracellular variant, ADA1, that is ubiquitously expressed and a secreted variant, ADA2, that is found in serum and mostly expressed in myeloid cells. ADA1 is considerably more active than ADA2 as it has a higher k_{cat} (˜190 s⁻¹ compared to 45 s⁻¹) and nearly 100-fold lower K_{M} (26 μM compared to 2 mM). Other than its role in purine salvage, ADA1 moonlights as a positive regulator of lymphocyte activation, adhesion, and differentiation via interaction with surface protein CD26. ADA2 may have a role in clearing elevated extracellular adenosine concentrations, but has other roles in monocyte-macrophage differentiation and CD4+ T cell proliferation, both of which are largely independent of its adenosine deaminase activity.

In this work, the inventors developed a way to engineer human cells, particularly T cells, to allow them to remodel the tumoral extracellular metabolic environment and eliminate the immunosuppressive adenosine metabolite. The inventors showed that despite being largely intracellular restricted, constitutive ADA1 expression in HEK293 cells results in superior extracellular adenosine deamination activity than the naturally secreted ADA2 enzyme, due to ADA1's higher activity. Surprisingly, the inventors were unable to improve ADA1 secretion, despite the introduction of an either canonical or computationally predicted N-terminal signal peptides or Fc-fusions. Therefore, to improve the ability of an engineered to cell to degrade extracellular adenosine, the inventors engineered human ADA2 variants with more favorable kinetic properties. By generating a library of ADA2 variants based on a phylogenetic analysis of eukaryotic homologs, and developing a complementary reverse transfection-based high throughput screen for extracellular ADA activity, the inventors were able to isolate an improved ADA2 variant with a ˜7-fold reduction in KM (˜2 mM to ˜0.3 mM) that is more active at tumoral adenosine concentrations. Additionally, the inventors demonstrated that both Jurkat and primary human T cells can secret the enzyme. These results suggest that engineering T cells with ADA2 (or a variant thereof) could be a strategy to directly deplete adenosine in solid tumors to improve T cell and bystander immune cell function. In future work, ADA2-engineered T cells will be tested in a suitable preclinical tumor model to determine if they rescue adenosine-mediated T cell dysfunction in vivo.

Materials and Methods: cell lines and cultivation: HEK293T cells were maintained in GlutaMAX and 10% Fetal Bovine Serum (FBS) from Gibco™. Jurkat cells were cultured in RPMI-1640 (Gibco™) supplemented with 10% FBS. All cell lines used in this study were tested for mycoplasma and verified negative.

Relating to plasmid and ADA2 library construction: ADA1 and ADA2 human code determining sequences (CDS's) were ordered as double stranded gene fragments (with 6×His tags to ease detection) from Twist Bioscience. All ADA2 and ADA1 T2A-Gluc constructs were amplified via PCR and inserted into a pcDNA3 backbone via Gibson Assembly. The Gluc gene was a generous gift from Dr. Gabe Kwong (BMED, Georgia Tech). ADA2 wt and ADA2v20 expression constructs for protein purification were PCR′d and assembled into a pcDNA3.1 backbone without a T2A Gluc sequence via Gibson assembly. All lentiviral constructs were constructed via Gibson Assembly and inserted into a pLeGO-C backbone (Addgene #27348) digested with XbaI and EcoRI (New England Biolabs®).

For the ADA2 variant library construction, a base construct containing Esp3I sites upstream of the T2A-Gluc sequence was first constructed from a pcDNA3 backbone via Gibson Assembly. ADA2 mutations were introduced by using degenerate primers that, following PCR, resulted in eight ADA2 fragments that harbored the desired mutations. Next, the fragments were added to the same PCR reaction at equimolar concentrations (normalized by mutations per fragment) and amplified without primers via overlap extension PCR. A final amplification of the re-assembled ADA2 variant library amplicon followed that also added Esp3I overhangs for ligation into the final backbone construct. A primer table can be found in Table 1.

Using standard electroporation protocols, lentiviral plasmids were transformed into NEB® Stable E. coli, while all plasmids with pcDNA backbones were transformed into E. coli strain NEB® 10β 34. All plasmids were sequence confirmed via Sanger sequencing prior to use, and plasmids used for transfection were maxiprepped with PureLink™ HiPure Plasmid Maxiprep Kit (K210007).

For adenosine kinetic assays in supernatant and cell lysates: for adenosine kinetic assay experiments from cell supernatants, HEK293T cells were transiently transfected in 12 well plates with lug of plasmid DNA with TransIT-LT1 transfection reagent (MirusBio, MIR 2305) according to the manufacturer's instructions. 24 hours post transfection, the media was changed to serum free media. Another 24 hours later, the cultured media was taken and spun at 1000×g for 5 minutes to pellet detached cells and 1 mL of supernatant harvested. Adhered cells were washed once with phosphate buffered saline (PBS) −/− pH=7.4 and lysed with 300 μL M-PER™ protein extraction reagent (Thermo Scientific™ #78501) supplemented with Halt™ protease inhibitor cocktail (Thermo Scientific™ #78442) for 5 minutes with gentle agitation. Lysed cells were centrifuged at 14000×g for 5 minutes and the soluble proteins harvested. 40 μL of supernatant or lysate sample was pipetted into UV-Star® 96 well plates (VWR). 160 μL of PBS solution with indicated adenosine concentrations were added to each well and the absorbance a 260 nm was tracked with a SynergyHTX microplate reader for 30 minutes. Adenosine degradation rates were obtained by analyzing the initial linear region of the resulting slope and adjusting for in-plate dilutions. Lysate degradation rates were divided by 10/3 to account for volume equivalences with supernatant for comparison. Standard curves were generated with serum free cell culture media.

For Jurkat cell experiments, 10⁶ cells/mL were seeded in 12 well plates (1 mL culture volume). 48 hours post seeding, cells were spun at 500×g for 5 minutes and supernatant harvested. In-plate kinetic analysis for Jurkat cells was the same as described HEK293T cells above, but with the proper cell culture media (RPMI-1640 and 10% FBS) for standard curves. For primary T cell experiments, 10⁶ cells/mL were seeded in 96 well plates and processed the same as previously described.

Relating to SDS-PAGE & Western Blot: For western blot experiments, HEK293T cells were transfected with the indicated DNA construct in 12 well plates as previously described. 24 hours post transfection, media was changed to serum free media. After another 24 hours, supernatant was sampled and supplemented with Halt™ protease inhibitors. Cells were washed once with PBS −/− and lysed with M-PER™ protein extraction reagent (supplemented with Halt™ protease inhibitor cocktail) for 5 minutes with gentle agitation. Lysed cells were centrifuged at 14000×g for 5 minutes and the soluble proteins harvested and stored at −80° C. until blotting. Protein concentrations of supernatants were assessed by Bradford

Assay (Bio-Rad). For lysates, 3 μg of protein and for supernatants lug of protein were denatured with Bond-Breaker™ TECP (Thermo Scientific, #77720) and 2× Laemmli sample buffer (Bio-rad, #1610737) according to the manufacturer's instruction loaded into each well of a NuPAGE™ 4-12% Bis-Tris gel (Invitrogen™, #NP0322BOX). The SDS-PAGE gel was run on an Invitrogen™ Mini Gel Tank at 200V for 20 minutes in 1×MES buffer (Invitrogen™ NP0002). PVDF membranes (Thermo Scientific™, #88520) were activated with methanol (Sigma-Aldrich) for 5 minutes, then proteins transferred to the membrane in Bolt™ Transfer Buffer (Invitrogen™, #BT00061) supplemented with 10% methanol and 1:1000 NuPAGE™ antioxidants (NP0005) at 30V for 90 minutes. The membrane was then washed with PBS −/− with 0.1% Tween 20 (Sigma Aldrich, #P9416-100ML) three times for 5 minutes, then blocked for an hour at room temperature with Odyssey blocking buffer (LI-COR, #927-70001). After blocking, the membrane was washed 3× for 5 minutes with PBS & Tween again, then stained with 1:250 diluted primary antibodies (R&D Systems, mouse α-His: MAB050-SP, rabbit α-GAPDH: 2275-PC-020) in blocking buffer with 0.02% Tween 20 overnight at 4° C. The following morning, the membranes were washed 3× for 5 minutes with PBS & 0.1% Tween, then secondary antibodies (LI-COR, goat α-mouse IgG: #926-32210, goat α-rabbit: #926-68071) were added at 1:10,000 dilutions in blocking buffer with 0.02% Tween. After another 3× wash step, the membrane was imaged on an Odyssey® DLx imager.

For library screening, ADA2 constructs were tested through HEK293T reverse transfection. In brief, DNA colony picks were miniprepped (Qiagen) and normalized to a concentration of 100 ng/μL. 400 ng of each DNA construct was pipetted into a flat bottom TC treated 96 well plate. Then, prediluted TransIT-LT1 and Opti-MEM solution was added to each well according to the supplier's reverse transfection protocol. Finally, 5×10⁴ HEK293T cells were pipetted into each well and incubated. 48 hours post transfection, 40 μL of supernatant was transferred to UV-Star® 96 well plates (VWR) for adenosine kinetic assays. 20 μL of supernatant was reserved for Gaussia luciferase assays with the Pierce™ Gaussia Luciferase Assay (Thermo Scientific #16161) in white 96 well plates (VWR), according to the manufacturer's instructions.

For ADA2 protein Expression & Purification: 4×10⁶ HEK293T cells were seeded into a 10 cm dish. The next day, 10 μg of pcDNA3.1 vectors coding for ADA2 wt or ADA2v20 were transfected with TransIT-LT1 according to the manufacturer's instruction. 24 hours post transfection, the media was vacuumed off and replaced with serum free GlutaMAX. 24 hours after the media change, the supernatant was spun at 1000×g for 5 minutes, then filtered through a PES 0.2 μm filter (VWR) and supplemented with Halt protease inhibitor cocktail. Next, the supernatant was diluted 1:2 with PBS −/− pH=7.4 and transferred to a superloop. An AKTA Pure 25 fixated with a HisTrap Excel Ni-Sepharose (GE, GE17-3712-06) 1 mL column encased in a 4° C. refrigerator was equilibrated with 5 column volumes (CV's) of equilibration buffer (20 mM sodium phosphate, pH=7.4, 300 mM NaCl). Next, the superloop was loaded (1 mL/min), washed with 5CVs of wash buffer (20 mM sodium phosphate, pH=7.4, 300 mM NaCl, 5 mM imidazole). Bound proteins were eluted (elution buffer, 20 mM sodium phosphate pH=7.4, 300 mM NaCl, 500 mM imidazole) with a linear gradient (0-100% wash buffer elution buffer) up to 5CVs. 1 mL fractions containing elution peaks were pooled and desalted with 5CVs of UPW (Milli-Q), 5CVs of PBS −/− pH=7.4, then the samples were applied and collected after another 2 mL PBS −/− pH=7.4 wash. Finally, the cleaned and diluted samples were transferred to an Amicon® 30K centrifugal filter (VWR) and centrifuged at 4° C. & 4000×g to concentrate.

Relating to adenosine kinetic assays of purified variants: for kinetic parameter assessment of purified ADA2 wt and ADA2v20, a similar protocol was followed from 14. In brief, enzymes were diluted in PBS −/−, pH=7.4. 40 μL of diluted enzyme was pipetted into 96 well plates and then 160 μL of titrated adenosine-PBS solutions were added to analyze A260 decrease over 5 minutes. The initial, linear region taken and adjusted for the in-plate dilution. Standard curves were generated by diluting adenosine solutions in PBS. Michaelis Menten curve fits and resulting k_{cat}/kM parameters were determined with OriginPro 2021.

For lentiviral preparation, 4×10⁶ cells HEK293T cells were seeded in a 10 cm tissue culture (TC) treated dish. The next day, the HEK293T cells were transfected with 5 μg of transfer vector, 1 μg of pMD2.G (Addgene #12259), and 4 μg of psPAX2 (Addgene #12260) with TransIT-LT1 according to the manufacturer's instructions (pMD2.G and psPAX2 were a gift from Dr. Gabe Kwong). After 48 hours, supernatants were spun at 1000×g for 5 minutes to pellet detached cells. Supernatant was then filtered through 0.45 μm PES filters then precipitated with PEG-it™ viral precipitation solution (LV825A-1) according to the manufacturer's instructions. Precipitated virus was resuspended in ice cold PBS −/− (Gibco™) and stored at −80° C. until use. For transduction, 1×10⁶ cells/mL Jurkat cells were spinoculated (2000×g, brakes off) for 90 minutes in media containing virus with 8 μg/mL polybrene (Sigma-Aldrich, #TR-1003). 24 hours after spinoculation, transduced cells were resuspended in fresh media.

For Primary T cell Isolation, Activation, and Transduction: peripheral blood was taken from healthy human donors. PBMCs were isolated using Lymphoprep (STEMCELL, #07801) and SepMate tubes (STEMCELL, #85415) all according to the manufacturer's instructions. Isolated CD3+ T cells were isolated using EasySep Human CD3 Positive Selection Kit (STEMCELL, #17851) and activated with Dynabeads® (ThermoFisher, 11131D) at a 3:1 bead-to-cell ratio. Activated T cells were cultured in Lonza X-Vivo 10 (#04-380Q) supplemented with 5% Human AB Serum (Valley Biomedical, HP1022), 10 mM NAC (Sigma, A9165), 55 μM 2-mercaptoethanol (Sigma, M3148-100ML), and 50 U/mL rhIL-2 (TECIN™ Teceleukin, 23-6019). PEG-it-concentrated lentivirus (MOI=25) was added to untreated 24-well plates coated with retronectin (Takara, T100B) and centrifuged at 1200×g for 90 minutes. Activated T cells were then added to the plate at a cell density of (2×10⁵ cells/mL w/100 U/mL rhIL-2) and spun at 1200×g for 60 minutes and then incubated in the plate for 24 hours. Transduction efficiency was evaluated on day 7 and cells were expanded for 14 days before experimentation

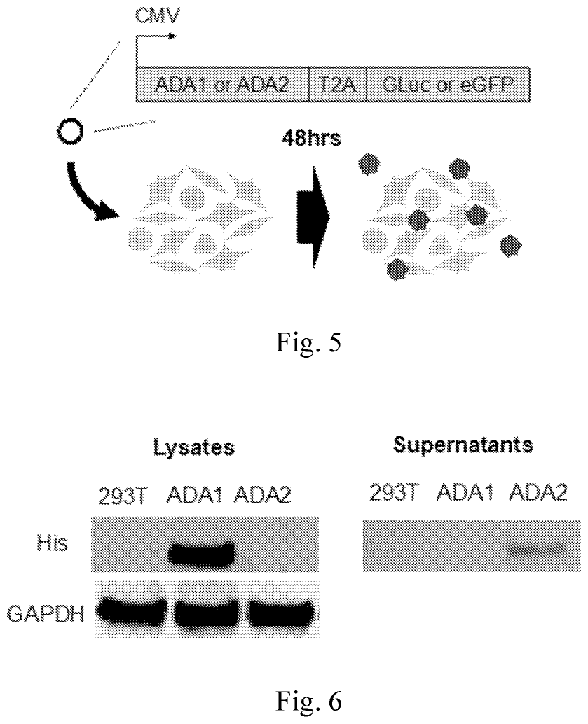

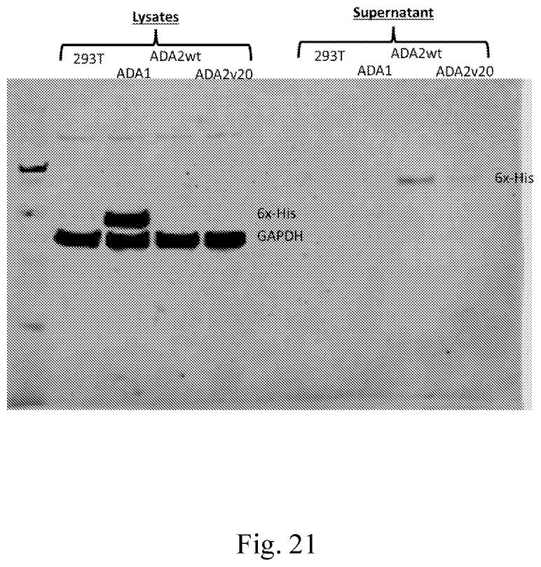

Engineering human cells for extracellular adenosine degradation: to establish whether ADA1 and ADA2 would be superior to degrade extracellular adenosine, the inventors transfected HEK293T cells with pcDNA3 vectors encoding either ADA1 or ADA2 under the control of the CMV promoter. The ADA1/2 coding sequences were followed by a T2A peptide and a Gaussia luciferase (GLuc) or eGFP to allow for normalization based on transfection efficiency (see FIG. 5). Two days after transfection, the inventors added adenosine at a final concentration of 250 μM to cell culture supernatant to assay for extracellular adenosine deaminase activity. Similarly, the inventors tested cell lysate for adenosine deaminase activity to ascertain intracellular ADA activity. While ADA1-mediated extracellular adenosine degradation surpassed that of wildtype ADA2 (˜60 μM/hr versus ˜7 μM/hr in the supernatant), only ˜2% of ADA1 was secreted outside of the cells, compared to ˜100% ADA2 (see FIGS. 6, 27A, 27B). The inventors confirmed these enzyme's tendency towards extracellular secretion or intracellular retention by western blotting of ADA1 and ADA2 expressing HEK293T lysate and supernatant (see FIG. 6, FIG. 21). ADA1 was detectable in cell lysate but not in supernatant, while ADA2 was detectable only in supernatant. The inventors concluded that the higher ADA1 extracellular adenosine deaminase activity compared to ADA2 occurs because ADA1 has a >100× catalytic efficiency (k_{cat}/kM) than ADA2. Thus, even a small percentage (2%) of ADA1 escaping cells is more effective than ADA2 at degrading extracellular adenosine. Experiments were initiated with >95% viability cell cultures to minimize the likelihood of ADA being released from dying cells.

Signal peptides and fusion partners do not improve secretion of the higher activity ADA1 enzyme. As the ADA1 enzyme showed superior extracellular adenosine degradation activity despite being largely intracellularly restricted, the inventors improved its secretion via protein engineering. First, the inventors sought to augment ADA1 secretion by fusing it to secretion signal peptides (SSPs). SSPs are short, ˜15-35 amino acid sequences found in the N-terminus of secreted proteins that are cleaved post-translationally or co-translationally during insertion of the nascent polypeptide into the endoplasmic reticulum, allowing proteins to enter the canonical secretion pathway. It has been shown that intracellular proteins can be engineered to be secreted by fusing them with an efficiently cleaved SSP and the efficacy of protein secretion can be improved by using better signal peptides.

To select SSPs that would enhance ADA1 secretion, the inventors performed an in silico, genome-wide scan for optimal SSP-ADA1 fusions. More specifically, the inventors fused every human SSP taken from a signal peptide database (signalpeptide.de/) to ADA1 protein sequence (without the initiating methionine) in silico, and then predicted the cleavage efficiency and localization profile of each SSP-ADA1 sequence using SignalP 5.0 and DeepLoc. SignalP 5.0 predicts signal peptide cleavage using conditional random field classification and transfer learning, while DeepLoc estimates subcellular localization using recurrent neural networks (see FIG. 11A). The inventors then selected and constructed several SSP-ADA1 fusion proteins with the highest predicted likelihood for extracellular secretion, as well as sequences with varying degrees of predicted cleavage efficiency and extracellular localization (see table 1). The inventors further constructed an experimentally verified modified immunoglobulin kappa (mIgκ) signal peptide-ADA1 fusion protein, as the Igk SSP is routinely used to enhance protein secretion and this modified variant has been shown to allow greater cleavage efficiencies compared to the wildtype sequence.

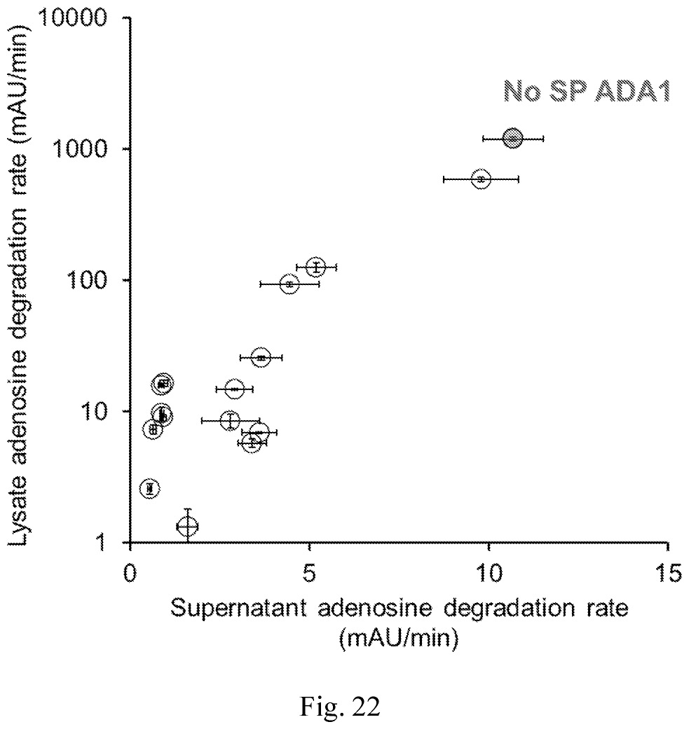

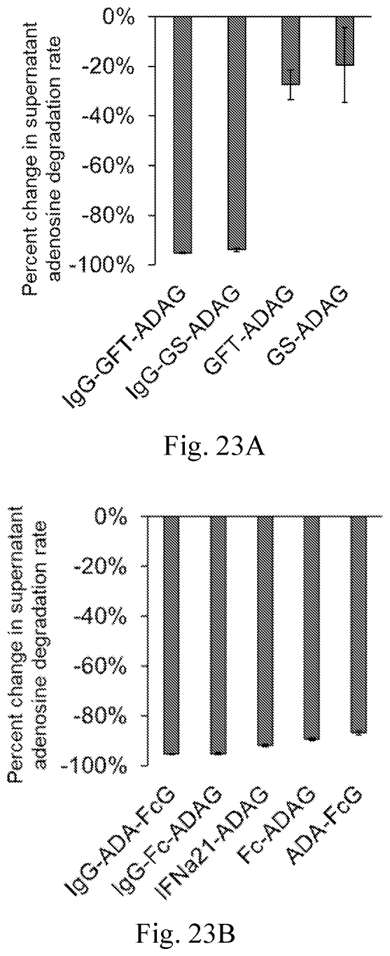

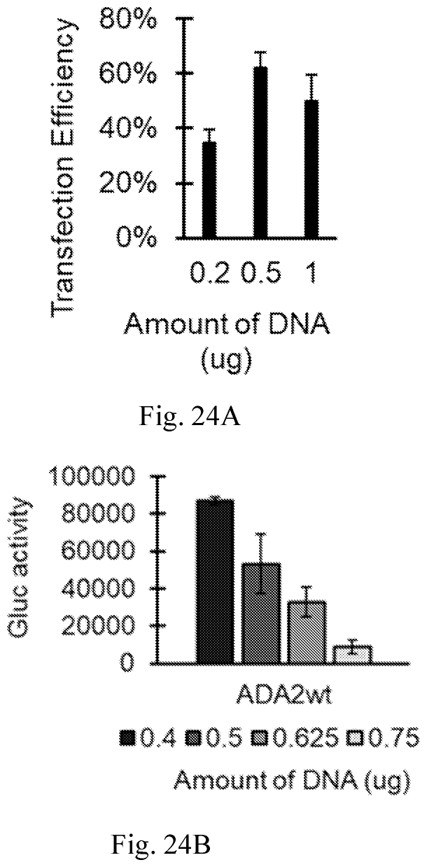

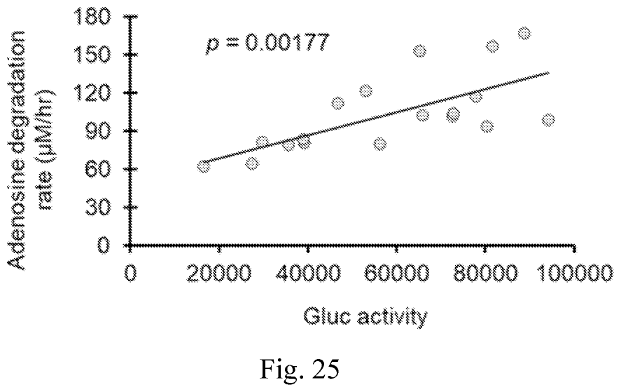

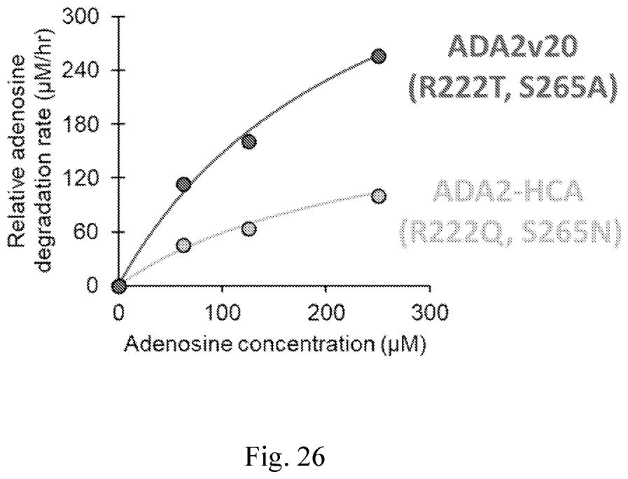

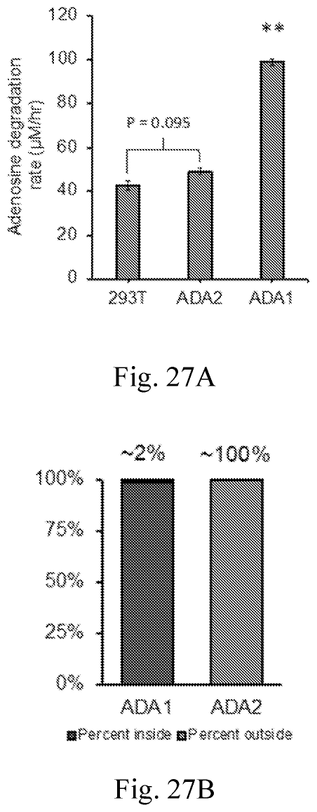

Surprisingly, all SSP-ADA1 combinations reduced the adenosine deaminase activity detected in HEK293T transfected culture supernatant, compared to the unmodified ADA1, despite exhibiting similar transfection efficiencies by eGFP expression. Degradation of adenosine in cell lysate correlated with supernatant degradation rates, suggesting that if the SSP-ADA1 transcripts were translated, they were much more likely to remain intracellular than be secreted (see FIGS. 4, 11B, 22).

Without that, signal peptide cleavage may be failing due to something innate to ADA1's protein structure directly adjacent to the cleavage site. Therefore, the inventors added extra amino acid residues in the form of glycine serine linkers (GS), 3× Flags (F), and Tev protease sites (T) between an IgG signal peptide and ADA1, all of which were unsuccessful in augmenting ADA1 secretion. The inventors also designed constructs with either IgG-Fc domain fusion partners for stability or other small proteins to capitalize on alternative signal peptide cleavage pathways, which were also unsuccessful.

Relating to engineering ADA2 to have enhance its catalytic activity: because ADA1 secretion could not be improved, the inventors sought to engineer the lower-activity ADA2 isoform to have improved kinetic parameters as it is natively secreted into the cell culture supernatant (see FIG. 5). At the entry gate of the active site, ADA2 has many hydrophilic amino acid residues compared to other phylogenetic homologs, which have more hydrophobic gates. These homologs have been shown to have lower kM's, which is sometimes thought of as having improved substrate binding (see table 2). The inventors identified four residues within the active site entry gate of these homologous ADA2s that were more hydrophobic than the Homo sapiens ADA2: E182, R222, S265, H267, which may be responsible for the difference in their respective kMs. Alphafold-predicted structural comparisons of H. sapiens ADA2 and the lowest kM and most hydrophobic homolog, S. peregrina, also show the active site to be considerably more accessible with a larger entry gate. In addition, prior work has demonstrated that a ADA2 variant with R222Q and S265N mutations exhibited a superior kM (˜1 mM) and k_{cat} (169 s⁻¹) and could delay tumor growth in mice.

Guided by their phylogenetic comparison, the inventors constructed a degenerate library of ADA2 variants that would allow for introduction of more hydrophobic residues. Using overlap extension PCR and primer-mediated introduction of nucleotide diversity at positions E182 (E, G, M, V), R222 (R, S, T, Q), S265 (S, A, T, N), and H267 (H, I, V, D), the inventors constructed a library of up to 256 ADA2 variants that was ligated into a pcDNA3 backbone flanked by T2A-Gluc via Golden Gate assembly (see table 3).

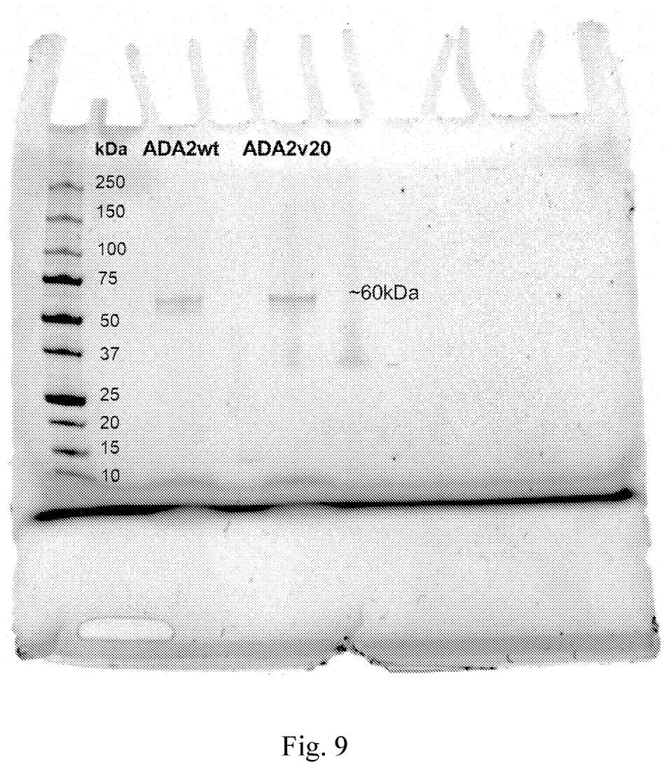

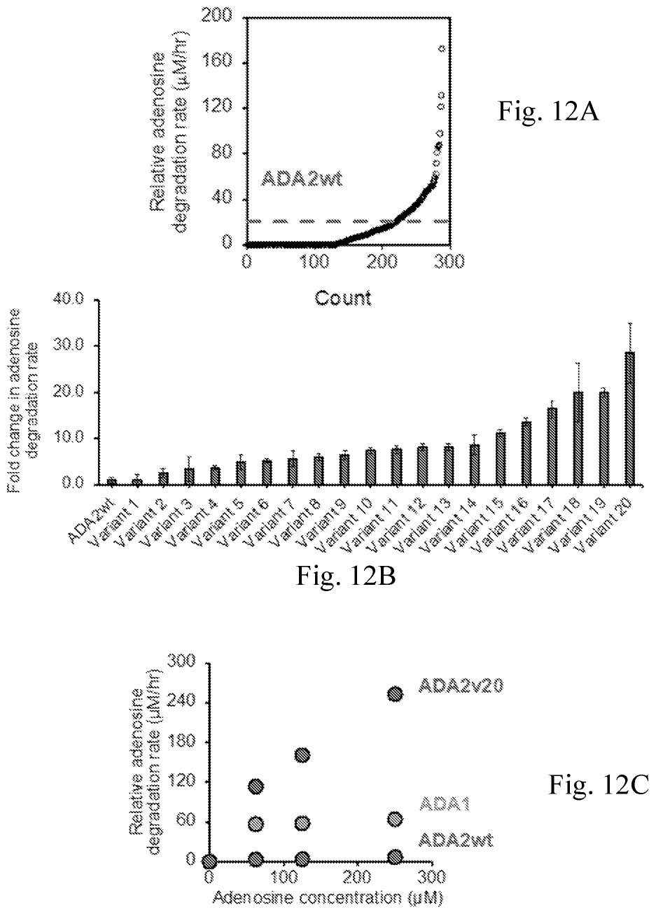

To screen their library at high throughput, the inventors developed a reverse transfection screening technique for HEK293T cells in 96-well plates. 48 hours after transfection, the inventors tested cell culture supernatant for adenosine deaminase activity and GLuc luminescence. The inventors tested ˜300 ADA2 variants, and promisingly, 24% had improved extracellular adenosine deaminase activity than wildtype ADA2 (see FIG. 12A). To account for potential variability during screening, the inventors selected the top 20 variants by ADA activity, rescreened them in sextuplet, and sequenced them to determine their mutational profile (see FIG. 12B). As expected, all 20 variants had at least one mutation, and the top performing ADA2v20 variant (ADA2-R222T/S265A) exhibited an approximate 30-fold increase in adenosine deaminase activity compared to the wildtype enzyme. The inventors isolated this variant twice, as variant 18 also contained the same sequence and a similar fold change adenosine degradation rate (˜20×). Interestingly, this variant also had the same residues at positions 222 and 265 as two homologs from L. longipalpis and S. peregrina (see table 2). The inventors also recovered the previously described ADA2-R222Q/S265N variant from the inventors' library, but saw that it exhibited only a ˜9× increase in adenosine degradation activity at tested conditions (adding 250 μM adenosine to cell culture supernatant). In sum, the inventors successfully engineered the ADA2 binding pocket to be more hydrophobic and accessible, and the highest activity variant exhibited a 20 to 30-fold increase in adenosine degradation rate compared to wildtype enzyme and an approximate 4-fold increase in extracellular degradation compared to ADA1 28. In addition, the ADA2v20 variant retained its native secretion capacity, as confirmed via Western blot. ADA2v20 also shows greatly enhanced extracellular activity compared to ADA2 wt and ADA1 across a broader range of substrate concentrations, (62.5 μM, 125 μM, and 250 μM adenosine) (see FIG. 12C). At 62.5 μM, ADA2v20 outperformed ADA2 wt by ˜35-fold and at 125 μM ADA2v20 outperformed it by ˜50-fold. Notably, ADA2v20 also outperforms the previously reported ADA2-R222Q/S265N variant across this same adenosine concentration range (see FIG. 9).

Relating to purification and kinetic characterization of the engineered ADA2v20 enzyme: to precisely ascertain the kinetic parameters of ADA2v20, the inventors expressed the variant in HEK293T cells through transient transfection, media-changed after 24 hours to serum free media to reduce protein impurities in the supernatant (namely bovine serum albumin), and then purified the ADA2v20 from supernatant using fast pressure liquid chromatography (FPLC). The inventors expressed and purified the ADA2 wt protein for comparison. Both enzymes were >95% pure and exhibit glycosylation heterogeneity, as determined by SDS-PAGE. Using a time-coursed kinetic assay, the inventors determined the Michaelis-Menton kinetic parameters of ADA2 wt (k_{cat}=30 s⁻¹, kM=2.2 mM), which is consistent with previously reported kinetic parameters of ADA2 wt (k_{cat}=45-88 s⁻¹ kM=2.25-4.4 mM), and ADA2v20 (k_{cat}=30 s⁻¹, kM=0.326 mM) (see table 4). Thus, the inventors improved the kM of ADA2 wt approximately 7-fold, while preserving its high k_{cat}.

ADA2 variant 20 is readily secreted by HEK293T, Jurkat, and primary human T cells.

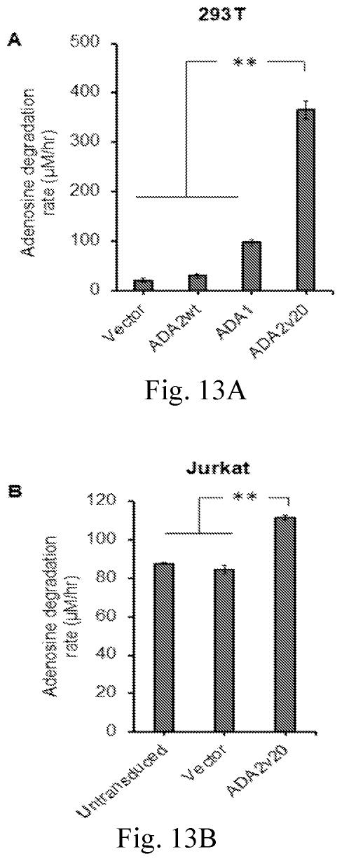

In HEK293T cells, ADA2v20 exhibited a 3.75-fold increase in extracellular adenosine degradation rate compared to ADA1, and a >30× increase in ADA activity compared to ADA2 wt (see FIG. 13A). To ascertain expression levels in an engineered T cell, the inventors first engineered Jurkat T cells with a control tagBFP vector or ADA2v20 to compare their adenosine degradation rates. 48 hours post transduction, the inventors sorted tagBFP+ cells by fluorescence activated cell sorting (FACS). The inventors then seeded 106 cells/mL and sampled their culture supernatant for ADA activity assays. Though Jurkat background is higher due to considerable ADA1 expression, ADA2v20 engineered Jurkat cells still exhibited a 27 μM/hr relative increase in adenosine degradation rate (see FIG. 13B).

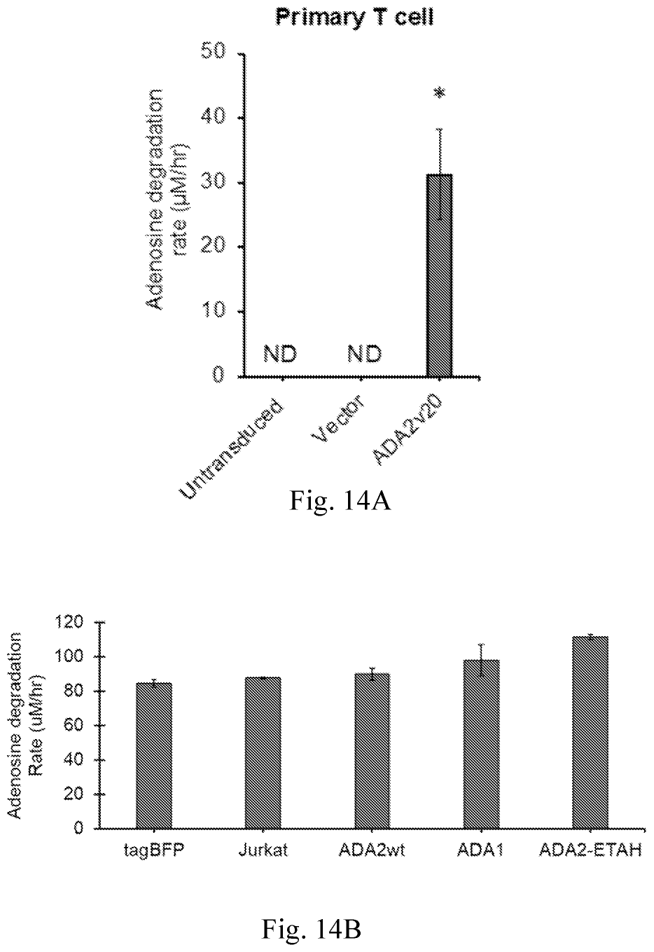

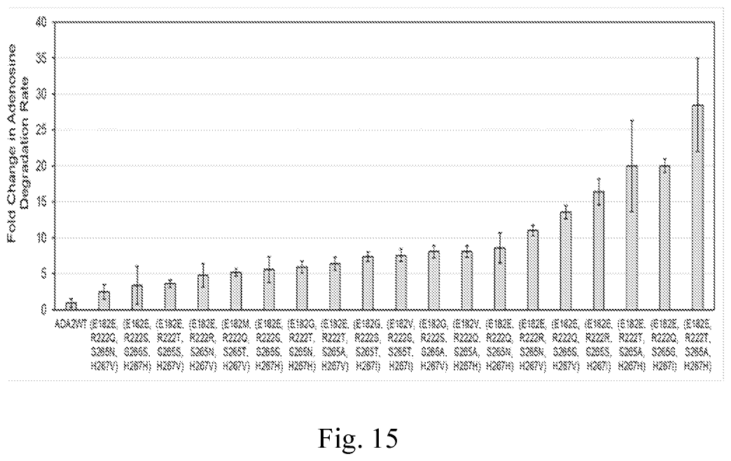

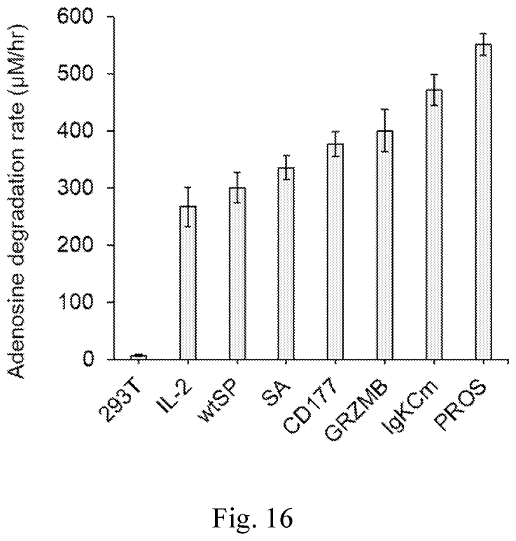

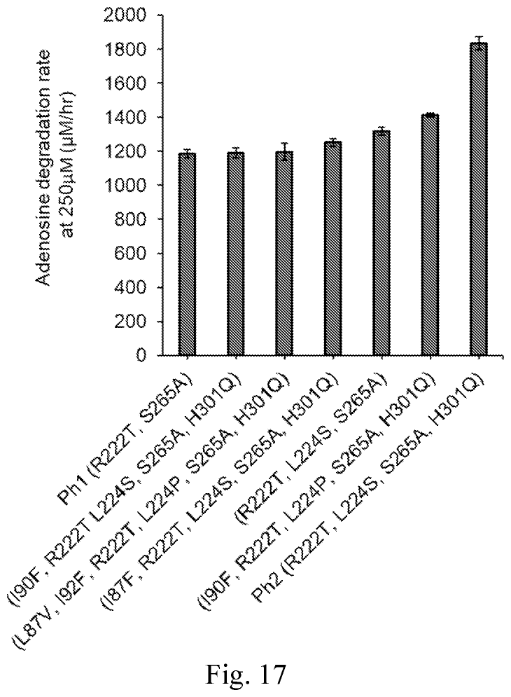

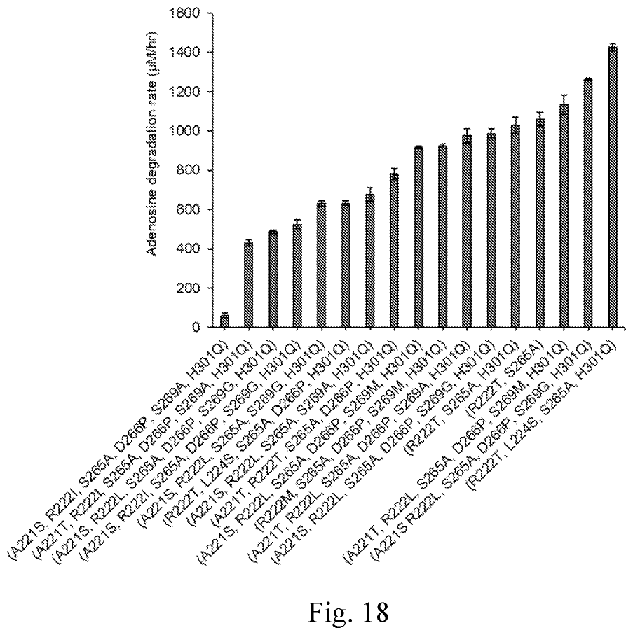

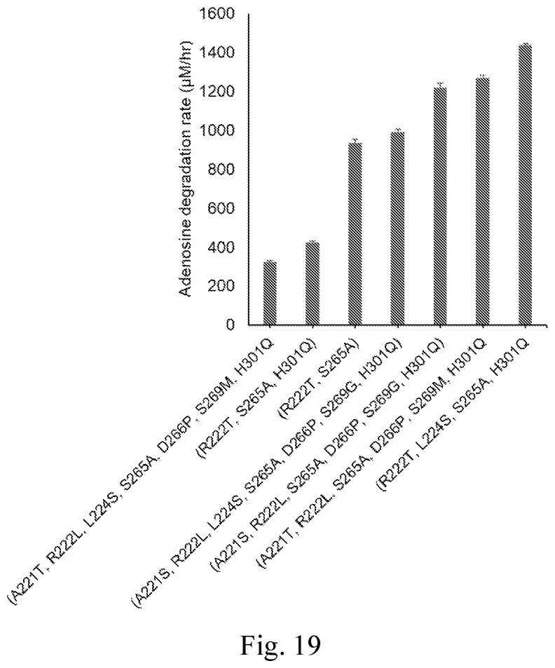

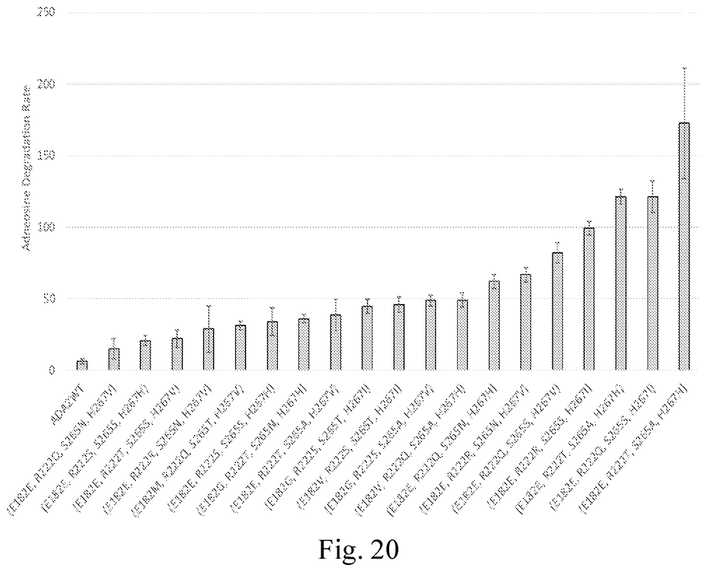

Next, the inventors wanted to test if ADA2v20-engineered primary T cell could degrade a clinically relevant amount of adenosine in vitro. As such, the inventors engineered primary human T cells by transducing them with a vector control or ADA2v20. The transduction efficiency of ADA2v20-engineered T cells was low (˜20%), but the inventors were still able to detect a relative ˜31 μM/hr increase in adenosine degradation, imparted by ADA2v20 (see FIG. 14A).

Enzyme-mediated depletion of tumor accumulated immunosuppressive metabolites can stimulate antitumor immune responses or even an augment to established checkpoint inhibitors (e.g., PD-1 and CTLA-4). The present disclosure demonstrates the feasibility of immunosuppressive metabolite depletion mediated by cellular therapies that have been engineered to secrete metabolite degrading enzymes. The inventors evolved a novel ADA2 enzyme variant for this application that is more considerably more active at tumoral adenosine concentrations than the wildtype enzyme, and demonstrated that human cells secreting this engineered ADA2 variant can degrade tumorally relevant amounts of adenosine.

This treatment modality may provide a path forward to improve the efficacy of CAR T cell therapies, specifically by resisting adenosine-mediated immune suppression. In two separate studies, CRISPR-Cas9-mediated A2AR knockout and ADA1 overexpression were recently shown improve the function of CAR T cell therapies by mitigating T cell susceptibility to adenosine suppression. These two modest improvements can possibly be attributed to fact that adenosine could still signal through the A2BR on A2AR−/− T cells and the extent to which ADA1 overexpressing T cells could secrete ADA1 and degrade adenosine was unclear. Notably, the IL-2 SSP used in the study to secrete ADA1 did not enable functional secretion in the present assays. The present approach here may be more promising as ADA2 is secreted, active, and can thus prevent adenosine from binding any adenosine receptor through systemic depletion into inosine. Moreover, the present approach could theoretically invigorate bystander immune cells as well. So-called ‘armored’ CAR T cell therapies that prevent immune suppression in an autocrine and paracrine manner were recently shown for PD-1-blocking scFv secretion, while also outperforming CAR T cell and αPD-1 coadministration, suggesting that T cell-mediated delivery can provide added benefit by stimulating bystander cells 4. Thus, the inventors engineered both Jurkat and primary T cells with the present evolved ADA2v20 and demonstrated that they could degrade significant amounts of adenosine in vitro extracellularly.

The primary issue limiting the utility of ADA2 for systemic adenosine depletion in tumors (mediated by T cells) was its poor activity at lower concentrations, a property improved by lowering its KM through targeted mutagenesis. ADA1 activity is still superior to ADA2v20, though the variant that the inventors have engineered here is secreted more efficiently and thus considerably more effective at degrading extracellular adenosine in engineered cells than ADA1 and ADA2 wt at micromolar (tumoral) concentrations. Because this R222T/S265A variant is more active at micromolar concentrations of adenosine than the wildtype ADA2 or the previously reported ADA2 variant, it may also be the most suitable ADA2 variant engineered to date for systemic depletion in solid tumor therapeutic applications.

In sum, the present disclosure provides an improved ADA2 variant for adenosine depletion in the solid tumor microenvironment and showed that it is secreted by mammalian cells and significantly more active than the wildtype enzyme at tumoral adenosine concentrations. A question remains whether cellular secretion of ADA1 is possible, but perhaps future approaches could exploit unconventional (i.e., signal peptide independent) mechanisms of secretion. Finally, the inventors performed proof-of-concept studies to demonstrate the feasibility for T cells to secrete this enzyme to be able resist adenosine-mediated immune suppression, setting the stage to be incorporated into a suitable T cell therapy model for preclinical study.

TABLE 1

| SSP | SignalP | DeepLoc |
|---|---|---|
| PROS | 96% | 80% |
| RNAS4 | 84% | 90% |
| CLM9 | 73% | 95% |
| CCl22 | 84% | 82% |
| GRAB | 98% | 67% |
| IgKCm | 90% | 75% |
| HSA | 81% | 78% |
| CATE | 60% | 97% |
| IL19 | 46% | 85% |
| CD177 | 98% | 4% |
| ADA2 | 59% | 2% |
| IL2 | 53% | 0% |
| CYXL | 26% | 0% |
| None | 0 | 0 |

### Example 2

The present example relates to engineered human cells to secrete enzymes that directly degrade adenosine, an immunosuppressive small molecule that has been shown to limit the efficacy of chimeric antigen receptor (CAR) T cell therapies. CAR-T cell therapies are an important class of immunotherapies that is predicted to grow to be a 15B market by 2028 in the US. When secreted by CAR-T and other autologous T cell therapies, the present enzyme weapons grant T cells the ability to resist the detrimental impact of adenosine in solid tumors, improving their efficacy in solid tumors, where CAR-T therapies have typically failed.

In the examples herein, the inventors have engineered human cells to secrete adenosine degrading enzymes. This can be applied to autologous T cells to target ADO in tumors, enhancing their anticancer efficacy. Immune cells have not been engineered to secrete metabolic enzymes that enhance their function against tumors. In addition, the inventors have engineered an enhanced variant of the human adenosine deaminase II (ADA2) enzyme that has 8-fold higher activity than wildtype, as well as several other variants with better kinetic activities compared to wildtype ADA2. Secretion of the strongest enhanced variant allows degradation of a clinically relevant amount of ADO, such that autologous T cells engineered to secrete this variant should be able to resist the impact of ADO in the tumor. This example method of cell-secreted enzyme ‘weaponization’ is broadly applicable therapeutic route to enhance the efficacy of autologous T cells therapies and of chimeric antigen receptor (CAR) T cells in particular. In addition, the engineered ADA2 variant has not been described previously, nor have several other of the ADA2 variants.

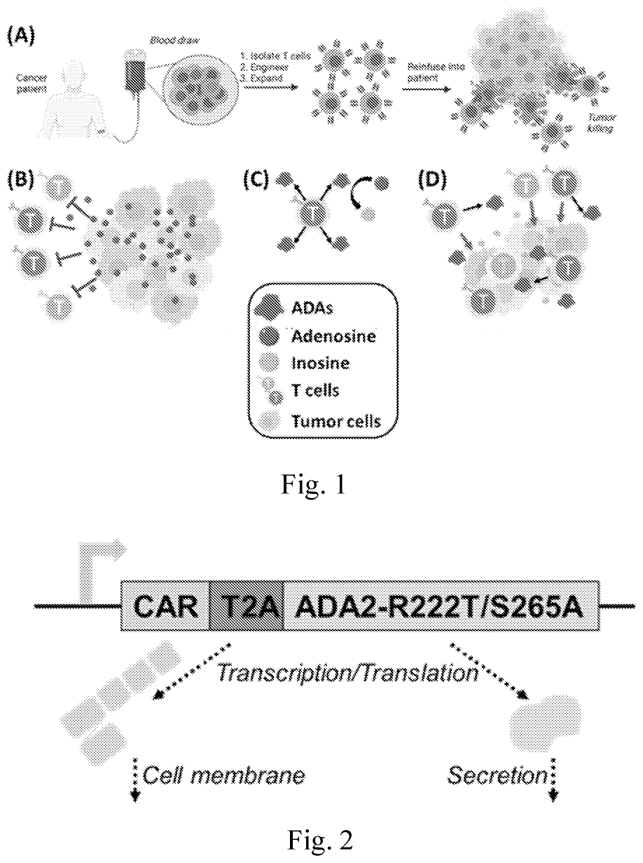

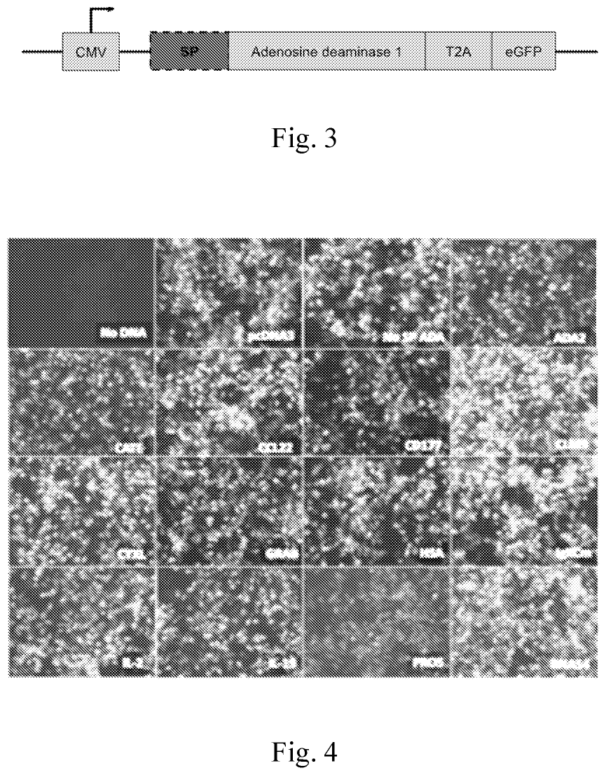

The inventors have shown that it is possible to engineer human cells, including a human T cell line (Jurkat T cells) to secrete metabolic enzymes that degrade adenosine, an immunosuppressive small molecule. Enhancing autologous T cell therapies with the ability to secrete metabolic enzymes should allow them to have enhanced efficacy against solid tumors, as solid tumors accumulate adenosine to shut down T cells (see FIG. 1). In addition, the inventors have engineered a variant of the human adenosine deaminase II (ADA2) enzyme that has 8-fold improved activity. Engineering of Jurkat cells to secrete this ADA2 enzyme variants allows for improved adenosine degrading ability by this T cell line. The inventors have further engineered several other ADA2 enzyme variants with enhanced kinetic activity.

It has been shown that adenosine is an immunosuppressive small molecule that limits the efficacy of engineered and non-engineered autologous T cell therapies (e.g. CAR T cell therapies). The inventors have shown that it is possible to engineer human cells, including Jurkat T cells, to secrete enzymes that degrade adenosine. These examples should be directly applicable to allow creation of T cells therapies with enhanced efficacy against tumors. In addition, the inventors have engineered variants of the ADA2 enzyme with enhanced activity, making them more suitable for secretion by T cell therapies. Recently, it has been shown that engineering T cells to secrete immunomodulatory proteins can enhance their activity against solid tumors, as well as enhancing the activity of bystander T cells. The present examples describe secretion of a new type of immunomodulatory enzyme, a metabolic enzyme that degrades the immune cell inhibiting adenosine molecule. The inventors have shown that Jurkat T cells engineered to secrete the variant of ADA2 with the most activity could degrade a substantial and clinically relevant amount of adenosine.

The DNA sequences of the human adenosine deaminase I (ADA1) and the human adenosine deaminase II (ADA2) enzymes were PCR-amplified from human genomic DNA isolated from HEK293T cells. Both DNA sequences were inserted into the pcDNA human expression vector downstream of the strong pCMV promoter and upstream of a T2A peptide and GLUC (secreted luciferase) gene, generating the following expression cassettes harbored on pCDNA vectors.

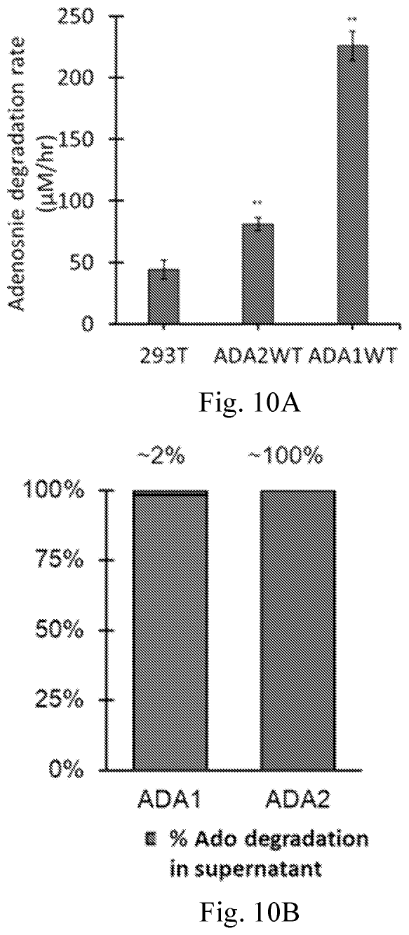

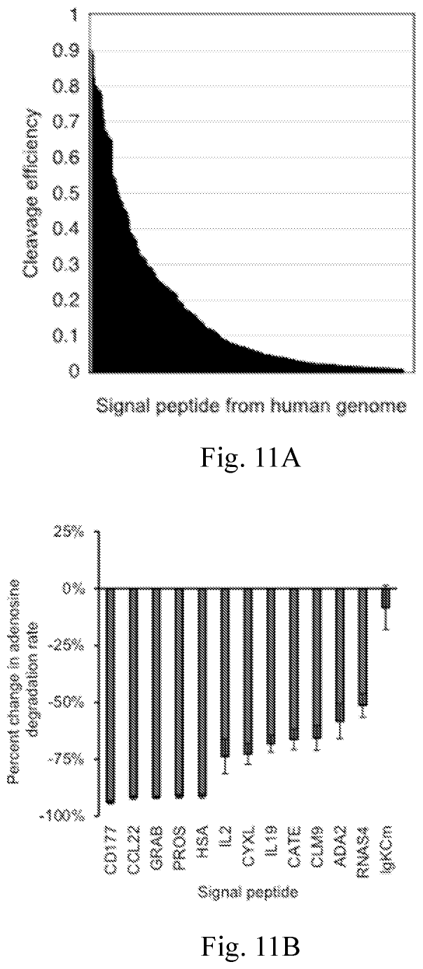

Then, ADA1 and ADA2 encoding pcDNA vectors were transfected into HEK293T cells, which were allowed to recover. To detect human cell secretion of ADA enzymes, 24 hours after cell recovery, the supernatant of each transfected cell type was tested for its ability to degrade adenosine by mixing it with 250 μM of adenosine and then measuring decrease in absorbance at A265 nm. The rate of adenosine degradation for HEK293T cells expressing the ADA1 enzyme or the ADA2 enzyme, as well as control HEK293T cells transfected with an empty pcDNA vector, shows that both ADA1 and ADA2 expression, allow a statistically significant increase in extracellular adenosine degradation compared to control cells (see FIG. 10A). In addition, the lysate of each cell culture was tested for its ability to degrade adenosine and compared to the supernatants ability to degrade adenosine to be able to gauge the relative amounts of extracellular adenosine deaminase activity versus intracellular adenosine deaminase activity. 98% of adenosine deaminase activity for ADA1-expressing HEK293T cells was intracellular, while 100% of adenosine deaminase activity in ADA2-expressing HEK293T cells was extracellular (see FIG. 10B).

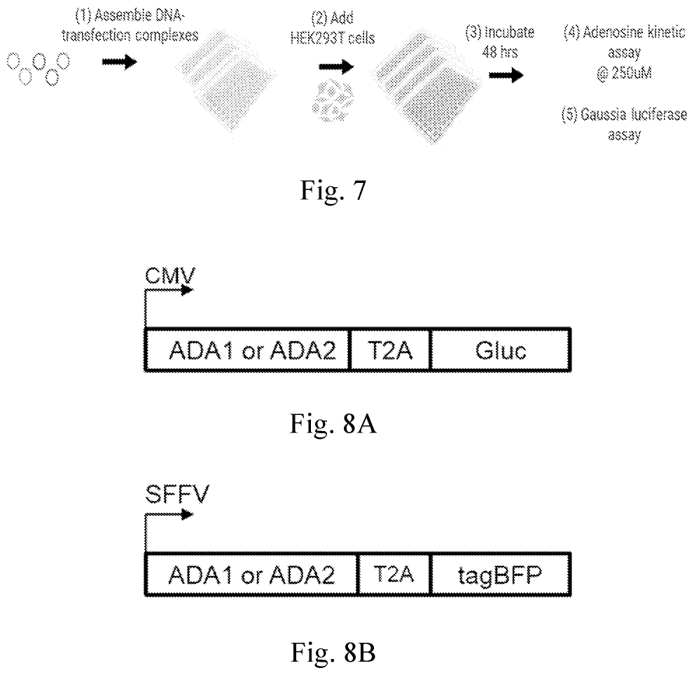

Because adenosine shuts down immune cells and autologous T cell therapies by interacting with extracellular receptors, the inventors focused on enhanced the ability of the ADA2 enzyme, which is secreted extracellularly to degrade ADO (see FIG. 10B). Using overlap extension PCR and molecular cloning techniques, the inventors created a library of ADA2 variants with the following possible mutations E182->E, G, M, V, R222->R, T, S, Q, S265->S, A, T, N, H267->H, I, V, D, encompassing 256 different possible combinations (i.e., different ADA2 mutant variants). The inventors then screened the members of the library for their ability to be secreted from human cells and degrade adenosine with high activity by isolating individual pcDNA plasmids that contained an ADA2 variant being expressed under control of the CMV promoter and flanked by a T2A-Gluc sequence (as shown on the left), and then transfecting individual plasmids into HEK293T cells in a 96-well plate, waiting 48 hours, and then screening the supernatant of each well for adenosine degradation by measured decrease in absorbance at A265 nm that occurred after adding the supernatant to 250 μM adenosine (see FIG. 7). Adenosine degradation rates were normalized by overall expressed, gauged by luciferase levels, and the most promising ADA2 variants were sequences to determine their sequence (see FIG. 5).

Several ADA2 variants allowed for enhanced adenosine degradation in the supernatant of HEK293T cells, with the highest fold increase upon variant expression seen in an ADA2-R222T/S265A showing more than 25-fold improvement in extracellular adenosine degradation.

Relating to the expression and purification of His-tagged HsADA2 Proteins: pcDNA expression vectors harboring either His-tagged wildtype ADA2 (ADA2WT-HisTag) or the His-tagged enhanced activity ADA2-R222T/S265A (ADA2-R222T/S265A-HisTag) variant were transfected into HEK293T cells and recovered and cultured in serum-free media. After 24 hours, the supernatant was harvested, passed through sterile 0.22 μm GD/X Whatman filters (GE Healthcare) and loaded into a 150 mL Superloop (Cytiva). A 5 mL HisTrap High Performance (Cytiva) nickel column was used to purify each enzyme. Buffer A was composed of 20 mM sodium phosphate pH 7.4, 300 mM NaCl, and 20 mM imidazole while Buffer B was composed of 20 mM sodium phosphate pH 7.4, 300 mM NaCl, and 500 mM imidazole. The purification sequence began as a 5 column volume (CV) equilibration of the column with Buffer A, followed by sample application, a 10CV wash with Buffer A, followed by a linear gradient from 0% to 100% Buffer B over 15CV. Fractions were collected in 1 mL aliquots using the Fraction Collector F9-C(Cytiva). All purification steps were performed in a 4° C. refrigeration unit and the flow through the system was 1 mL/minute. Fractions were analyzed by SDS-PAGE, and pure fractions were pooled, sterile filtered using a 0.22 μm filter (Fisher), and concentrated using Amicon Ultra 15 Centrifugal Filter Unit Ultracel-10 (Millipore). Concentrated ADA2WT-HisTag and ADA2-R222T/S265A-HisTagT was buffer exchanged into chilled PBS pH 7.4 using HiTrap Desalting Columns (Cytiva). Protein A280 was determined using the NanoDrop One/OneC UV-Vis Spectrophotometer (Thermo Scientific). Expasy was used to calculate molecular mass and extinction coefficients for His-tagged ADA2 and His-tagged ADA2-R222T/S265A.

For characterization of the activity of ADA2WT and ADA2-R222T/S265A towards adenosine: the inventors evaluated ADA2WT and ADA2-R222T/S265N kinetics with its substrate, adenosine. Michaelis-Menten parameters for each enzyme were determined via a 96-well assay method using substrate concentrations ranging from 0 to 250 μM adenosine and 0.001 μM to 0.005 μM ADA2. In a 96-well UV-Transparent Microplate, 160 μL of 1.25× substrate (in PBS pH 7.4) solution was added to a 40 uL well containing 5× enzyme (in PBS pH 7.4) solution. Absorbance at 265 nm, as a readout of adenosine level, was monitored using a BioTek Synergy HT 96-well plate spectrometer. Adenosine degradation rate was calculated from the linear portion of the raw reaction curves corresponding with less than 10-percent substrate degradation and fitted to the Michaelis-Menten equation using the nonlinear regression solver function Isqnonlin in MATLAB (Mathworks). Non-linear regression analysis revealed catalytic parameters that showed an ˜8-fold increase in activity.
ADA2WT: Km=2228.37 μM; k_{cat}=29.0994 s⁻¹; k_{cat}/kM=13058.6 per M per s
ADA2-R222T/S265A: Km=326.69 μM; k_{cat}=30.30034 s⁻¹; k_{cat}/kM=92750.21 per M per s.

For secretion ADA2WT and ADA2-R222T/S265A from the Jurkat human T cell line: Jurkat T cells were transduced with lentiviral cassettes that contained the following expression construct:

Using this cassette, ADA1, ADA2WT, or ADA2-R222T/S265A were transduced into Jurkat T cells, and then the resulting polyclonal cell mixture was sorted for tagBFP expression to ensure that all Jurkat T cells that were to be tested had received the expression construct. After 24 hours of growth, the level of adenosine deaminase activity was measured in the supernatant of Jurkat T cells harboring transduced lentiviruses (see FIG. 14B). Expression of the ADA2-R222T/S265A variant resulted in a statistically significant increase in adenosine degradation in the cell culture supernatant, at a level of 27 μM adenosine per hour, a clinically relevant amount as this is the expected tumoral accumulation rate of adenosine.

A key advantage of this technology is that no prior method has ever been published/patented that allows human cells, and T cells in particular, to secrete a metabolic enzyme that has an immunomodulatory function. In addition, the HsADA2 enzyme variant to be secreted has enhanced activity and it targets adenosine directly. Therefore, it is not limited by redundancies in adenosine synthesis and signaling, i.e., that multiple metabolic pathways, each of which can employ multiple enzyme homologs, can produce adenosine, and that adenosine suppresses immune cells responses by signaling through multiple receptors found on immune cells). Finally, because this enzyme can be expressed by T cell lines, it could also be secreted by CAR T cells, therapeutically relevant engineered T cells that can specifically target tumor cells (see FIG. 2).

These examples could be used directly as a cancer immunotherapy by engineering autologous T cell therapies like CAR T cells, and high activity adenosine degrading enzymes like ADA2-R222T/S265A can also be used as enzymatic immunotherapies via direct administration of purified enzyme.

A method of introducing the HsADA2 variants into T cells or other immune cells includes lentiviral-mediated transduction. For lentiviral preparation, 4×10⁶ cells HEK293T cells were seeded in a 10 cm tissue culture (TC) treated dish. The next day, the HEK293T cells were transfected with 5 μg of transfer vector, 1 μg of pMD2.G (Addgene #12259), and 4 μg of psPAX2 (Addgene #12260) with TransIT-LT1 according to the manufacturer's instructions (pMD2.G and psPAX2 were a gift from Dr. Gabe Kwong). After 48 hours, supernatants were spun at 1000×g for 5 minutes to pellet detached cells. Supernatant was then filtered through 0.45 μm PES filters then precipitated with PEG-it™ viral precipitation solution (LV825A-1) according to the manufacturer's instructions. Precipitated virus was resuspended in ice cold PBS −/− (Gibco™) and stored at −80° C. until use. For transduction, 1×10⁶ cells/mL Jurkat cells were spinoculated (2000×g, brakes off) for 90 minutes in media containing virus with 8 μg/mL polybrene (Sigma-Aldrich, #TR-1003). 24 hours after spinoculation, transduced cells were resuspended in fresh media.

Peripheral blood was taken from healthy human donors. PBMCs were isolated using Lymphoprep (STEMCELL, #07801) and SepMate tubes (STEMCELL, #85415) all according to the manufacturer's instructions. Isolated CD3+ T cells were isolated using EasySep Human CD3 Positive Selection Kit (STEMCELL, #17851) and activated with Dynabeads® (ThermoFisher, 11131D) at a 3:1 bead-to-cell ratio. Activated T cells were cultured in Lonza X-Vivo 10 (#04-380Q) supplemented with 5% Human AB Serum (Valley Biomedical, HP1022), 10 mM NAC (Sigma, A9165), 55 μM 2-mercaptoethanol (Sigma, M3148-100ML), and 50 U/mL rhIL-2 (TECIN™ Teceleukin, 23-6019). PEG-it-concentrated lentivirus (MOI=25) was added to untreated 24-well plates coated with retronectin (Takara, T100B) and centrifuged at 1200×g for 90 minutes. Activated T cells were then added to the plate at a cell density of (2×10⁵ cells/mL w/100 U/mL rhIL-2) and spun at 1200×g for 60 minutes and then incubated in the plate for 24 hours. Transduction efficiency was evaluated on day 7 and cells were expanded for 14 days before experimentation.

### Example 3—Methods of Making the HsADA2 Variants

A nucleic acid molecule comprising a nucleotide sequence encoding any of the HsADA2 variants as described herein is introduced into a cell via a vector or viral vector, electroporation, or liposome-mediated introduction. The nucleotide sequence encoding the HsADA2 variant(s) is integrated into the host cell genome, the cell is cultured in vitro under conditions suitable for expression and secretion of the HsADA2 variant(s). Preferably, the HsADA2 variant is expressed from a strong promoter (optionally an inducible promoter) and is followed by a terminator.

Suitable host cells include, but are not limited to, mammalian cells such as an immune cell, a cancer cell, a tumor cell, a healthy non-immune cell, a T cell, a CAR T cell, a natural killer (NK) cell, a B cell, and/or a neutrophil.

### Example 4—Methods of Treatment Using the HsADA2 Variants

A therapeutically effective amount of a composition comprising cells comprising a HsADA2 variant and configured to express and secrete the HsADA2 variant, as described herein, is administered to a subject in need thereof, to treat a cancer or tumor in the subject. The cells can be mammalian cells, such as for example and not limitation, an immune cell, a cancer cell, a tumor cell, a healthy non-immune cell, a T cell, a CAR T cell, an NK cell, a B cell, and/or a neutrophil. The composition can further comprise a pharmaceutically acceptable excipient and/or carrier, and can be formulated for any method of administration, such as intratumoral, peritumoral, intradermal, subcutaneous, intravenous, or intraperitoneal administration. The composition can also be administered with one or more additional cancer immunotherapies as discussed herein. It is also contemplated that the cell expressing the HsADA2 variant can be further modified to express and secrete additional immunogenic proteins, or that the cell can be further modified to have inducible control over cell functions.

TABLE 2

| | | Res. | Res. | Res. | Res. |
|---|---|---|---|---|---|
| Species | K_{M} (μM) | 182 | 222 | 265 | 267 |
| H. sapiens | 2228 | E | R | S | H |
| G. gallus | 150 | E | I | A | H |
| X. laevis | 150 | E | M | T | H |
| P. falciparum | 88 | V | S | T | D |
| L. longipalpis | 50 | G | T | A | V |
| S. peregrina | 15 | M | T | A | I |

TABLE 3

| Res. 182 | Res. 222 | Res. 265 | Res. 267 |
|---|---|---|---|
| E | R | S | H |
| G | T | A | I |
| M | S | T | V |
| V | Q | N | D |

TABLE 4

| ADA2 | K_{M} | k_{cat} | k_{cat}/K_{M} |
|---|---|---|---|
| Variant | (μM) | (s⁻¹) | (M⁻¹s⁻¹) |
| H. sapiens | 2228 | 30 | 1.36 × 10⁴ |
| (R222T, S265A) | 326 | 30 | 9.20 × 10⁴ |

TABLE 5

| No. | ADA2 variant | Fold change at 250 μM |
|---|---|---|
| 0 | ADA2WT | 1 |
| 1 | (E182V, R222A, S265N, | 0.92620482 |
| | H267V) | |
| 2 | (R222Q, S265N, H267V) | 2.46536145 |
| 3 | (R222S, S265S) | 3.41716867 |
| 4 | (R222T, S265S, H267V) | 3.64608434 |
| 5 | (R222R, S265N, H267V) | 4.76957831 |
| 6 | (E182M, R222Q, S265T, | 5.15512048 |
| | H267V) | |
| 7 | (R222S) | 5.58885542 |
| 8 | (E182G, R222T, S265N) | 5.91716867 |
| 9 | (R222T, S265A, H267V) | 6.35391566 |
| 10 | (E182G, R222S, S265T, | 7.36596386 |
| | H267I) | |
| 11 | (E182V, R222S, S265T, | 7.56777108 |
| | H2671) | |
| 12 | (E182G, R222S, S265A, | 8.06174699 |
| | H267V) | |
| 13 | (E182V, R222Q, S265A) | 8.07981928 |
| 14 | (R222Q, S265N) | 8.60090361 |
| 15 | (S265N, H267V) | 11.0256024 |
| 16 | (R222Q, H267V) | 13.5406627 |
| 17 | (H267I) | 16.3689759 |
| 18 | (R222T, S265A) | 19.9653614 |
| 19 | (R222Q, H267I) | 19.9864458 |
| 20 | (R222T, S265A) | 28.4623494 |

TABLE 6

| Primer Name | T2A-eGFP |
|---|---|
| jrc 128 | AAATGCCACCATGTGAGACGAGTTCGAGATCGAGGGCGAG (SEQ ID |
| | NO: 103) |
| jrc 129 | GTCCCCGCATGTTAGAAGACTTCCCCTGCCCTCTCCAGAGCCTGAGACG |
| | GCTTCTTGGCCTTGTAGGTG (SEQ ID NO: 104) |
| jrc 130 | GTCTTCTAACATGCGGGGACGTGGAGGAAAATCCCGGCCCAATGGTGA |
| | GCAAGGGCGAGGA (SEQ ID NO: 105) |
| jrc 131 | TGAAGCTGTTACTTGTACAGCTCGTCCATGCC (SEQ ID NO: 106) |
| jrc 132 | CTGTACAAGTAACAGCTTCAGGGATCGTACTTCCA (SEQ ID NO: 107) |
| jrc 133 | GCGTGGGTCCCGCGGTATC (SEQ ID NO: 108) |
| jrc 134 | GATACCGCGGGACCCACGC (SEQ ID NO: 109) |
| jrc 135 | AACTCGTCTCACATGGTGGCATTTCGATAAGCCAGTAAGCAGTGG (SEQ |
| | ID NO: 110) |
| | ADA1-T2A-eGFP/gluc |
| jrc 153 | gatcgatccgtctcacatgGCCCAGACGCCCGCCTTC |
| | (SEQ ID NO: 111) |
| jrc 154 | gatcgatccgtctcaagccGCCTCCATGGTGGTGATGGT (SEQ ID NO: 112) |
| | SP-ADA1-T2A-eGFP |
| jrc 183 | ACTGGCTTATCGAAATGCCACCATGGAGACGCCCCGCCGACATCCCCG |
| | ACTACTTGCGTCTCGCCCAGACGCCCGCCTTC (SEQ ID NO: 113) |
| jrc 184 | ACTTCCCCTGCCCTCTCCAG (SEQ ID NO: 114) |
| | Signal peptides |
| ADA2 5′ | CCATGTTGGTGGATGGCCCATCTGAGCGGCCAGCCCTGTGCTTCTTGCT |
| | GTTGGCTGTGGCAATGTCTTTCTTCGGCTCAGCTCTATCCG (SEQ ID NO: |
| | 115) |
| ADA2 3′ | TGGGCGGATAGAGCTGAGCCGAAGAAAGACATTGCCACAGCCAACAG |
| | CAAGAAGCACAGGGCTGGCCGCTCAGATGGGCCATCCACCAAC (SEQ |
| | ID NO: 116) |
| HSA5′ | CCATGAAGTGGGTAACCTTTATTTCCCTTCTTTTTCTCTTTAGCTCGGCT |
| | TATTCCG (SEQ ID NO: 117) |
| HSA3′ | TGGGCGGAATAAGCCGAGCTAAAGAGAAAAAGAAGGGAAATAAAGGT |
| | TACCCACTTC (SEQ ID NO: 118) |
| IgKCm 5′ | CCATGGGCAGCGCCGCTCTCCTGCTCTGGGTGCTGCTGCTGTGGGTGCC |
| | CAGCTCCAGGGCCG (SEQ ID NO: 119) |
| IgKCm 3′ | TGGGCGGCCCTGGAGCTGGGCACCCACAGCAGCAGCACCCAGAGCAG |
| | GAGAGCGGCGCTGCCC (SEQ ID NO: 120) |
| IL-2 5′ | CCATGTACAGGATGCAACTCCTGTCTTGCATTGCACTAAGTCTTGCACT |
| | TGTCACAAACAGTG (SEQ ID NO: 121) |
| IL-2 3′ | TGGGCACTGTTTGTGACAAGTGCAAGACTTAGTGCAATGCAAGACAGG |
| | AGTTGCATCCTGTAC (SEQ ID NO: 122) |
| CYXL 5′ | CCATGGCCCGCGCAGCCCCGCTGCTCGCCGCGTTGACCGCGCTCCTCGC |
| | CGCCGCCGCTGCTGGCGGAG (SEQ ID NO: 123) |
| CYXL 3′ | TGGGCTCCGCCAGCAGCGGCGGCGGCGAGGAGCGCGGTCAACGCGGC |
| | GAGCAGCGGGGCTGCGCGGGCC (SEQ ID NO: 124) |
| IL19 5′ | CCATGAAGTTACAGTGTGTTTCCCTTTGGCTCCTGGGTACAATACTGAT |
| | ATTGTGCTCAGTAGACAACCACGGTG (SEQ ID NO: 125) |
| IL19 3′ | TGGGCACCGTGGTTGTCTACTGAGCACAATATCAGTATTGTACCCAGG |
| | AGCCAAAGGGAAACACACTGTAACTTC (SEQ ID NO: 126) |
| CD177 5′ | CCATGAGCGCGGTATTACTGCTGGCCCTCCTGGGGTTCATCCTCCCACT |
| | GCCAGGAGTGCAGGCGG (SEQ ID NO: 127) |
| CD177 3′ | TGGGCCGCCTGCACTCCTGGCAGTGGGAGGATGAACCCCAGGAGGGCC |
| | AGCAGTAATACCGCGCTC (SEQ ID NO: 128) |
| PROS 5′ | CCATGAGGGTCCTGGGTGGGCGCTGCGGGGCGCTGCTGGCGTGTCTCC |
| | TCCTAGTGCTTCCCGTGTCAGAGGCAG (SEQ ID NO: 129) |
| PROS 3′ | TGGGCTGCCTCTGACACGGGAAGCACTAGGAGGAGACACGCCAGCAG |
| | CGCCCCGCAGCGCCCACCCAGGACCCTC (SEQ ID NO: 130) |
| RNAS4 5′ | CCATGGCTCTGCAGAGGACCCATTCATTGCTTCTGCTTTTGCTGCTGAC |
| | CCTGCTGGGGCTGGGGCTGGTCCAGCCCTCCTATGGCG (SEQ ID NO: |
| | 131) |
| RNAS4 3′ | TGGGCGCCATAGGAGGGCTGGACCAGCCCCAGCCCCAGCAGGGTCAG |
| | CAGCAAAAGCAGAAGCAATGAATGGGTCCTCTGCAGAGCC (SEQ ID |
| | NO: 132) |
| CCL22 5′ | CCATGGATCGCCTACAGACTGCACTCCTGGTTGTCCTCGTCCTCCTTGC |
| | TGTGGCGCTTCAAGCAACTGAGGCAG (SEQ ID NO: 133) |
| CCL22 3′ | TGGGCTGCCTCAGTTGCTTGAAGCGCCACAGCAAGGAGGACGAGGACA |
| | ACCAGGAGTGCAGTCTGTAGGCGATCC (SEQ ID NO: 134) |
| GRAB 5′ | CCATGCAACCAATCCTGCTTCTGCTGGCCTTCCTCCTGCTGCCCAGGGC |
| | AGATGCAG (SEQ ID NO: 135) |
| GRAB 3′ | TGGGCTGCATCTGCCCTGGGCAGCAGGAGGAAGGCCAGCAGAAGCAG |
| | GATTGGTTGC (SEQ ID NO: 136) |
| CATE 5′ | CCATGAAAACGCTCCTTCTTTTGCTGCTGGTGCTCCTGGAGCTGGGAGA |
| | GGCCCAAGGAG (SEQ ID NO: 137) |
| CATE 3′ | TGGGCTCCTTGGGCCTCTCCCAGCTCCAGGAGCACCAGCAGCAAAAGA |
| | AGGAGCGTTTTC (SEQ ID NO: 138) |
| CLM9 5′ | CCATGCGGCTTCTGGTCCTGCTATGGGGTTGCCTGCTGCTCCCAGGTTA |
| | TGAAGCCG (SEQ ID NO: 139) |
| CLM9 3′ | TGGGCGGCTTCATAACCTGGGAGCAGCAGGCAACCCCATAGCAGGACC |
| | AGAAGCCGC (SEQ ID NO: 140) |
| | ADA2-T2A-gluc/eGFP Constructs |
| jrc 190 | gatcgatccgtctcacatgACGTCGTCCGTCTCACATGT (SEQ ID NO: 141) |
| jrc 191 | gatcgatccgtctcaagccAGCATGCACGTCTCAAGCC |
| | (SEQ ID NO: 142) |
| | Fusion Constructs |
| jrc202 | AGCTGACAAGCGTCTCACCATGGCCCTGTCCTTTTCTTTACTGAT (SEQ |
| | ID NO: 143) |
| jrc203 | TTCATCGACTCGTCTCCTGGGCGCTCCCGCCACCTCCACTCCCGCCGCC |
| | ACCTTCCTTCCTCCTTAATCTTTCTTGAAAA (SEQ ID NO: 144) |
| jrc204 | AGCTGACAAGCGTCTCAGGAGGTGGAGGAAGCGG (SEQ ID NO: 145) |
| jrc205 | TTCATCGACTCGTCTCCTGGGCGCCCTGGAAGTAGAGGTTTTC (SEQ ID |
| | NO: 146) |
| jrc206 | AGCTGACAAGCGTCTCAGGTGGTGGTGGTAGCGGT (SEQ ID NO: 147) |
| jrc207 | TTCATCGACTCGTCTCCTGGGCACTCCCGCCCCCACCG (SEQ ID NO: |
| | 148) |
| IgG1 5′ | CCATGCGGCCTACTTGGGCCTGGTGGCTGTTCCTGGTGCTGCTGCTAGC |
| Linkers | TCTGTGGGCACCCGCTAGAGGG (SEQ ID NO: 149) |
| IgG1 3′ | GCCACCAGGCCCAAGTAGGCCGC (SEQ ID NO: 150) |
| Linkers GFT | CTCCCCCTCTAGCGGGTGCCCACAGAGCTAGCAGCAGCACCAGGAACA |
| IgG1 3′ | CACCCCCTCTAGCGGGTGCCCACAGAGCTAGCAGCAGCACCAGGAACA |
| Linkers GS | GCCACCAGGCCCAAGTAGGCCGC (SEQ ID NO: 151) |
| jrc208 | AGCTAGCAAGCGTCTCACCTCGGAGGAGGTGGCTCTGGC (SEQ ID NO: |
| | 152) |
| jrc209 | AGCTAGCAAGCGTCTCAAGCCGCCTCCATGGTGGTGATGGT (SEQ ID |
| | NO: 153) |
| IgG1 5′ | CATGCGGCCTACTTGGGCCTGGTGGCTGTTCCTGGTGCTGCTGCTAGCT |
| ADAFc | CTGTGGGCACCCGCTAGAGGG (SEQ ID NO: 154) |
| IgG1 3′ | GGGCCCCTCTAGCGGGTGCCCACAGAGCTAGCAGCAGCACCAGGAAC |
| ADAFc | AGCCACCAGGCCCAAGTAGGCCG (SEQ ID NO: 155) |
| jrc210 | AGCTAGCAAGCGTCTCAGCCCAGACGCCCGCCTT (SEQ ID NO: 156) |
| jrc211 | AGCTAGCAAGCGTCTCAGAGGTTCTGCCCTGCAGAGG (SEQ ID NO: 157) |
| jrc212 | AGCTAGCAAGCGTCTCACATGGCCCAGACGCCCGCCTT (SEQ ID NO: 158) |
| jrc213 | AGCTAGCAAGCGTCTCAACTCACACATCCCCACCGA (SEQ ID NO: 159) |
| jrc214 | AGCTAGCAAGCGTCTCATTTACCCGGAGACAGGGAGA (SEQ ID NO: |
| | 160) |
| jrc215 | AGCTAGCAAGCGTCTCATAAAGGAGGAGGTGGCTCTGGCGGTGGAGGT |
| | TCCGCCCAGACGCCCGCCTTC (SEQ ID NO: 161) |
| jrc216 | AGCTAGCAAGCGTCTCAAGCCGCCTCCATGGTGGTGATGGTG (SEQ ID |
| | NO: 162) |
| IgG 3′ | GAGTCCCTCTAGCGGGTGCCCACAGAGCTAGCAGCAGCACCAGGAACA |
| FcADA | GCCACCAGGCCCAAGTAGGCCG (SEQ ID NO: 163) |
| jrc217 | AGCTAGCAAGCGTCTCACATGACTCACACATCCCCACCG (SEQ ID NO: |
| | 164) |
| IgG1 3′ SP | TGGGCCCCTCTAGCGGGTGCCCACAGAGCTAGCAGCAGCACCAGGAAC |
| | AGCCACCAGGCCCAAGTAGGCCGC (SEQ ID NO: 165) |
| jrc218 | AGCTGACAAGCGTCTCACCATGGGAGGTGGAGGAAGCGG (SEQ ID NO: |
| | 166) |
| jrc219 | AGCTGACAAGCGTCTCACCATGGGTGGTGGTGGTAGCGGT (SEQ ID NO: |
| | 167) |
| jrc228 | GCTACTGACGTCTCACATGGCCCAGACGCCCGCCTT (SEQ ID NO: 168) |
| jrc229 | GCTACTGACGTCTCAAGCCGCCTCCATGGTGGTGATGGT (SEQ ID NO: |
| | 169) |
| jrc230 | GCTACTGACGTCTCACATGACTCACACATCCCCACCGAG (SEQ ID NO: |
| | 170) |

TABLE 7

| | Making ADA2-HCA (R222Q, S265N) |
|---|---|
| jrc222 | GCTTACTGGCTTATCGAAATGCCACCATGTTGGTGGATGGCCCATC |
| | (SEQ ID NO: 171) |
| jrc223 | CCGGCAGCAGCTGGGCTCTGATC (SEQ ID NO: 172) |
| jrc224 | GATCAGAGCCCAGCTGCTGCCGG (SEQ ID NO: 173) |
| jrc225 | TTTGGATCTGTGATCGTTATAAATGATTTTGAT (SEQ ID NO: 174) |
| jrc226 | ATCAAAATCATTTATAACGATCACAGATCCAAA (SEQ ID NO: 175) |
| jrc227 | AGAAGACTTCCCCTGCCCTCTCCAGAGCCGCCTCCATGGTGGTGATG |
| | (SEQ ID NO: 176) |
| | ADAs into Lentivirus |
| jrc242 | TCCGATTGACTGAGTCGCCCGGATCCCGCCACCATGTTGGTGGATGGC |
| | CCATC (SEQ ID NO: 177) |
| jrc243 | AAGTTATTAGGTCCCTCGACGAATTCTTACTTGTACAGCTCGTCCATG |
| | (SEQ ID NO: 178) |
| jrc244 | TGGTGAAGAAGATGGTTGTAAATTTCGACCAGACA (SEQ ID NO: 179) |
| jrc245 | TGTCTGGTCGAAATTTACAACCATCTTCTTCACCA(SEQ ID NO: 180) |
| | Library |
| jrc278 | GTCTTCTAACATGCGGGGACGTGGAGGAAAATCCCGGCCCAATGGGA |
| | GTCAAAGTTCTGTTTGCCCT (SEQ ID NO: 181) |
| jrc279 | GTACGATCCCTGAAGCTGTTAGTCACCACCGGCCCCCTT (SEQ ID NO: |
| | 182) |
| jrc280 | CAGCTTCAGGGATCGTACTTCCAA (SEQ ID NO: 183) |
| jrc287 | cgtactgacgtctcacATGGTGAGCAAGGGCGAGG (SEQ ID NO: 184) |
| jrc288 | gatccatgcgtctcaagccCTTGTACAGCTCGTCCATGCC (SEQ ID NO: 185) |
| jrc289 | ACCATCACCACCATGGAGGCGGCTCTGGAGAGGGCAGG (SEQ ID NO: |
| | 186) |
| E182(EGV)f | ATGTTGTCTGGTCGAAATTTGDAACCATCTTCTTCACCATCTC (SEQ ID |
| | NO: 187) |
| E182(M)f | ATGTTGTCTGGTCGAAATTTATGACCATCTTCTTCACCATCTC (SEQ ID |
| | NO: 188) |
| R222(RQ)f | TCTACATGGAGATCAGAGCCCRACTGCTGCCGGTGTATGAGCT (SEQ |
| | ID NO: 189) |
| R222(ST)f | TCTACATGGAGATCAGAGCCASCCTGCTGCCGGTGTATGAGCT (SEQ |
| | ID NO: 190) |
| S265(AT) | CCCTGAGTTTATTGGAATCAAAATCATTTATRCAGATSACAGATCCAA |
| H267(HD)f | AGATGTGGCTGTCATC (SEQ ID NO: 191) |
| S265(AT) | CCCTGAGTTTATTGGAATCAAAATCATTTATRCAGATRTAAGATCCAA |
| H267(IV)f | AGATGTGGCTGTCATC (SEQ ID NO: 192) |
| S265(SN) | CCCTGAGTTTATTGGAATCAAAATCATTTATARCGATRTAAGATCCAA |
| H267(HD)f | AGATGTGGCTGTCATC (SEQ ID NO: 193) |
| S265(SN) | CCCTGAGTTTATTGGAATCAAAATCATTTATARCGATSACAGATCCAA |
| H267(IV)f | AGATGTGGCTGTCATC (SEQ ID NO: 194) |
| 181rev | AAATTTCGACCAGACAACAT (SEQ ID NO: 195) |
| 221rev | GGCTCTGATCTCCATGTAGA (SEQ ID NO: 196) |
| 264rev | ATAAATGATTTTGATTCCAATAAACTCAGGG (SEQ ID NO: 197) |
| farCMVfwd | GTTTGACTCACGGGGATTTC (SEQ ID NO: 198) |
| farGFPrev | CCGGTGGTGCAGATGAACTT (SEQ ID NO: 199) |
| ggADA2fwd | gatccatgCGTCTCacatgTTGGTGGATGGCCCAT (SEQ ID NO: 200) |
| ggADA2rev | catgacgtCGTCTCaagccGCCTCCATGGTGGTGATGGT (SEQ ID NO: 201) |
| | Protein Expression/Purification Constructs |
| jrc298 | GCTGGCTAGCGTTTAAACTTAAGCTTGCCACCATGTTGGTGGATG |
| | (SEQ ID NO: 202) |
| jrc299 | CAGCGGGTTTAAACGGGCCCTCTAGACTAGCCTCCATGGTGGTGA |
| | (SEQ ID NO: 203) |

>HsADA2 with C terminal 6xHis tag with sequence GGHHHHHHGG
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCAGGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATTCGGATCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRARLLPVYELSGEHHDEEWSVKTYQEVAQK
FVETHPEFIGIKIIYSDHRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(R222Q, S265N, H267V) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCCAGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATAATGATGTGA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRAQLLPVYELSGEHHDEEWSVKTYQEVAQK
FVETHPEFIGIKIIYNDVRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(R222S) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCTCGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATTCGGATCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRASLLPVYELSGEHHDEEWSVKTYQEVAQK
FVETHPEFIGIKIIYSDHRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(R222T, H267V) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCACCCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATTCGGATGTGA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRATLLPVYELSGEHHDEEWSVKTYQEVAQK
FVETHPEFIGIKIIYSDVRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(S265N, H267V) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCAGGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATAACGATGTGA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRARLLPVYELSGEHHDEEWSVKTYQEVAQK
FVETHPEFIGIKIIYNDVRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(E182M, R222Q, S265T, H267V) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTATGACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCCAGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATACGGATGTGA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCELLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFMTIFFTISGLIHY
APVFRDYVFRSMQEFYEDNVLYMEIRAQLLPVYELSGEHHDEEWSVKTYQEVAQK
FVETHPEFIGIKIIYTDVRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(E182G, R222T, S265N) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGGCACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCACCCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATAACGATCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFGTIFFTISGLIHY
APVFRDYVFRSMQEFYEDNVLYMEIRATLLPVYELSGEHHDEEWSVKTYQEVAQK
FVETHPEFIGIKIIYNDHRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(R222T, S265A, H267V) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCACCCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATAACGATGTGA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRATLLPVYELSGEHHDEEWSVKTYQEVAQK
FVETHPEFIGIKIIYADVRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(E182G, R222S, S265T, H267I) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGGCACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCTCACTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATACCGATATAA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFGTIFFTISGLIHY
APVFRDYVFRSMQEFYEDNVLYMEIRASLLPVYELSGEHHDEEWSVKTYQEVAQK
FVETHPEFIGIKIIYTDIRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(E182V, R222S, S265T, H267I) with 6xHis tag
(Seq ID No. .)
ATGTTGGTGGATGGCCCATCTGAGCGGCCAGCCCTGTGCTTCTTGC
TGTTGGCTGTGGCAATGTCTTTCTTCGGCTCAGCTCTATCCATAGATGAAACACG
GGCGCATCTGTTGTTGAAAGAAAAGATGATGCGGCTGGGGGGGCGGCTGGTGCTG
AACACCAAGGAGGAGCTGGCCAATGAGAGGCTCATGACGCTCAAAATCGCTGAGA
TGAAGGAGGCCATGAGGACCCTGATATTCCCACCCAGCATGCACTTTTTCCAGGC
CAAGCATCTCATTGAGAGAAGTCAAGTGTTTAATATTCTAAGGATGATGCCAAAA
GGGGCTGCCTTGCACCTCCATGACATTGGCATCGTGACTATGGACTGGCTGGTGA
GGAATGTCACCTACAGGCCTCACTGCCACATCTGTTTCACCCCAAGGGGGATCAT
GCAGTTCAGATTTGCTCACCCAACTCCCCGTCCATCAGAAAAATGTTCCAAGTGG
ATTCTGCTGGAGGATTATCGGAAGCGGGTGCAGAACGTCACTGAGTTTGATGACA
GCTTGCTGAGGAATTTCACTCTGGTGACCCAGCACCCGGAGGTGATTTACACAAA
CCAAAATGTTGTCTGGTCGAAATTTGTAACCATCTTCTTCACCATCTCTGGTCTC
ATCCATTACGCACCAGTGTTCAGAGACTATGTCTTCCGGAGCATGCAGGAGTTCT
ACGAGGACAACGTGCTCTACATGGAGATCAGAGCCTCACTGCTGCCGGTGTATGA
GCTCAGTGGAGAGCACCATGACGAAGAGTGGTCAGTGAAGACTTACCAGGAAGTA
GCTCAGAAGTTTGTGGAAACTCACCCTGAGTTTATTGGAATCAAAATCATTTATA
CCGATATAAGATCCAAAGATGTGGCTGTCATCGCAGAATCCATCCGAATGGCCAT
GGGGCTCCGAATCAAGTTCCCCACGGTGGTGGCAGGGTTTGACCTGGTGGGGCAT
GAGGACACTGGCCACTCCTTGCATGACTACAAGGAAGCTCTGATGATCCCCGCCA
AGGATGGCGTTAAGCTGCCTTACTTCTTCCACGCCGGAGAAACAGACTGGCAGGG
TACTTCCATAGACAGGAACATTCTGGATGCTCTGATGCTGAACACTACCAGAATC
GGCCATGGATTTGCTTTGAGCAAACACCCCGCAGTCAGGACTTACTCCTGGAAAA
AGGACATCCCCATAGAAGTCTGTCCCATCTCTAACCAGGTGCTGAAACTGGTGTC
TGACTTGAGGAACCACCCTGTAGCCACTCTGATGGCCACTGGGCACCCCATGGTG
ATCAGCTCTGATGACCCAGCTATGTTTGGTGCCAAAGGCTTGTCCTATGATTTCT
ATGAGGTCTTCATGGGCATTGGGGGGATGAAGGCTGACCTGAGGACCCTCAAACA
GCTGGCCATGAACTCTATCAAGTACAGTACCCTGTTGGAGAGTGAGAAAAATACT
TTCATGGAAATCTGGAAGAAGAGATGGGATAAGTTCATAGCAGATGTGGCTACAA
AGGGCGGACACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFVTIFFTISGLIHY
APVFRDYVFRSMQEFYEDNVLYMEIRASLLPVYELSGEHHDEEWSVKTYQEVAQK
FVETHPEFIGIKIIYTDIRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(E182G, R222S, S265A, H267V) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGGCACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCTCACTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCAGATGTGA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFGTIFFTISGLIHY
APVFRDYVFRSMQEFYEDNVLYMEIRASLLPVYELSGEHHDEEWSVKTYQEVAQK
FVETHPEFIGIKIIYADVRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(E182V, R222Q, S265A) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGTGACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCCAGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCAGATCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFVTIFFTISGLIHY
APVFRDYVFRSMQEFYEDNVLYMEIRAQLLPVYELSGEHHDEEWSVKTYQEVAQK
FVETHPEFIGIKIIYADHRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(S265N, H267V) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCAGGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATAACGATGTGA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRARLLPVYELSGEHHDEEWSVKTYQEVAQK
FVETHPEFIGIKIIYNDVRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(R222Q, H267V) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCCAGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATTCGGATGTGA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRAQLLPVYELSGEHHDEEWSVKTYQEVAQK
FVETHPEFIGIKIIYSDVRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(H2671) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCAGGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATTCGGATATAA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRARLLPVYELSGEHHDEEWSVKTYQEVAQK
FVETHPEFIGIKIIYSDIRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(R222Q, H2671) with 6xHis tag
(Seq ID No. .)
ATGTTGGTGGATGGCCCATCTGAGCGGCCAGCCCTGTGCTTCTTGC
TGTTGGCTGTGGCAATGTCTTTCTTCGGCTCAGCTCTATCCATAGATGAAACACG
GGCGCATCTGTTGTTGAAAGAAAAGATGATGCGGCTGGGGGGGCGGCTGGTGCTG
AACACCAAGGAGGAGCTGGCCAATGAGAGGCTCATGACGCTCAAAATCGCTGAGA
TGAAGGAGGCCATGAGGACCCTGATATTCCCACCCAGCATGCACTTTTTCCAGGC
CAAGCATCTCATTGAGAGAAGTCAAGTGTTTAATATTCTAAGGATGATGCCAAAA
GGGGCTGCCTTGCACCTCCATGACATTGGCATCGTGACTATGGACTGGCTGGTGA
GGAATGTCACCTACAGGCCTCACTGCCACATCTGTTTCACCCCAAGGGGGATCAT
GCAGTTCAGATTTGCTCACCCAACTCCCCGTCCATCAGAAAAATGTTCCAAGTGG
ATTCTGCTGGAGGATTATCGGAAGCGGGTGCAGAACGTCACTGAGTTTGATGACA
GCTTGCTGAGGAATTTCACTCTGGTGACCCAGCACCCGGAGGTGATTTACACAAA
CCAAAATGTTGTCTGGTCGAAATTTGAAACCATCTTCTTCACCATCTCTGGTCTC
ATCCATTACGCACCAGTGTTCAGAGACTATGTCTTCCGGAGCATGCAGGAGTTCT
ACGAGGACAACGTGCTCTACATGGAGATCAGAGCCCAGCTGCTGCCGGTGTATGA
GCTCAGTGGAGAGCACCATGACGAAGAGTGGTCAGTGAAGACTTACCAGGAAGTA
GCTCAGAAGTTTGTGGAAACTCACCCTGAGTTTATTGGAATCAAAATCATTTATT
CGGATATAAGATCCAAAGATGTGGCTGTCATCGCAGAATCCATCCGAATGGCCAT
GGGGCTCCGAATCAAGTTCCCCACGGTGGTGGCAGGGTTTGACCTGGTGGGGCAT
GAGGACACTGGCCACTCCTTGCATGACTACAAGGAAGCTCTGATGATCCCCGCCA
AGGATGGCGTTAAGCTGCCTTACTTCTTCCACGCCGGAGAAACAGACTGGCAGGG
TACTTCCATAGACAGGAACATTCTGGATGCTCTGATGCTGAACACTACCAGAATC
GGCCATGGATTTGCTTTGAGCAAACACCCCGCAGTCAGGACTTACTCCTGGAAAA
AGGACATCCCCATAGAAGTCTGTCCCATCTCTAACCAGGTGCTGAAACTGGTGTC
TGACTTGAGGAACCACCCTGTAGCCACTCTGATGGCCACTGGGCACCCCATGGTG
ATCAGCTCTGATGACCCAGCTATGTTTGGTGCCAAAGGCTTGTCCTATGATTTCT
ATGAGGTCTTCATGGGCATTGGGGGGATGAAGGCTGACCTGAGGACCCTCAAACA
GCTGGCCATGAACTCTATCAAGTACAGTACCCTGTTGGAGAGTGAGAAAAATACT
TTCATGGAAATCTGGAAGAAGAGATGGGATAAGTTCATAGCAGATGTGGCTACAA
AGGGCGGACACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRAQLLPVYELSGEHHDEEWSVKTYQEVAQK
FVETHPEFIGIKIIYSDIRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2|(R222T, S265A) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCACCCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCAGATCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRATLLPVYELSGEHHDEEWSVKTYQEVAQK
FVETHPEFIGIKIIYADHRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(190F, R222T, L224S, S265A, H301Q) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACTTTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCACCCTGTCACCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCAGATCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDFGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRATLSPVYELSGEHHDEEWSVKTYQEVAQK
FVETHPEFIGIKIIYADHRSKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HSADA2(L87V, 190F, R222T, L224P, S265A, H301Q) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACGTGCATGACTTTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCACCCTGCCCCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCAGATCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCELLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHVHDFGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRATLPPVYELSGEHHDEEWSVKTYQEVAQK
FVETHPEFIGIKIIYADHRSKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(R222T, L224S, S265A) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCACCCTGTCACCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCAGATCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCATGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRATLSPVYELSGEHHDEEWSVKTYQEVAQK
FVETHPEFIGIKIIYADHRSKDVAVIAESIRMAMGLRIKFPTVVAGFDLVGHEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(190F, R222T, L224P, S265A, H301Q) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACTTTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCACCCTGCCCCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCAGATCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDFGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRATLPPVYELSGEHHDEEWSVKTYQEVAQK
FVETHPEFIGIKIIYADHRSKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(R222T, L224S, S265A, H301Q) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCACCCTGTCACCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCAGATCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRATLSPVYELSGEHHDEEWSVKTYQEVAQK
FVETHPEFIGIKIIYADHRSKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(R222T, S265A, H301Q) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCACCCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCAGATCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRATLLPVYELSGEHHDEEWSVKTYQEVAQK
FVETHPEFIGIKIIYADHRSKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(A221S, R222L, S265A, S269A, H301Q) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGATCACTGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCAGATCACA
GAGCAAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRSLLLPVYELSGEHHDEEWSVKTYQEVAQK
FVETHPEFIGIKIIYADHRAKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(A221S, R222L, S265A, S269G, H301Q) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGGGCTGGTGCTGAACACCAAGG
AGGAGCTGGCCAATGAGAGGCTCATGACGCTCAAAATCGCTGAGATGAAGGAGGC
CATGAGGACCCTGATATTCCCACCCAGCATGCACTTTTTCCAGGCCAAGCATCTC
ATTGAGAGAAGTCAAGTGTTTAATATTCTAAGGATGATGCCAAAAGGGGCTGCCT
TGCACCTCCATGACATTGGCATCGTGACTATGGACTGGCTGGTGAGGAATGTCAC
CTACAGGCCTCACTGCCACATCTGTTTCACCCCAAGGGGGATCATGCAGTTCAGA
TTTGCTCACCCAACTCCCCGTCCATCAGAAAAATGTTCCAAGTGGATTCTGCTGG
AGGATTATCGGAAGCGGGTGCAGAACGTCACTGAGTTTGATGACAGCTTGCTGAG
GAATTTCACTCTGGTGACCCAGCACCCGGAGGTGATTTACACAAACCAAAATGTT
GTCTGGTCGAAATTTGAAACCATCTTCTTCACCATCTCTGGTCTCATCCATTACG
CACCAGTGTTCAGAGACTATGTCTTCCGGAGCATGCAGGAGTTCTACGAGGACAA
CGTGCTCTACATGGAGATCAGATCACTCCTGCTGCCGGTGTATGAGCTCAGTGGA
GAGCACCATGACGAAGAGTGGTCAGTGAAGACTTACCAGGAAGTAGCTCAGAAGT
TTGTGGAAACTCACCCTGAGTTTATTGGAATCAAAATCATTTATGCAGATCACAG
AGGCAAAGATGTGGCTGTCATCGCAGAATCCATCCGAATGGCCATGGGGCTCCGA
ATCAAGTTCCCCACGGTGGTGGCAGGGTTTGACCTGGTGGGGCAGGAGGACACTG
GCCACTCCTTGCATGACTACAAGGAAGCTCTGATGATCCCCGCCAAGGATGGCGT
TAAGCTGCCTTACTTCTTCCACGCCGGAGAAACAGACTGGCAGGGTACTTCCATA
GACAGGAACATTCTGGATGCTCTGATGCTGAACACTACCAGAATCGGCCATGGAT
TTGCTTTGAGCAAACACCCCGCAGTCAGGACTTACTCCTGGAAAAAGGACATCCC
CATAGAAGTCTGTCCCATCTCTAACCAGGTGCTGAAACTGGTGTCTGACTTGAGG
AACCACCCTGTAGCCACTCTGATGGCCACTGGGCACCCCATGGTGATCAGCTCTG
ATGACCCAGCTATGTTTGGTGCCAAAGGCTTGTCCTATGATTTCTATGAGGTCTT
CATGGGCATTGGGGGGATGAAGGCTGACCTGAGGACCCTCAAACAGCTGGCCATG
AACTCTATCAAGTACAGTACCCTGTTGGAGAGTGAGAAAAATACTTTCATGGAAA
TCTGGAAGAAGAGATGGGATAAGTTCATAGCAGATGTGGCTACAAAGGGCGGACA
CCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRSLLLPVYELSGEHHDEEWSVKTYQEVAQK
FVETHPEFIGIKIIYADHRGKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(A221T, R222L, S265A, D266P, S269A, H301Q)
with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGGGCTGGTGCTGAACACCAAGG
AGGAGCTGGCCAATGAGAGGCTCATGACGCTCAAAATCGCTGAGATGAAGGAGGC
CATGAGGACCCTGATATTCCCACCCAGCATGCACTTTTTCCAGGCCAAGCATCTC
ATTGAGAGAAGTCAAGTGTTTAATATTCTAAGGATGATGCCAAAAGGGGCTGCCT
TGCACCTCCATGACATTGGCATCGTGACTATGGACTGGCTGGTGAGGAATGTCAC
CTACAGGCCTCACTGCCACATCTGTTTCACCCCAAGGGGGATCATGCAGTTCAGA
TTTGCTCACCCAACTCCCCGTCCATCAGAAAAATGTTCCAAGTGGATTCTGCTGG
AGGATTATCGGAAGCGGGTGCAGAACGTCACTGAGTTTGATGACAGCTTGCTGAG
GAATTTCACTCTGGTGACCCAGCACCCGGAGGTGATTTACACAAACCAAAATGTT
GTCTGGTCGAAATTTGAAACCATCTTCTTCACCATCTCTGGTCTCATCCATTACG
CACCAGTGTTCAGAGACTATGTCTTCCGGAGCATGCAGGAGTTCTACGAGGACAA
CGTGCTCTACATGGAGATCAGAACCCTCCTGCTGCCGGTGTATGAGCTCAGTGGA
GAGCACCATGACGAAGAGTGGTCAGTGAAGACTTACCAGGAAGTAGCTCAGAAGT
TTGTGGAAACTCACCCTGAGTTTATTGGAATCAAAATCATTTATGCACCCCACAG
AGCAAAAGATGTGGCTGTCATCGCAGAATCCATCCGAATGGCCATGGGGCTCCGA
ATCAAGTTCCCCACGGTGGTGGCAGGGTTTGACCTGGTGGGGCAGGAGGACACTG
GCCACTCCTTGCATGACTACAAGGAAGCTCTGATGATCCCCGCCAAGGATGGCGT
TAAGCTGCCTTACTTCTTCCACGCCGGAGAAACAGACTGGCAGGGTACTTCCATA
GACAGGAACATTCTGGATGCTCTGATGCTGAACACTACCAGAATCGGCCATGGAT
TTGCTTTGAGCAAACACCCCGCAGTCAGGACTTACTCCTGGAAAAAGGACATCCC
CATAGAAGTCTGTCCCATCTCTAACCAGGTGCTGAAACTGGTGTCTGACTTGAGG
AACCACCCTGTAGCCACTCTGATGGCCACTGGGCACCCCATGGTGATCAGCTCTG
ATGACCCAGCTATGTTTGGTGCCAAAGGCTTGTCCTATGATTTCTATGAGGTCTT
CATGGGCATTGGGGGGATGAAGGCTGACCTGAGGACCCTCAAACAGCTGGCCATG
AACTCTATCAAGTACAGTACCCTGTTGGAGAGTGAGAAAAATACTTTCATGGAAA
TCTGGAAGAAGAGATGGGATAAGTTCATAGCAGATGTGGCTACAAAGGGCGGACA
CCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRTLLLPVYELSGEHHDEEWSVKTYQEVAQK
FVETHPEFIGIKIIYAPHRAKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(A221T, R222I, S265A, D266P, S269A, H301Q)
with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAACCATACTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCACCCCACA
GAGCAAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRTILLPVYELSGEHHDEEWSVKTYQEVAQK
FVETHPEFIGIKIIYAPHRAKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(A221S, R222I, S265A, D266P, S269A, H301Q) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGATCAATACTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCACCCCACA
GAGCAAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRSILLPVYELSGEHHDEEWSVKTYQEVAQK
FVETHPEFIGIKIIYAPHRAKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(A221T, R222L, S265A, D266P, S269M, H301Q)
with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAACCCTGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCACCCCACA
GAATGAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRTLLLPVYELSGEHHDEEWSVKTYQEVAQK
FVETHPEFIGIKIIYAPHRMKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(A221S, R222L, S265A, D266P, S269M, H301Q)
with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGATCACTGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCACCCCACA
GAATGAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLG
GRLVLNTKEELANERLMTLKIAEMKEAMRTLIFPPSMHFFQAKHLIERSQVFNIL
RMMPKGAALHLHDIGIVTMDWLVRNVTYRPHCHICFTPRGIMQFRFAHPTPRPSE
KCSKWILLEDYRKRVQNVTEFDDSLLRNFTLVTQHPEVIYTNQNVVWSKFETIFF
TISGLIHYAPVFRDYVFRSMQEFYEDNVLYMEIRSLLLPVYELSGEHHDEEWSVK
TYQEVAQKFVETHPEFIGIKIIYAPHRMKDVAVIAESIRMAMGLRIKFPTVVAGF
DLVGQEDTGHSLHDYKEALMIPAKDGVKLPYFFHAGETDWQGTSIDRNILDALML
NTTRIGHGFALSKHPAVRTYSWKKDIPIEVCPISNQVLKLVSDLRNHPVATLMAT
GHPMVISSDDPAMFGAKGLSYDFYEVFMGIGGMKADLRTLKQLAMNSIKYSTLLE
SEKNTFMEIWKKRWDKFIADVATKGGHHHHHHGG
>HsADA2(A221S, R222L, S265A, D266P, S269G, H301Q)
with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGATCACTGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCACCCCACA
GAGGCAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLG
GRLVLNTKEELANERLMTLKIAEMKEAMRTLIFPPSMHFFQAKHLIERSQVFNIL
RMMPKGAALHLHDIGIVTMDWLVRNVTYRPHCHICFTPRGIMQFRFAHPTPRPSE
KCSKWILLEDYRKRVQNVTEFDDSLLRNFTLVTQHPEVIYTNQNVVWSKFETIFF
TISGLIHYAPVFRDYVFRSMQEFYEDNVLYMEIRSLLLPVYELSGEHHDEEWSVK
TYQEVAQKFVETHPEFIGIKIIYAPHRGKDVAVIAESIRMAMGLRIKFPTVVAGF
DLVGQEDTGHSLHDYKEALMIPAKDGVKLPYFFHAGETDWQGTSIDRNILDALML
NTTRIGHGFALSKHPAVRTYSWKKDIPIEVCPISNQVLKLVSDLRNHPVATLMAT
GHPMVISSDDPAMFGAKGLSYDFYEVFMGIGGMKADLRTLKQLAMNSIKYSTLLE
SEKNTFMEIWKKRWDKFIADVATKGGHHHHHHGG
>HsADA2(A221S, R2221, S265A, D266P, S269G, H301Q)
with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGATCAATACTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCACCCCACA
GAGGCAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRSILLPVYELSGEHHDEEWSVKTYQEVAQK
FVETHPEFIGIKIIYAPHRGKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(R222T, L224S, S265A, D266P, H301Q) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCACCCTGTCACCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCACCCCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRATLSPVYELSGEHHDEEWSVKTYQEVAQK
FVETHPEFIGIKIIYAPHRSKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(R222M, S265A, D266P, S269M, H301Q) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAGCCATGCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCACCCCACA
GAATGAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRAMLLPVYELSGEHHDEEWSVKTYQEVAQK
FVETHPEFIGIKIIYAPHRMKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(A221T, R222T, S265A, D266P, H301Q) with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAACCACCCTGCTGCCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCACCCCACA
GATCCAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRTTLLPVYELSGEHHDEEWSVKTYQEVAQK
FVETHPEFIGIKIIYAPHRSKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2
(A221S, R222L, L224S, S265A, D266P, S269M, H301Q) with
6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGATCACTGCTGTCACCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCACCCCACA
GAATGAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRSLLSPVYELSGEHHDEEWSVKTYQEVAQK
FVETHPEFIGIKIIYAPHRMKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>HsADA2(A221T, R222L, S265A, D266P, S269M, H301Q)
with 6xHis tag
(Seq ID No. .)
ATGTTGGTGGATGGCCCATCTGAGCGGCCAGCCCTGTGCTTCTTGCTGTTGGCTG
TGGCAATGTCTTTCTTCGGCTCAGCTCTATCCATAGATGAAACACGGGCGCATCT
GTTGTTGAAAGAAAAGATGATGCGGCTGGGGGGGCGGCTGGTGCTGAACACCAAG
GAGGAGCTGGCCAATGAGAGGCTCATGACGCTCAAAATCGCTGAGATGAAGGAGG
CCATGAGGACCCTGATATTCCCACCCAGCATGCACTTTTTCCAGGCCAAGCATCT
CATTGAGAGAAGTCAAGTGTTTAATATTCTAAGGATGATGCCAAAAGGGGCTGCC
TTGCACCTCCATGACATTGGCATCGTGACTATGGACTGGCTGGTGAGGAATGTCA
CCTACAGGCCTCACTGCCACATCTGTTTCACCCCAAGGGGGATCATGCAGTTCAG
ATTTGCTCACCCAACTCCCCGTCCATCAGAAAAATGTTCCAAGTGGATTCTGCTG
GAGGATTATCGGAAGCGGGTGCAGAACGTCACTGAGTTTGATGACAGCTTGCTGA
GGAATTTCACTCTGGTGACCCAGCACCCGGAGGTGATTTACACAAACCAAAATGT
TGTCTGGTCGAAATTTGAAACCATCTTCTTCACCATCTCTGGTCTCATCCATTAC
GCACCAGTGTTCAGAGACTATGTCTTCCGGAGCATGCAGGAGTTCTACGAGGACA
ACGTGCTCTACATGGAGATCAGAACCCTGCTGTCACCGGTGTATGAGCTCAGTGG
AGAGCACCATGACGAAGAGTGGTCAGTGAAGACTTACCAGGAAGTAGCTCAGAAG
TTTGTGGAAACTCACCCTGAGTTTATTGGAATCAAAATCATTTATGCACCCCACA
GAATGAAAGATGTGGCTGTCATCGCAGAATCCATCCGAATGGCCATGGGGCTCCG
AATCAAGTTCCCCACGGTGGTGGCAGGGTTTGACCTGGTGGGGCAGGAGGACACT
GGCCACTCCTTGCATGACTACAAGGAAGCTCTGATGATCCCCGCCAAGGATGGCG
TTAAGCTGCCTTACTTCTTCCACGCCGGAGAAACAGACTGGCAGGGTACTTCCAT
AGACAGGAACATTCTGGATGCTCTGATGCTGAACACTACCAGAATCGGCCATGGA
TTTGCTTTGAGCAAACACCCCGCAGTCAGGACTTACTCCTGGAAAAAGGACATCC
CCATAGAAGTCTGTCCCATCTCTAACCAGGTGCTGAAACTGGTGTCTGACTTGAG
GAACCACCCTGTAGCCACTCTGATGGCCACTGGGCACCCCATGGTGATCAGCTCT
GATGACCCAGCTATGTTTGGTGCCAAAGGCTTGTCCTATGATTTCTATGAGGTCT
TCATGGGCATTGGGGGGATGAAGGCTGACCTGAGGACCCTCAAACAGCTGGCCAT
GAACTCTATCAAGTACAGTACCCTGTTGGAGAGTGAGAAAAATACTTTCATGGAA
ATCTGGAAGAAGAGATGGGATAAGTTCATAGCAGATGTGGCTACAAAGGGCGGAC
ACCACCATCACCACCATGGAGGCTAG
(Seq ID No. .)
MLVDGPSERPALCFLLLAVAMSFFGSALSIDETRAHLLLKEKMMRLGGRLVLNTK
EELANERLMTLKIAEMKEAMRTLIFPPSMHFFQAKHLIERSQVFNILRMMPKGAA
LHLHDIGIVTMDWLVRNVTYRPHCHICFTPRGIMQFRFAHPTPRPSEKCSKWILL
EDYRKRVQNVTEFDDSLLRNFTLVTQHPEVIYTNQNVVWSKFETIFFTISGLIHY
APVFRDYVFRSMQEFYEDNVLYMEIRTLLSPVYELSGEHHDEEWSVKTYQEVAQK
FVETHPEFIGIKIIYAPHRMKDVAVIAESIRMAMGLRIKFPTVVAGFDLVGQEDT
GHSLHDYKEALMIPAKDGVKLPYFFHAGETDWQGTSIDRNILDALMLNTTRIGHG
FALSKHPAVRTYSWKKDIPIEVCPISNQVLKLVSDLRNHPVATLMATGHPMVISS
DDPAMFGAKGLSYDFYEVFMGIGGMKADLRTLKQLAMNSIKYSTLLESEKNTFME
IWKKRWDKFIADVATKGGHHHHHHGG
>IgKCm SP with 3′ Gln/Q overhang
(Seq ID No. .)
ATGGGCAGCGCCGCTCTCCTGCTCTGGGTGCTGCTGCTGTGGGTGCCCAGCTCCA
GGGCCCAG
(Seq ID No. .)
MGSAALLLWVLLLWVPSSRAQ
>GRZMB SP with 3′ Gln/Q overhang
(Seq ID No. .)
ATGCAACCAATCCTGCTTCTGCTGGCCTTCCTCCTGCTGCCCAGGGCAGATGCAG
(Seq ID No. .)
MQPILLLLAFLLLPRADAQ
>CD177 SP with 3′ Gln/Q overhang
(Seq ID No. .)
ATGAGCGCGGTATTACTGCTGGCCCTCCTGGGGTTCATCCTCCCACTGCCAGGAG
TGCAGGCGCAG
(Seq ID No. .)
MSAVLLLALLGFILPLPGVQAQ
>SA SP with 3′ Gln/Q overhang
(Seq ID No. .)
ATGAAGTGGGTAACCTTTATTTCCCTTCTTTTTCTCTTTAGCTCGGCTTATTCCC
AG
(Seq ID No. .)
MKWVTFISLLFLFSSAYSQ
>IL-2 SP with 3′ Gln/Q overhang
(Seq ID No. .)
ATGTACAGGATGCAACTCCTGTCTTGCATTGCACTAAGTCTTGCACTTGTCACAA
ACAGTCAG
(Seq ID No. .)
MYRMQLLSCIALSLALVTNSQ
>PROS SP with 3′ Gln/Q overhang
(Seq ID No. .)
ATGAGGGTCCTGGGTGGGCGCTGCGGGGCGCTGCTGGCGTGTCTCCTCCTAGTGC
TTCCCGTGTCAGAGGCAGCCCAG
(Seq ID No. .)
MRVLGGRCGALLACLLLVLPVSEAAQ
>PROS-HsADA2-(R222T, S265A)|PROS SP with HsADA2
(R222T, S265A) and 6xHis tag
(Seq ID No. .)
ATGAGGGTCCTGGGTGGGCGCTGCGGGGCGCTGCTGGCGTGTCTCCTCCTAGTGC
TTCCCGTGTCAGAGGCAGCCCAGATAGATGAAACACGGGCGCATCTGTTGTTGAA
AGAAAAGATGATGCGGCTGGGGGGGCGGCTGGTGCTGAACACCAAGGAGGAGCTG
GCCAATGAGAGGCTCATGACGCTCAAAATCGCTGAGATGAAGGAGGCCATGAGGA
CCCTGATATTCCCACCCAGCATGCACTTTTTCCAGGCCAAGCATCTCATTGAGAG
AAGTCAAGTGTTTAATATTCTAAGGATGATGCCAAAAGGGGCTGCCTTGCACCTC
CATGACATTGGCATCGTGACTATGGACTGGCTGGTGAGGAATGTCACCTACAGGC
CTCACTGCCACATCTGTTTCACCCCAAGGGGGATCATGCAGTTCAGATTTGCTCA
CCCAACTCCCCGTCCATCAGAAAAATGTTCCAAGTGGATTCTGCTGGAGGATTAT
CGGAAGCGGGTGCAGAACGTCACTGAGTTTGATGACAGCTTGCTGAGGAATTTCA
CTCTGGTGACCCAGCACCCGGAGGTGATTTACACAAACCAAAATGTTGTCTGGTC
GAAATTTGAAACCATCTTCTTCACCATCTCTGGTCTCATCCATTACGCACCAGTG
TTCAGAGACTATGTCTTCCGGAGCATGCAGGAGTTCTACGAGGACAACGTGCTCT
ACATGGAGATCAGAGCCACCCTGCTGCCGGTGTATGAGCTCAGTGGAGAGCACCA
TGACGAAGAGTGGTCAGTGAAGACTTACCAGGAAGTAGCTCAGAAGTTTGTGGAA
ACTCACCCTGAGTTTATTGGAATCAAAATCATTTATGCAGATCACAGATCCAAAG
ATGTGGCTGTCATCGCAGAATCCATCCGAATGGCCATGGGGCTCCGAATCAAGTT
CCCCACGGTGGTGGCAGGGTTTGACCTGGTGGGGCATGAGGACACTGGCCACTCC
TTGCATGACTACAAGGAAGCTCTGATGATCCCCGCCAAGGATGGCGTTAAGCTGC
CTTACTTCTTCCACGCCGGAGAAACAGACTGGCAGGGTACTTCCATAGACAGGAA
CATTCTGGATGCTCTGATGCTGAACACTACCAGAATCGGCCATGGATTTGCTTTG
AGCAAACACCCCGCAGTCAGGACTTACTCCTGGAAAAAGGACATCCCCATAGAAG
TCTGTCCCATCTCTAACCAGGTGCTGAAACTGGTGTCTGACTTGAGGAACCACCC
TGTAGCCACTCTGATGGCCACTGGGCACCCCATGGTGATCAGCTCTGATGACCCA
GCTATGTTTGGTGCCAAAGGCTTGTCCTATGATTTCTATGAGGTCTTCATGGGCA
TTGGGGGGATGAAGGCTGACCTGAGGACCCTCAAACAGCTGGCCATGAACTCTAT
CAAGTACAGTACCCTGTTGGAGAGTGAGAAAAATACTTTCATGGAAATCTGGAAG
AAGAGATGGGATAAGTTCATAGCAGATGTGGCTACAAAGGGCGGACACCACCATC
ACCACCATGGAGGCTAG
(Seq ID No. .)
MRVLGGRCGALLACLLLVLPVSEAAQIDETRAHLLLKEKMMRLGGRLVLNTKEEL
ANERLMTLKIAEMKEAMRTLIFPPSMHFFQAKHLIERSQVFNILRMMPKGAALHL
HDIGIVTMDWLVRNVTYRPHCHICFTPRGIMQFRFAHPTPRPSEKCSKWILLEDY
RKRVQNVTEFDDSLLRNFTLVTQHPEVIYTNQNVVWSKFETIFFTISGLIHYAPV
FRDYVFRSMQEFYEDNVLYMEIRATLLPVYELSGEHHDEEWSVKTYQEVAQKFVE
THPEFIGIKIIYADHRSKDVAVIAESIRMAMGLRIKFPTVVAGFDLVGHEDTGHS
LHDYKEALMIPAKDGVKLPYFFHAGETDWQGTSIDRNILDALMLNTTRIGHGFAL
SKHPAVRTYSWKKDIPIEVCPISNQVLKLVSDLRNHPVATLMATGHPMVISSDDP
AMFGAKGLSYDFYEVFMGIGGMKADLRTLKQLAMNSIKYSTLLESEKNTFMEIWK
KRWDKFIADVATKGGHHHHHHGG
>IgKCm-HsADA2-(R222T, S265A)|IgKCm SP with HsADA2
(R222T, S265A) and 6xHis tag
(Seq ID No. .)
ATGGGCAGCGCCGCTCTCCTGCTCTGGGTGCTGCTGCTGTGGGTGCCCAGCTCCA
GGGCCCAGATAGATGAAACACGGGCGCATCTGTTGTTGAAAGAAAAGATGATGCG
GCTGGGGGGGCGGCTGGTGCTGAACACCAAGGAGGAGCTGGCCAATGAGAGGCTC
ATGACGCTCAAAATCGCTGAGATGAAGGAGGCCATGAGGACCCTGATATTCCCAC
CCAGCATGCACTTTTTCCAGGCCAAGCATCTCATTGAGAGAAGTCAAGTGTTTAA
TATTCTAAGGATGATGCCAAAAGGGGCTGCCTTGCACCTCCATGACATTGGCATC
GTGACTATGGACTGGCTGGTGAGGAATGTCACCTACAGGCCTCACTGCCACATCT
GTTTCACCCCAAGGGGGATCATGCAGTTCAGATTTGCTCACCCAACTCCCCGTCC
ATCAGAAAAATGTTCCAAGTGGATTCTGCTGGAGGATTATCGGAAGCGGGTGCAG
AACGTCACTGAGTTTGATGACAGCTTGCTGAGGAATTTCACTCTGGTGACCCAGC
ACCCGGAGGTGATTTACACAAACCAAAATGTTGTCTGGTCGAAATTTGAAACCAT
CTTCTTCACCATCTCTGGTCTCATCCATTACGCACCAGTGTTCAGAGACTATGTC
TTCCGGAGCATGCAGGAGTTCTACGAGGACAACGTGCTCTACATGGAGATCAGAG
CCACCCTGCTGCCGGTGTATGAGCTCAGTGGAGAGCACCATGACGAAGAGTGGTC
AGTGAAGACTTACCAGGAAGTAGCTCAGAAGTTTGTGGAAACTCACCCTGAGTTT
ATTGGAATCAAAATCATTTATGCAGATCACAGATCCAAAGATGTGGCTGTCATCG
CAGAATCCATCCGAATGGCCATGGGGCTCCGAATCAAGTTCCCCACGGTGGTGGC
AGGGTTTGACCTGGTGGGGCATGAGGACACTGGCCACTCCTTGCATGACTACAAG
GAAGCTCTGATGATCCCCGCCAAGGATGGCGTTAAGCTGCCTTACTTCTTCCACG
CCGGAGAAACAGACTGGCAGGGTACTTCCATAGACAGGAACATTCTGGATGCTCT
GATGCTGAACACTACCAGAATCGGCCATGGATTTGCTTTGAGCAAACACCCCGCA
GTCAGGACTTACTCCTGGAAAAAGGACATCCCCATAGAAGTCTGTCCCATCTCTA
ACCAGGTGCTGAAACTGGTGTCTGACTTGAGGAACCACCCTGTAGCCACTCTGAT
GGCCACTGGGCACCCCATGGTGATCAGCTCTGATGACCCAGCTATGTTTGGTGCC
AAAGGCTTGTCCTATGATTTCTATGAGGTCTTCATGGGCATTGGGGGGATGAAGG
CTGACCTGAGGACCCTCAAACAGCTGGCCATGAACTCTATCAAGTACAGTACCCT
GTTGGAGAGTGAGAAAAATACTTTCATGGAAATCTGGAAGAAGAGATGGGATAAG
TTCATAGCAGATGTGGCTACAAAGGGCGGACACCACCATCACCACCATGGAGGCT
AG
(Seq ID No. .)
MGSAALLLWVLLLWVPSSRAQIDETRAHLLLKEKMMRLGGRLVLNTKEELANERL
MTLKIAEMKEAMRTLIFPPSMHFFQAKHLIERSQVFNILRMMPKGAALHLHDIGI
VTMDWLVRNVTYRPHCHICFTPRGIMQFRFAHPTPRPSEKCSKWILLEDYRKRVQ
NVTEFDDSLLRNFTLVTQHPEVIYTNQNVVWSKFETIFFTISGLIHYAPVFRDYV
FRSMQEFYEDNVLYMEIRATLLPVYELSGEHHDEEWSVKTYQEVAQKFVETHPEF
IGIKIIYADHRSKDVAVIAESIRMAMGLRIKFPTVVAGFDLVGHEDTGHSLHDYK
EALMIPAKDGVKLPYFFHAGETDWQGTSIDRNILDALMLNTTRIGHGFALSKHPA
VRTYSWKKDIPIEVCPISNQVLKLVSDLRNHPVATLMATGHPMVISSDDPAMFGA
KGLSYDFYEVFMGIGGMKADLRTLKQLAMNSIKYSTLLESEKNTFMEIWKKRWDK
FIADVATKGGHHHHHHGG
>GRZMB-HsADA2-(R222T, S265A)|GRZMB SP with HsADA2
(R222T, S265A) and 6xHis tag
(Seq ID No. .)
ATGCAACCAATCCTGCTTCTGCTGGCCTTCCTCCTGCTGCCCAGGGCAGATGCAG
ATAGATGAAACACGGGCGCATCTGTTGTTGAAAGAAAAGATGATGCGGCTGGGGG
GGCGGCTGGTGCTGAACACCAAGGAGGAGCTGGCCAATGAGAGGCTCATGACGCT
CAAAATCGCTGAGATGAAGGAGGCCATGAGGACCCTGATATTCCCACCCAGCATG
CACTTTTTCCAGGCCAAGCATCTCATTGAGAGAAGTCAAGTGTTTAATATTCTAA
GGATGATGCCAAAAGGGGCTGCCTTGCACCTCCATGACATTGGCATCGTGACTAT
GGACTGGCTGGTGAGGAATGTCACCTACAGGCCTCACTGCCACATCTGTTTCACC
CCAAGGGGGATCATGCAGTTCAGATTTGCTCACCCAACTCCCCGTCCATCAGAAA
AATGTTCCAAGTGGATTCTGCTGGAGGATTATCGGAAGCGGGTGCAGAACGTCAC
TGAGTTTGATGACAGCTTGCTGAGGAATTTCACTCTGGTGACCCAGCACCCGGAG
GTGATTTACACAAACCAAAATGTTGTCTGGTCGAAATTTGAAACCATCTTCTTCA
CCATCTCTGGTCTCATCCATTACGCACCAGTGTTCAGAGACTATGTCTTCCGGAG
CATGCAGGAGTTCTACGAGGACAACGTGCTCTACATGGAGATCAGAGCCACCCTG
CTGCCGGTGTATGAGCTCAGTGGAGAGCACCATGACGAAGAGTGGTCAGTGAAGA
CTTACCAGGAAGTAGCTCAGAAGTTTGTGGAAACTCACCCTGAGTTTATTGGAAT
CAAAATCATTTATGCAGATCACAGATCCAAAGATGTGGCTGTCATCGCAGAATCC
ATCCGAATGGCCATGGGGCTCCGAATCAAGTTCCCCACGGTGGTGGCAGGGTTTG
ACCTGGTGGGGCATGAGGACACTGGCCACTCCTTGCATGACTACAAGGAAGCTCT
GATGATCCCCGCCAAGGATGGCGTTAAGCTGCCTTACTTCTTCCACGCCGGAGAA
ACAGACTGGCAGGGTACTTCCATAGACAGGAACATTCTGGATGCTCTGATGCTGA
ACACTACCAGAATCGGCCATGGATTTGCTTTGAGCAAACACCCCGCAGTCAGGAC
TTACTCCTGGAAAAAGGACATCCCCATAGAAGTCTGTCCCATCTCTAACCAGGTG
CTGAAACTGGTGTCTGACTTGAGGAACCACCCTGTAGCCACTCTGATGGCCACTG
GGCACCCCATGGTGATCAGCTCTGATGACCCAGCTATGTTTGGTGCCAAAGGCTT
GTCCTATGATTTCTATGAGGTCTTCATGGGCATTGGGGGGATGAAGGCTGACCTG
AGGACCCTCAAACAGCTGGCCATGAACTCTATCAAGTACAGTACCCTGTTGGAGA
GTGAGAAAAATACTTTCATGGAAATCTGGAAGAAGAGATGGGATAAGTTCATAGC
AGATGTGGCTACAAAGGGCGGACACCACCATCACCACCATGGAGGCTAG
(Seq ID No. .)
MQPILLLLAFLLLPRADAQIDETRAHLLLKEKMMRLGGRLVLNTKEELANERLMT
LKIAEMKEAMRTLIFPPSMHFFQAKHLIERSQVFNILRMMPKGAALHLHDIGIVT
MDWLVRNVTYRPHCHICFTPRGIMQFRFAHPTPRPSEKCSKWILLEDYRKRVQNV
TEFDDSLLRNFTLVTQHPEVIYTNQNVVWSKFETIFFTISGLIHYAPVFRDYVFR
SMQEFYEDNVLYMEIRATLLPVYELSGEHHDEEWSVKTYQEVAQKFVETHPEFIG
IKIIYADHRSKDVAVIAESIRMAMGLRIKFPTVVAGFDLVGHEDTGHSLHDYKEA
LMIPAKDGVKLPYFFHAGETDWQGTSIDRNILDALMLNTTRIGHGFALSKHPAVR
TYSWKKDIPIEVCPISNQVLKLVSDLRNHPVATLMATGHPMVISSDDPAMFGAKG
LSYDFYEVFMGIGGMKADLRTLKQLAMNSIKYSTLLESEKNTFMEIWKKRWDKFI
ADVATKGGHHHHHHGG
>CD177-HsADA2-(R222T, S265A)|CD177 SP with HsADA2
(R222T, S265A) and 6xHis tag
(Seq ID No. .)
ATGAGCGCGGTATTACTGCTGGCCCTCCTGGGGTTCATCCTCCCACTGCCAGGAG
TGCAGGCGCAGATAGATGAAACACGGGCGCATCTGTTGTTGAAAGAAAAGATGAT
GCGGCTGGGGGGGCGGCTGGTGCTGAACACCAAGGAGGAGCTGGCCAATGAGAGG
CTCATGACGCTCAAAATCGCTGAGATGAAGGAGGCCATGAGGACCCTGATATTCC
CACCCAGCATGCACTTTTTCCAGGCCAAGCATCTCATTGAGAGAAGTCAAGTGTT
TAATATTCTAAGGATGATGCCAAAAGGGGCTGCCTTGCACCTCCATGACATTGGC
ATCGTGACTATGGACTGGCTGGTGAGGAATGTCACCTACAGGCCTCACTGCCACA
TCTGTTTCACCCCAAGGGGGATCATGCAGTTCAGATTTGCTCACCCAACTCCCCG
TCCATCAGAAAAATGTTCCAAGTGGATTCTGCTGGAGGATTATCGGAAGCGGGTG
CAGAACGTCACTGAGTTTGATGACAGCTTGCTGAGGAATTTCACTCTGGTGACCC
AGCACCCGGAGGTGATTTACACAAACCAAAATGTTGTCTGGTCGAAATTTGAAAC
CATCTTCTTCACCATCTCTGGTCTCATCCATTACGCACCAGTGTTCAGAGACTAT
GTCTTCCGGAGCATGCAGGAGTTCTACGAGGACAACGTGCTCTACATGGAGATCA
GAGCCACCCTGCTGCCGGTGTATGAGCTCAGTGGAGAGCACCATGACGAAGAGTG
GTCAGTGAAGACTTACCAGGAAGTAGCTCAGAAGTTTGTGGAAACTCACCCTGAG
TTTATTGGAATCAAAATCATTTATGCAGATCACAGATCCAAAGATGTGGCTGTCA
TCGCAGAATCCATCCGAATGGCCATGGGGCTCCGAATCAAGTTCCCCACGGTGGT
GGCAGGGTTTGACCTGGTGGGGCATGAGGACACTGGCCACTCCTTGCATGACTAC
AAGGAAGCTCTGATGATCCCCGCCAAGGATGGCGTTAAGCTGCCTTACTTCTTCC
ACGCCGGAGAAACAGACTGGCAGGGTACTTCCATAGACAGGAACATTCTGGATGC
TCTGATGCTGAACACTACCAGAATCGGCCATGGATTTGCTTTGAGCAAACACCCC
GCAGTCAGGACTTACTCCTGGAAAAAGGACATCCCCATAGAAGTCTGTCCCATCT
CTAACCAGGTGCTGAAACTGGTGTCTGACTTGAGGAACCACCCTGTAGCCACTCT
GATGGCCACTGGGCACCCCATGGTGATCAGCTCTGATGACCCAGCTATGTTTGGT
GCCAAAGGCTTGTCCTATGATTTCTATGAGGTCTTCATGGGCATTGGGGGGATGA
AGGCTGACCTGAGGACCCTCAAACAGCTGGCCATGAACTCTATCAAGTACAGTAC
CCTGTTGGAGAGTGAGAAAAATACTTTCATGGAAATCTGGAAGAAGAGATGGGAT
AAGTTCATAGCAGATGTGGCTACAAAGGGCGGACACCACCATCACCACCATGGAG
GCTAG
(Seq ID No. .)
MSAVLLLALLGFILPLPGVQAQIDETRAHLLLKEKMMRLGGRLVLNTKEELANER
LMTLKIAEMKEAMRTLIFPPSMHFFQAKHLIERSQVFNILRMMPKGAALHLHDIG
IVTMDWLVRNVTYRPHCHICFTPRGIMQFRFAHPTPRPSEKCSKWILLEDYRKRV
QNVTEFDDSLLRNFTLVTQHPEVIYTNQNVVWSKFETIFFTISGLIHYAPVFRDY
VFRSMQEFYEDNVLYMEIRATLLPVYELSGEHHDEEWSVKTYQEVAQKFVETHPE
FIGIKIIYADHRSKDVAVIAESIRMAMGLRIKFPTVVAGFDLVGHEDTGHSLHDY
KEALMIPAKDGVKLPYFFHAGETDWQGTSIDRNILDALMLNTTRIGHGFALSKHP
AVRTYSWKKDIPIEVCPISNQVLKLVSDLRNHPVATLMATGHPMVISSDDPAMFG
AKGLSYDFYEVFMGIGGMKADLRTLKQLAMNSIKYSTLLESEKNTFMEIWKKRWD
KFIADVATKGGHHHHHHGG
>SA-HsADA2-(R222T, S265A)|SA SP with HsADA2
(R222T, S265A) and 6xHis tag
(Seq ID No. .)
ATGAAGTGGGTAACCTTTATTTCCCTTCTTTTTCTCTTTAGCTCGGCTTATTCCC
AGATAGATGAAACACGGGCGCATCTGTTGTTGAAAGAAAAGATGATGCGGCTGGG
GGGGCGGCTGGTGCTGAACACCAAGGAGGAGCTGGCCAATGAGAGGCTCATGACG
CTCAAAATCGCTGAGATGAAGGAGGCCATGAGGACCCTGATATTCCCACCCAGCA
TGCACTTTTTCCAGGCCAAGCATCTCATTGAGAGAAGTCAAGTGTTTAATATTCT
AAGGATGATGCCAAAAGGGGCTGCCTTGCACCTCCATGACATTGGCATCGTGACT
ATGGACTGGCTGGTGAGGAATGTCACCTACAGGCCTCACTGCCACATCTGTTTCA
CCCCAAGGGGGATCATGCAGTTCAGATTTGCTCACCCAACTCCCCGTCCATCAGA
AAAATGTTCCAAGTGGATTCTGCTGGAGGATTATCGGAAGCGGGTGCAGAACGTC
ACTGAGTTTGATGACAGCTTGCTGAGGAATTTCACTCTGGTGACCCAGCACCCGG
AGGTGATTTACACAAACCAAAATGTTGTCTGGTCGAAATTTGAAACCATCTTCTT
CACCATCTCTGGTCTCATCCATTACGCACCAGTGTTCAGAGACTATGTCTTCCGG
AGCATGCAGGAGTTCTACGAGGACAACGTGCTCTACATGGAGATCAGAGCCACCC
TGCTGCCGGTGTATGAGCTCAGTGGAGAGCACCATGACGAAGAGTGGTCAGTGAA
GACTTACCAGGAAGTAGCTCAGAAGTTTGTGGAAACTCACCCTGAGTTTATTGGA
ATCAAAATCATTTATGCAGATCACAGATCCAAAGATGTGGCTGTCATCGCAGAAT
CCATCCGAATGGCCATGGGGCTCCGAATCAAGTTCCCCACGGTGGTGGCAGGGTT
TGACCTGGTGGGGCATGAGGACACTGGCCACTCCTTGCATGACTACAAGGAAGCT
CTGATGATCCCCGCCAAGGATGGCGTTAAGCTGCCTTACTTCTTCCACGCCGGAG
AAACAGACTGGCAGGGTACTTCCATAGACAGGAACATTCTGGATGCTCTGATGCT
GAACACTACCAGAATCGGCCATGGATTTGCTTTGAGCAAACACCCCGCAGTCAGG
ACTTACTCCTGGAAAAAGGACATCCCCATAGAAGTCTGTCCCATCTCTAACCAGG
TGCTGAAACTGGTGTCTGACTTGAGGAACCACCCTGTAGCCACTCTGATGGCCAC
TGGGCACCCCATGGTGATCAGCTCTGATGACCCAGCTATGTTTGGTGCCAAAGGC
TTGTCCTATGATTTCTATGAGGTCTTCATGGGCATTGGGGGGATGAAGGCTGACC
TGAGGACCCTCAAACAGCTGGCCATGAACTCTATCAAGTACAGTACCCTGTTGGA
GAGTGAGAAAAATACTTTCATGGAAATCTGGAAGAAGAGATGGGATAAGTTCATA
GCAGATGTGGCTACAAAGGGCGGACACCACCATCACCACCATGGAGGCTAG
(Seq ID No. .)
MKWVTFISLLFLESSAYSQIDETRAHLLLKEKMMRLGGRLVLNTKEELANERLMT
LKIAEMKEAMRTLIFPPSMHFFQAKHLIERSQVFNILRMMPKGAALHLHDIGIVT
MDWLVRNVTYRPHCHICFTPRGIMQFRFAHPTPRPSEKCSKWILLEDYRKRVQNV
TEFDDSLLRNFTLVTQHPEVIYTNQNVVWSKFETIFFTISGLIHYAPVFRDYVFR
SMQEFYEDNVLYMEIRATLLPVYELSGEHHDEEWSVKTYQEVAQKFVETHPEFIG
IKIIYADHRSKDVAVIAESIRMAMGLRIKFPTVVAGFDLVGHEDTGHSLHDYKEA
LMIPAKDGVKLPYFFHAGETDWQGTSIDRNILDALMLNTTRIGHGFALSKHPAVR
TYSWKKDIPIEVCPISNQVLKLVSDLRNHPVATLMATGHPMVISSDDPAMFGAKG
LSYDFYEVFMGIGGMKADLRTLKQLAMNSIKYSTLLESEKNTFMEIWKKRWDKFI
ADVATKGGHHHHHHGG
>IL-2-HsADA2-(R222T, S265A)|IL-2 SP with HsADA2
(R222T, S265A) and 6xHis tag
(Seq ID No. .)
ATGTACAGGATGCAACTCCTGTCTTGCATTGCACTAAGTCTTGCACTTGTCACAA
ACAGTCAGATAGATGAAACACGGGCGCATCTGTTGTTGAAAGAAAAGATGATGCG
GCTGGGGGGGCGGCTGGTGCTGAACACCAAGGAGGAGCTGGCCAATGAGAGGCTC
ATGACGCTCAAAATCGCTGAGATGAAGGAGGCCATGAGGACCCTGATATTCCCAC
CCAGCATGCACTTTTTCCAGGCCAAGCATCTCATTGAGAGAAGTCAAGTGTTTAA
TATTCTAAGGATGATGCCAAAAGGGGCTGCCTTGCACCTCCATGACATTGGCATC
GTGACTATGGACTGGCTGGTGAGGAATGTCACCTACAGGCCTCACTGCCACATCT
GTTTCACCCCAAGGGGGATCATGCAGTTCAGATTTGCTCACCCAACTCCCCGTCC
ATCAGAAAAATGTTCCAAGTGGATTCTGCTGGAGGATTATCGGAAGCGGGTGCAG
AACGTCACTGAGTTTGATGACAGCTTGCTGAGGAATTTCACTCTGGTGACCCAGC
ACCCGGAGGTGATTTACACAAACCAAAATGTTGTCTGGTCGAAATTTGAAACCAT
CTTCTTCACCATCTCTGGTCTCATCCATTACGCACCAGTGTTCAGAGACTATGTC
TTCCGGAGCATGCAGGAGTTCTACGAGGACAACGTGCTCTACATGGAGATCAGAG
CCACCCTGCTGCCGGTGTATGAGCTCAGTGGAGAGCACCATGACGAAGAGTGGTC
AGTGAAGACTTACCAGGAAGTAGCTCAGAAGTTTGTGGAAACTCACCCTGAGTTT
ATTGGAATCAAAATCATTTATGCAGATCACAGATCCAAAGATGTGGCTGTCATCG
CAGAATCCATCCGAATGGCCATGGGGCTCCGAATCAAGTTCCCCACGGTGGTGGC
AGGGTTTGACCTGGTGGGGCATGAGGACACTGGCCACTCCTTGCATGACTACAAG
GAAGCTCTGATGATCCCCGCCAAGGATGGCGTTAAGCTGCCTTACTTCTTCCACG
CCGGAGAAACAGACTGGCAGGGTACTTCCATAGACAGGAACATTCTGGATGCTCT
GATGCTGAACACTACCAGAATCGGCCATGGATTTGCTTTGAGCAAACACCCCGCA
GTCAGGACTTACTCCTGGAAAAAGGACATCCCCATAGAAGTCTGTCCCATCTCTA
ACCAGGTGCTGAAACTGGTGTCTGACTTGAGGAACCACCCTGTAGCCACTCTGAT
GGCCACTGGGCACCCCATGGTGATCAGCTCTGATGACCCAGCTATGTTTGGTGCC
AAAGGCTTGTCCTATGATTTCTATGAGGTCTTCATGGGCATTGGGGGGATGAAGG
CTGACCTGAGGACCCTCAAACAGCTGGCCATGAACTCTATCAAGTACAGTACCCT
GTTGGAGAGTGAGAAAAATACTTTCATGGAAATCTGGAAGAAGAGATGGGATAAG
TTCATAGCAGATGTGGCTACAAAGGGCGGACACCACCATCACCACCATGGAGGCT
AG
(Seq ID No. .)
MYRMQLLSCIALSLALVTNSQIDETRAHLLLKEKMMRLGGRLVLNTKEELANERL
MTLKIAEMKEAMRTLIFPPSMHFFQAKHLIERSQVFNILRMMPKGAALHLHDIGI
VTMDWLVRNVTYRPHCHICFTPRGIMQFRFAHPTPRPSEKCSKWILLEDYRKRVQ
NVTEFDDSLLRNFTLVTQHPEVIYTNQNVVWSKFETIFFTISGLIHYAPVFRDYV
FRSMQEFYEDNVLYMEIRATLLPVYELSGEHHDEEWSVKTYQEVAQKFVETHPEF
IGIKIIYADHRSKDVAVIAESIRMAMGLRIKFPTVVAGFDLVGHEDTGHSLHDYK
EALMIPAKDGVKLPYFFHAGETDWQGTSIDRNILDALMLNTTRIGHGFALSKHPA
VRTYSWKKDIPIEVCPISNQVLKLVSDLRNHPVATLMATGHPMVISSDDPAMFGA
KGLSYDFYEVFMGIGGMKADLRTLKQLAMNSIKYSTLLESEKNTFMEIWKKRWDK
FIADVATKGGHHHHHHGG
>PROS-HsADA2-(R222T, L224S, S265A, H301Q) Sequence|PROS SP
with HsADA2-Ph2 and 6xHis tag
(Seq ID No. .)
ATGAGGGTCCTGGGTGGGCGCTGCGGGGCGCTGCTGGCGTGTCTCCTCCTAGTGC
TTCCCGTGTCAGAGGCAGCCCAGATAGATGAAACACGGGCGCATCTGTTGTTGAA
AGAAAAGATGATGCGGCTGGGGGGGCGGCTGGTGCTGAACACCAAGGAGGAGCTG
GCCAATGAGAGGCTCATGACGCTCAAAATCGCTGAGATGAAGGAGGCCATGAGGA
CCCTGATATTCCCACCCAGCATGCACTTTTTCCAGGCCAAGCATCTCATTGAGAG
AAGTCAAGTGTTTAATATTCTAAGGATGATGCCAAAAGGGGCTGCCTTGCACCTC
CATGACATTGGCATCGTGACTATGGACTGGCTGGTGAGGAATGTCACCTACAGGC
CTCACTGCCACATCTGTTTCACCCCAAGGGGGATCATGCAGTTCAGATTTGCTCA
CCCAACTCCCCGTCCATCAGAAAAATGTTCCAAGTGGATTCTGCTGGAGGATTAT
CGGAAGCGGGTGCAGAACGTCACTGAGTTTGATGACAGCTTGCTGAGGAATTTCA
CTCTGGTGACCCAGCACCCGGAGGTGATTTACACAAACCAAAATGTTGTCTGGTC
GAAATTTGAAACCATCTTCTTCACCATCTCTGGTCTCATCCATTACGCACCAGTG
TTCAGAGACTATGTCTTCCGGAGCATGCAGGAGTTCTACGAGGACAACGTGCTCT
ACATGGAGATCAGAGCCACCCTGTCACCGGTGTATGAGCTCAGTGGAGAGCACCA
TGACGAAGAGTGGTCAGTGAAGACTTACCAGGAAGTAGCTCAGAAGTTTGTGGAA
ACTCACCCTGAGTTTATTGGAATCAAAATCATTTATGCAGATCACAGATCCAAAG
ATGTGGCTGTCATCGCAGAATCCATCCGAATGGCCATGGGGCTCCGAATCAAGTT
CCCCACGGTGGTGGCAGGGTTTGACCTGGTGGGGCAGGAGGACACTGGCCACTCC
TTGCATGACTACAAGGAAGCTCTGATGATCCCCGCCAAGGATGGCGTTAAGCTGC
CTTACTTCTTCCACGCCGGAGAAACAGACTGGCAGGGTACTTCCATAGACAGGAA
CATTCTGGATGCTCTGATGCTGAACACTACCAGAATCGGCCATGGATTTGCTTTG
AGCAAACACCCCGCAGTCAGGACTTACTCCTGGAAAAAGGACATCCCCATAGAAG
TCTGTCCCATCTCTAACCAGGTGCTGAAACTGGTGTCTGACTTGAGGAACCACCC
TGTAGCCACTCTGATGGCCACTGGGCACCCCATGGTGATCAGCTCTGATGACCCA
GCTATGTTTGGTGCCAAAGGCTTGTCCTATGATTTCTATGAGGTCTTCATGGGCA
TTGGGGGGATGAAGGCTGACCTGAGGACCCTCAAACAGCTGGCCATGAACTCTAT
CAAGTACAGTACCCTGTTGGAGAGTGAGAAAAATACTTTCATGGAAATCTGGAAG
AAGAGATGGGATAAGTTCATAGCAGATGTGGCTACAAAGGGCGGACACCACCATC
ACCACCATGGAGGCTAG
(Seq ID No. .)
MRVLGGRCGALLACLLLVLPVSEAAQIDETRAHLLLKEKMMRLGGRLVLNTKEEL
ANERLMTLKIAEMKEAMRTLIFPPSMHFFQAKHLIERSQVFNILRMMPKGAALHL
HDIGIVTMDWLVRNVTYRPHCHICFTPRGIMQFRFAHPTPRPSEKCSKWILLEDY
RKRVQNVTEFDDSLLRNFTLVTQHPEVIYTNQNVVWSKFETIFFTISGLIHYAPV
FRDYVFRSMQEFYEDNVLYMEIRATLSPVYELSGEHHDEEWSVKTYQEVAQKFVE
THPEFIGIKIIYADHRSKDVAVIAESIRMAMGLRIKFPTVVAGFDLVGQEDTGHS
LHDYKEALMIPAKDGVKLPYFFHAGETDWQGTSIDRNILDALMLNTTRIGHGFAL
SKHPAVRTYSWKKDIPIEVCPISNQVLKLVSDLRNHPVATLMATGHPMVISSDDP
AMFGAKGLSYDFYEVFMGIGGMKADLRTLKQLAMNSIKYSTLLESEKNTFMEIWK
KRWDKFIADVATKGGHHHHHHGG
(Seq ID No. .)
ALSIDETRAHLLLKEKMMRLGGRLVLNTKEELANERLMTLKIAEMKEAMRTLIFP
PSMHFFQAKHLIERSQVFNILRMMPKGAALHLHDIGIVTMDWLVRNVTYRPHCHI
CFTPRGIMQFRFAHPTPRPSEKCSKWILLEDYRKRVQNVTEFDDSLLRNFTLVTQ
HPEVIYTNQNVVWSKFETIFFTISGLIHYAPVFRDYVFRSMQEFYEDNVLYMEIR
ARLLPVYELSGEHHDEEWSVKTYQEVAQKFVETHPEFIGIKIIYSDHRSKDVAVI
AESIRMAMGLRIKFPTVVAGFDLVGHEDTGHSLHDYKEALMIPAKDGVKLPYFFH
AGETDWQGTSIDRNILDALMLNTTRIGHGFALSKHPAVRTYSWKKDIPIEVCPIS
NQVLKLVSDLRNHPVATLMATGHPMVISSDDPAMFGAKGLSYDFYEVFMGIGGMK
ADLRTLKQLAMNSIKYSTLLESEKNTFMEIWKKRWDKFIADVATK

Wildtype HsADA2 signal sequence MLVDGPSERPALCFLLLAVAMSFFGS (SEQ ID NO: 102)

### LIST OF EMBODIMENTS

The following is a non-exhaustive list of embodiments contemplated by the invention.

1. A nucleic acid molecule encoding human adenosine deaminase 2 (HsADA2), wherein the HsADA2 has been mutated to have increased catalytic activity relative to a wildtype HsADA2.

2. The nucleic acid molecule of item 1, wherein the HsADA2 has been mutated at one or more amino acid positions comprising an entry gate to a catalytic site of the enzyme.

3. The nucleic acid molecule of item 1 or 2, wherein the nucleic acid molecule comprises at least one mutation in positions V176-V197, M217-E228, 1262-A273, and/or F296-G305 of SEQ ID NO: 2.

4. The nucleic acid molecule of any of items 1-3, wherein the at least one mutation is in positions S179, E182, T183, S189, H193, A221, R222, L224, S265, D266, H267, S269, and/or H301 of SEQ ID NO: 2.

5. The nucleic acid molecule of any of items 1-4, wherein the at least one mutation alters the hydrophobicity of one or more of S179, E182, T183, S189, H193, A221, R222, L224, S265, D266, H267, S269, and/or H301 of SEQ ID NO: 2.

6. The nucleic acid molecule of any of items 1-5, wherein the at least one mutation is selected from the group consisting of: M, G, or V at position E182; S or T at position A221; Q, S, or T at position R222; S or P at position L224; N, T, or A at position S265; P or A at position D266; V or I at position H267; A, G, or M at position S269; and Q at position H301.

7. The nucleic acid molecule of any of items 1-6, wherein the at least one mutation comprises R222T, L224S, S265A, and/or H301Q.

8. The nucleic acid molecule of any of items 1-7, wherein the at least one mutation comprises R222T and/or S265A.

9. The nucleic acid molecule of any of items 1-8, further comprising at least one additional mutation in one or more of positions L87, 190, and/or 192.

10. The nucleic acid molecule of item 9, wherein the at least one additional mutation is selected from the group consisting of: V or F at L87; F at 190; and F at 192.

11. The nucleic acid molecule of any of items 1-10, wherein the nucleic acid molecule comprises a nucleotide sequence as set forth in SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 87, 89, 91, 93, 95, 97, and 99.

12. A vector or expression cassette comprising the nucleic acid molecule of any of items 1-11.

13. A cell comprising the vector or expression cassette of item 12.

14. The cell of item 13, wherein the cell is a mammalian cell comprising a healthy cell, a cancer cell, a tumor cell, and/or an immune cell, the immune cell preferably comprising a T cell, a CAR T cell, a natural killer (NK) cell, a B cell, and/or a neutrophil.

15. An amino acid sequence comprising at least one mutation at one or more amino acid positions of a human adenosine deaminase 2 (HsADA2) enzyme comprising an entry gate to a catalytic site of the HsADA2 enzyme, wherein the at least one mutation confers improved catalytic activity relative to a wildtype HsADA2.

16. The amino acid molecule of item 15, wherein the amino acid molecule comprises at least one mutation in positions V176-V197, M217-E228, 1262-A273, and/or F296-G305 of SEQ ID NO: 2.

17. The amino acid molecule of item 15 or 16, wherein the at least one mutation is in positions S179, E182, T183, S189, H193, A221, R222, L224, S265, D266, H267, S269, and/or H301 of SEQ ID NO: 2.

18. The amino acid molecule of any of items 15-17, wherein the at least one mutation alters the hydrophobicity of one or more of S179, E182, T183, S189, H193, A221, R222, L224, S265, D266, H267, S269, and/or H301 of SEQ ID NO: 2.

19. The amino acid molecule of any of items 15-18, wherein the at least one mutation is selected from the group consisting of: M, G, or V at position E182; S or T at position A221; Q, S, or T at position R222; S or P at position L224; N, T, or A at position

S265; P or A at position D266; V or I at position H267; A, G, or M at position S269; and Q at position H301.

20. The amino acid molecule of any of items 15-19, wherein the at least one mutation comprises R222T, L224S, S265A, and/or H301Q.

21. The amino acid molecule of any of items 15-20, wherein the at least one mutation comprises R222T and/or S265A.

22. The amino acid molecule of item 21, further comprising at least one additional mutation in one or more of positions L87, 190, and/or 192.

23. The amino acid molecule of any of items 15-22, wherein the at least one additional mutation is selected from the group consisting of: V or F at L87; F at 190; and F at 192.

24. The amino acid molecule of any of items 15-23, wherein the amino acid molecule comprises an amino acid sequence as set forth in SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 88, 90, 92, 94, 96, 98, and 100.

25. The amino acid molecule of any of items 15-24, wherein the amino acid molecule is operably fused to an Fc portion of an antibody, an scFv portion of an antibody, a collagen-like peptide, or a collagen-specific scFv portion of an antibody.

26. A cell comprising the amino acid molecule of any of items 15-25, wherein the amino acid molecule is expressed in and secreted from the cell.

27. The cell of item 26, wherein the cell is a mammalian cell comprising a healthy cell, a cancer cell, a tumor cell, and/or an immune cell, the immune cell preferably comprising a T cell, a CAR T cell, a natural killer (NK) cell, a B cell, and/or a neutrophil.

28. A composition comprising an amino acid sequence comprising at least one mutation at one or more amino acid positions of a human adenosine deaminase 2 (HsADA2) enzyme comprising an entry gate to a catalytic site of the HsADA2 enzyme, wherein the at least one mutation confers improved catalytic activity on the amino acid sequence relative to a wildtype HsADA2 enzyme.

29. The composition of item 28, wherein the amino acid molecule comprises at least one mutation in positions V176-V197, M217-E228, 1262-A273, and/or F296-G305 of SEQ ID NO: 2.

30. The composition of items 28 or 29, wherein the at least one mutation is in positions S179, E182, T183, S189, H193, A221, R222, L224, S265, D266, H267, S269, and/or H301 of SEQ ID NO: 2.

31. The composition of any of items 28-30, wherein the at least one mutation alters the hydrophobicity of one or more of S179, E182, T183, S189, H193, A221, R222, L224, S265, D266, H267, S269, and/or H301 of SEQ ID NO: 2.

32. The composition of any of items 28-31, wherein the at least one mutation is selected from the group consisting of: M, G, or V at position E182; S or T at position A221; Q, S, or T at position R222; S or P at position L224; N, T, or A at position S265; P or A at position D266; V or I at position H267; A, G, or M at position S269; and Q at position H301.

33. The composition of any of items 28-32, wherein the at least one mutation comprises R222T, L224S, S265A, and/or H301Q.

34. The composition of any of items 28-33, wherein the at least one mutation comprises R222T and/or S265A.

35. The composition of any of items 28-34, further comprising at least one additional mutation in one or more of positions L87, 190, and/or 192.

36. The composition of item 35, wherein the at least one additional mutation is selected from the group consisting of: V or F at L87; F at 190; and F at 192.

37. The composition of any of items 28-36, wherein the amino acid molecule comprises an amino acid sequence as set forth in SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 88, 90, 92, 94, 96, 98, and 100.

38. The composition of any of items 28-37, wherein the amino acid molecule is operably fused to an Fc portion of an antibody, an scFv portion of an antibody, a collagen-like peptide, or a collagen-specific scFv portion of an antibody.

39. The composition of any of items 28-38, wherein a therapeutically effective amount of the composition is administered to a subject in need thereof to treat a cancer or tumor in the subject, optionally in combination with one or more cancer immunotherapies, and wherein the composition is administered by any suitable route, such as intratumoral, peritumoral, subcutaneous, intradermal, intravenous, or intraperitoneal administration.

40. A composition comprising one or more cells, the one or more cells comprising an amino acid sequence comprising at least one mutation at one or more amino acid positions of a human adenosine deaminase 2 (HsADA2) enzyme comprising an entry gate to a catalytic site of the HsADA2 enzyme, wherein the one or more cells express and secrete the amino acid sequence, and wherein the at least one mutation confers improved catalytic activity on the amino acid sequence relative to a wildtype HsADA2 enzyme.

41. The composition of item 40, wherein the amino acid molecule comprises at least one mutation in positions V176-V197, M217-E228, 1262-A273, and/or F296-G305 of SEQ ID NO: 2.

42. The composition of item 40 or 41, wherein the at least one mutation is in positions S179, E182, T183, S189, H193, A221, R222, L224, S265, D266, H267, S269, and/or H301 of SEQ ID NO: 2.

43. The composition of any of items 40-42, wherein the at least one mutation alters the hydrophobicity of one or more of S179, E182, T183, S189, H193, A221, R222, L224, S265, D266, H267, S269, and/or H301 of SEQ ID NO: 2.

44. The composition of any of items 40-43, wherein the at least one mutation is selected from the group consisting of: M, G, or V at position E182; S or T at position A221; Q, S, or T at position R222; S or P at position L224; N, T, or A at position S265; P or A at position D266; V or I at position H267; A, G, or M at position S269; and Q at position H301.

45. The composition of any of items 40-44, wherein the at least one mutation comprises R222T, L224S, S265A, and/or H301Q.

46. The composition of any of items 40-45, wherein the at least one mutation comprises R222T and/or S265A.

47. The composition of any of items 40-46, further comprising at least one additional mutation in one or more of positions L87, 190, and/or 192.

48. The composition of item 47, wherein the at least one additional mutation is selected from the group consisting of: V or F at L87; F at 190; and F at 192.

49. The composition of any of items 40-48, wherein the amino acid molecule comprises an amino acid sequence as set forth in SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 88, 90, 92, 94, 96, 98, and 100.

50. The composition of any of items 40-49, wherein the one or more cells are mammalian cells such as an immune cell, a cancer cell, a tumor cell, a healthy non-immune cell, a T cell, a CAR T cell, a natural killer (NK) cell, a B cell, and/or a neutrophil.

51. The composition of any of items 40-50, wherein the amino acid molecule is operably fused to an Fc portion of an antibody, an scFv portion of an antibody, a collagen-like peptide, or a collagen-specific scFv portion of an antibody.

52. The composition of any of items 40-51 further comprising an excipient or carrier.

53. The composition of any of items 40-52, wherein a therapeutically effective amount of the composition is administered to a subject in need thereof to treat a cancer or tumor in the subject, optionally in combination with one or more cancer immunotherapies, and wherein the composition is administered by any suitable route, such as intratumoral, peritumoral, subcutaneous, intradermal, intravenous, or intraperitoneal administration.

54. A method of producing at least one cell configured to express a human adenosine deaminase 2 (HsADA2) polypeptide, the method comprising:
introducing a nucleic acid molecule encoding the HsADA2 polypeptide into the at least one cell via a viral vector, electroporation, or liposomal mediated introduction;
integrating the nucleic acid encoding the HsADA2 polypeptide into the genome of the at least one cell; and
culturing the at least one cell in vitro under conditions suitable for expression of the HsADA2 polypeptide and expansion of the at least one cell, wherein the HsADA2 polypeptide comprises at least one mutation at one or more amino acid positions comprising an entry gate to a catalytic site of the HsADA2 polypeptide, wherein the at least one mutation confers improved catalytic activity relative to a wildtype HsADA2 polypeptide, and wherein the nucleic acid molecule encoding the HsADA2 polypeptide comprises a strong promoter and a terminator operably connected to the nucleic acid.

55. The method of item 54, wherein the polypeptide comprises at least one mutation in positions V176-V197, M217-E228, 1262-A273, and/or F296-G305 of SEQ ID NO: 2.

56. The method of item 54 or 55, wherein the at least one mutation is in positions S179, E182, T183, S189, H193, A221, R222, L224, S265, D266, H267, S269, and/or H301 of SEQ ID NO: 2.

57. The method of any of items 54-56, wherein the at least one mutation alters the hydrophobicity of one or more of S179, E182, T183, S189, H193, A221, R222, L224, S265, D266, H267, S269, and/or H301 of SEQ ID NO: 2.

58. The method of any of items 54-57, wherein the at least one mutation is selected from the group consisting of: M, G, or V at position E182; S or T at position A221; Q, S, or T at position R222; S or P at position L224; N, T, or A at position S265; P or A at position D266; V or I at position H267; A, G, or M at position S269; and Q at position H301.

59. The method of any of items 54-58, wherein the at least one mutation comprises R222T, L224S, S265A, and/or H301Q.

60. The method of any of items 54-59, wherein the at least one mutation comprises R222T and/or S265A.

61. The method of any of items 54-60, wherein the polypeptide further comprises at least one additional mutation in one or more of positions L87, 190, and/or 192.

62. The method of item 61, wherein the at least one additional mutation is selected from the group consisting of: V or F at L87; F at 190; and F at 192.

63. The method of any of items 54-62, wherein the ADA2 polypeptide comprises an amino acid sequence as set forth in SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 88, 90, 92, 94, 96, 98, and 100.

64. The method of any of items 54-63, wherein the HsADA2 polypeptide is operably fused to an Fc portion of an antibody, an scFv portion of an antibody, a collagen-like peptide, or a collagen-specific scFv portion of an antibody.

65. The method of any of items 54-64, wherein the at least one cell comprises a mammalian cell such as an immune cell, a cancer cell, a tumor cell, a healthy non-immune cell, a T cell, a CAR T cell, a natural killer (NK) cell, a B cell, and/or a neutrophil.

66. A method of treating a cancer or tumor in a subject in need thereof, the method comprising:

administering to the subject a therapeutically effective amount of a composition comprising cells configured to express a human adenosine deaminase 2 (HsADA2) polypeptide, wherein the HsADA2 polypeptide comprises at least one mutation at one or more amino acid positions comprising an entry gate to a catalytic site of the HsADA2 polypeptide, and wherein the at least one mutation confers improved catalytic activity relative to a wildtype HsADA2 polypeptide.

67. The method of item 66, wherein the HsADA2 polypeptide comprises at least one mutation in positions V176-V197, M217-E228, 1262-A273, and/or F296-G305 of SEQ ID NO: 2.

68. The method of item 66 or 67, wherein the at least one mutation is in positions S179, E182, T183, S189, H193, A221, R222, L224, S265, D266, H267, S269, and/or H301 of SEQ ID NO: 2.

69. The method of any of items 66-68, wherein the at least one mutation alters the hydrophobicity of one or more of S179, E182, T183, S189, H193, A221, R222, L224, S265, D266, H267, S269, and/or H301 of SEQ ID NO: 2.

70. The method of any of items 66-69, wherein the at least one mutation is selected from the group consisting of: M, G, or V at position E182; S or T at position A221; Q, S, or T at position R222; S or P at position L224; N, T, or A at position S265; P or A at position D266; V or I at position H267; A, G, or M at position S269; and Q at position H301.

71. The method of any of items 66-70, wherein the at least one mutation comprises R222T, L224S, S265A, and/or H301Q.

72. The method of any of items 66-71, wherein the at least one mutation comprises R222T and/or S265A.

73. The method of any of items 66-72, wherein the polypeptide further comprises at least one additional mutation in one or more of positions L87, 190, and/or 192.

74. The method of item 73, wherein the at least one additional mutation is selected from the group consisting of: V or F at L87; F at 190; and F at 192.

75. The method of any of items 66-74, wherein the ADA2 polypeptide comprises an amino acid sequence as set forth in SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 88, 90, 92, 94, 96, 98, and 100.

76. The method of any of items 66-75, wherein the cells comprise mammalian cells such as an immune cell, a cancer cell, a tumor cell, a healthy non-immune cell, a T cell, a CAR T cell, a natural killer (NK) cell, a B cell, and/or a neutrophil.

77. The method of any of items 66-76, wherein the HsADA2 polypeptide is operably fused to an Fc portion of an antibody, an scFv portion of an antibody, a collagen-like peptide, or a collagen-specific scFv portion of an antibody.

78. The method of any of items 66-77, wherein the composition further comprises an excipient or a carrier.

79. The method of any of items 66-78, wherein the composition is administered in combination with one or more cancer immunotherapies.

80. The method of any of items 66-79, wherein the immune cells are further modified to express and secrete additional immunogenic proteins, or the immune cells are further modified to have inducible control over cell functions.

81. The method of any of items 66-80, wherein the composition is formulated for administration by any suitable route, such as intratumoral, peritumoral, intradermal, subcutaneous, intradermal, intravenous, or intraperitoneal administration.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

1. A nucleic acid molecule encoding human adenosine deaminase 2 (HsADA2), wherein the HsADA2 has been mutated to have increased catalytic activity relative to a wildtype HsADA2.

2. The nucleic acid molecule of claim 1, wherein the HsADA2 has been mutated at one or more amino acid positions comprising an entry gate to a catalytic site of the enzyme.

3. The nucleic acid molecule of claim 1, wherein the nucleic acid molecule comprises at least one mutation in position V176-V197, M217-E228, 1262-A273, and/or F296-G305 of SEQ ID NO: 2.

4. The nucleic acid molecule of claim 1, wherein at least one mutation is in position S179, E182, T183, S189, H193, A221, R222, L224, S265, D266, H267, S269, and/or H301 of SEQ ID NO: 2.

5. The nucleic acid molecule of claim 1, wherein at least one mutation alters the hydrophobicity of one or more of S179, E182, T183, S189, H193, A221, R222, L224, S265, D266, H267, S269, and/or H301 of SEQ ID NO: 2.

6. The nucleic acid molecule of claim 3, wherein at least one mutation is selected from the group consisting of:
M, G, or V at position E182;
S or T at position A221;
Q, S, or T at position R222;
S or P at position L224;
N, T, or A at position S265;
P or A at position D266;
V or I at position H267;
A, G, or M at position S269; and
Q at position H301.

7. The nucleic acid molecule of claim 6, wherein at least one mutation comprises R222T, L224S, S265A, and/or H301Q.

8. The nucleic acid molecule of claim 6, wherein at least one mutation comprises R222T and/or S265A.

9. The nucleic acid molecule of claim 3 further comprising at least one additional mutation in one or more of positions L87, 190, and/or 192.

10. The nucleic acid molecule of claim 9, wherein at least one of the additional mutations is selected from the group consisting of:
V or F at L87;
F at 190; and
F at 192.

11. The nucleic acid molecule of claim 1, wherein the nucleic acid molecule comprises a nucleotide sequence as set forth in SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 87, 89, 91, 93, 95, 97, and 99.

12. A vector or expression cassette comprising the nucleic acid molecule of claim 1.

13. A cell comprising the vector or expression cassette of claim 12.

14. The cell of claim 13, wherein the cell is a mammalian cell such as an immune cell, a cancer cell, a tumor cell, a healthy non-immune cell, a T cell, a CAR T cell, a natural killer (NK) cell, a B cell, and/or a neutrophil.

15. An amino acid sequence comprising at least one mutation at one or more amino acid positions of a human adenosine deaminase 2 (HsADA2) enzyme comprising an entry gate to a catalytic site of the HsADA2 enzyme, wherein the at least one mutation confers improved catalytic activity relative to a wildtype HsADA2.

16.-25. (canceled)

26. A cell comprising the amino acid sequence of claim 15.

27. (canceled)

28. A composition comprising the amino acid sequence of claim 15.

29.-37. (canceled)

38. The composition of claim 28, wherein the amino acid sequence is operably fused to an Fc portion of an antibody, an scFv portion of an antibody, a collagen-like peptide, or a collagen-specific scFv portion of an antibody.

39. The composition of claim 28, wherein a therapeutically effective amount of the composition is administered to a subject in need thereof to treat a cancer or tumor in the subject, optionally in combination with one or more cancer immunotherapies, and wherein the composition is formulated for administration by any suitable route, such as intratumoral, peritumoral, subcutaneous, intradermal, intravenous, or intraperitoneal administration.

40. A composition comprising one or more cells, the one or more cells comprising one or more cell of claim 13.

41.-53. (canceled)

54. A method of producing at least one cell configured to express a human adenosine deaminase 2 (HsADA2) polypeptide, the method comprising:
introducing a nucleic acid molecule encoding the HsADA2 polypeptide into the at least one cell via a viral vector, electroporation, or liposomal mediated introduction;
integrating the nucleic acid encoding the HsADA2 polypeptide into the genome of the at least one cell; and
culturing the at least one cell in vitro under conditions suitable for expression of the HsADA2 polypeptide and expansion of the at least one cell;
wherein the HsADA2 polypeptide comprises at least one mutation at one or more amino acid positions comprising an entry gate to a catalytic site of the HsADA2 polypeptide;
wherein the at least one mutation confers improved catalytic activity relative to a wildtype HsADA2 polypeptide; and
wherein the nucleic acid molecule encoding the HsADA2 polypeptide comprises a strong promoter and a terminator operably connected to the nucleic acid.

55.-65. (canceled)

66. A method of treating a cancer or tumor in a subject in need thereof, the method comprising:
administering to the subject a therapeutically effective amount of a composition comprising cells configured to express a human adenosine deaminase 2 (HsADA2) polypeptide;
wherein the HsADA2 polypeptide comprises at least one mutation at one or more amino acid positions comprising an entry gate to a catalytic site of the HsADA2 polypeptide, and wherein the at least one mutation confers improved catalytic activity relative to a wildtype HsADA2 polypeptide.

67.-81. (canceled)


SEQUENCE LISTING (203 sequences)

SEQ ID NO: 1 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaggctgctgccggtgtatgagctcagtggagagcaccatgacgaagagtggtcagtgaagacttaccaggaagtagctcagaagtttgtggaaactcaccctgagtttattggaatcaaaatcatttattcggatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 2 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 3 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagcccagctgctgccggtgtatgagctcagtggagagcaccatgacgaagagtggtcagtgaagacttaccaggaagtagctcagaagtttgtggaaactcaccctgagtttattggaatcaaaatcatttataatgatgtgagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 4 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRAQLLPVYELSGEHHDEEWSVKTYQEVAQKFVETHPEFIGIKIIYNDVRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 5 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagcctcgctgctgccggtgtatgagctcagtggagagcaccatgacgaagagtggtcagtgaagacttaccaggaagtagctcagaagtttgtggaaactcaccctgagtttattggaatcaaaatcatttattcggatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 6 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRASLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 7 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgctgccggtgtatgagctcagtggagagcaccatgacgaagagtggtcagtgaagacttaccaggaagtagctcagaagtttgtggaaactcaccctgagtttattggaatcaaaatcatttattcggatgtgagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 8 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLLPVYELSGEHHDEEWSVKTYQEVAQKFVETHPEFIGIKIIYSDVRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 9 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaggctgctgccggtgtatgagctcagtggagagcaccatgacgaagagtggtcagtgaagacttaccaggaagtagctcagaagtttgtggaaactcaccctgagtttattggaatcaaaatcatttataacgatgtgagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 10 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYNDVRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 11 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttatgaccatcttcttcaccatctctggtctcatccattacgcaccagtgttcagagactatgtcttccggagcatgcaggagttctacgaggacaacgtgctctacatggagatcagagcccagctgctgccggtgtatgagctcagtggagagcaccatgacgaagagtggtcagtgaagacttaccaggaagtagctcagaagtttgtggaaactcaccctgagtttattggaatcaaaatcatttatacggatgtgagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 12 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFMTIFFTISGLIHYAPVFRDYVFRSMQEFYEDNVLYMEIRAQLLPVYELSGEHHDEEWSVKTYQEVAQKFVETHPEFIGIKIIYTDVRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 13 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttggcaccatcttcttcaccatctctggtctcatccattacgcaccagtgttcagagactatgtcttccggagcatgcaggagttctacgaggacaacgtgctctacatggagatcagagccaccctgctgccggtgtatgagctcagtggagagcaccatgacgaagagtggtcagtgaagacttaccaggaagtagctcagaagtttgtggaaactcaccctgagtttattggaatcaaaatcatttataacgatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 14 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFGTIFFTISGLIHYAPVFRDYVFRSMQEFYEDNVLYMEIRATLLPVYELSGEHHDEEWSVKTYQEVAQKFVETHPEFIGIKIIYNDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 15 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgctgccggtgtatgagctcagtggagagcaccatgacgaagagtggtcagtgaagacttaccaggaagtagctcagaagtttgtggaaactcaccctgagtttattggaatcaaaatcatttataacgatgtgagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 16 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLLPVYELSGEHHDEEWSVKTYQEVAQKFVETHPEFIGIKIIYADVRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 17 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttggcaccatcttcttcaccatctctggtctcatccattacgcaccagtgttcagagactatgtcttccggagcatgcaggagttctacgaggacaacgtgctctacatggagatcagagcctcactgctgccggtgtatgagctcagtggagagcaccatgacgaagagtggtcagtgaagacttaccaggaagtagctcagaagtttgtggaaactcaccctgagtttattggaatcaaaatcatttataccgatataagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 18 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFGTIFFTISGLIHYAPVFRDYVFRSMQEFYEDNVLYMEIRASLLPVYELSGEHHDEEWSVKTYQEVAQKFVETHPEFIGIKIIYTDIRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 19 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgtaaccatcttcttcaccatctctggtctcatccattacgcaccagtgttcagagactatgtcttccggagcatgcaggagttctacgaggacaacgtgctctacatggagatcagagcctcactgctgccggtgtatgagctcagtggagagcaccatgacgaagagtggtcagtgaagacttaccaggaagtagctcagaagtttgtggaaactcaccctgagtttattggaatcaaaatcatttataccgatataagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 20 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFVTIFFTISGLIHYAPVFRDYVFRSMQEFYEDNVLYMEIRASLLPVYELSGEHHDEEWSVKTYQEVAQKFVETHPEFIGIKIIYTDIRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 21 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttggcaccatcttcttcaccatctctggtctcatccattacgcaccagtgttcagagactatgtcttccggagcatgcaggagttctacgaggacaacgtgctctacatggagatcagagcctcactgctgccggtgtatgagctcagtggagagcaccatgacgaagagtggtcagtgaagacttaccaggaagtagctcagaagtttgtggaaactcaccctgagtttattggaatcaaaatcatttatgcagatgtgagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 22 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFGTIFFTISGLIHYAPVFRDYVFRSMQEFYEDNVLYMEIRASLLPVYELSGEHHDEEWSVKTYQEVAQKFVETHPEFIGIKIIYADVRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 23 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgtgaccatcttcttcaccatctctggtctcatccattacgcaccagtgttcagagactatgtcttccggagcatgcaggagttctacgaggacaacgtgctctacatggagatcagagcccagctgctgccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 24 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFVTIFFTISGLIHYAPVFRDYVFRSMQEFYEDNVLYMEIRAQLLPVYELSGEHHDEEWSVKTYQEVAQKFVETHPEFIGIKIIYADHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 25 - DNA, 1566 bp, synthetic construct; identical to SEQ ID NO: 9
  source 1..1566: mol_type = other DNA

SEQ ID NO: 26 - protein, 521 aa, synthetic construct; identical to SEQ ID NO: 10

SEQ ID NO: 27 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagcccagctgctgccggtgtatgagctcagtggagagcaccatgacgaagagtggtcagtgaagacttaccaggaagtagctcagaagtttgtggaaactcaccctgagtttattggaatcaaaatcatttattcggatgtgagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 28 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRAQLLPVYELSGEHHDEEWSVKTYQEVAQKFVETHPEFIGIKIIYSDVRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 29 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaggctgctgccggtgtatgagctcagtggagagcaccatgacgaagagtggtcagtgaagacttaccaggaagtagctcagaagtttgtggaaactcaccctgagtttattggaatcaaaatcatttattcggatataagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 30 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDIRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 31 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagcccagctgctgccggtgtatgagctcagtggagagcaccatgacgaagagtggtcagtgaagacttaccaggaagtagctcagaagtttgtggaaactcaccctgagtttattggaatcaaaatcatttattcggatataagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 32 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRAQLLPVYELSGEHHDEEWSVKTYQEVAQKFVETHPEFIGIKIIYSDIRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 33 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgctgccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 34 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLLPVYELSGEHHDEEWSVKTYQEVAQKFVETHPEFIGIKIIYADHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 35 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgactttggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgtcaccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 36 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDFGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLSPVYELSGEHHDEEWSVKTYQEVAQKFVETHPEFIGIKIIYADHRSKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 37 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacgtgcatgactttggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgcccccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 38 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHVHDFGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLPPVYELSGEHHDEEWSVKTYQEVAQKFVETHPEFIGIKIIYADHRSKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 39 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgtcaccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 40 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLSPVYELSGEHHDEEWSVKTYQEVAQKFVETHPEFIGIKIIYADHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 41 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgactttggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgcccccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 42 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDFGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLPPVYELSGEHHDEEWSVKTYQEVAQKFVETHPEFIGIKIIYADHRSKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 43 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgtcaccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 44 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLSPVYELSGEHHDEEWSVKTYQEVAQKFVETHPEFIGIKIIYADHRSKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 45 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgctgccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 46 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLLPVYELSGEHHDEEWSVKTYQEVAQKFVETHPEFIGIKIIYADHRSKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 47 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagatcactgctgctgccggtgtatgagctcagtggagagcaccatgacgaagagtggtcagtgaagacttaccaggaagtagctcagaagtttgtggaaactcaccctgagtttattggaatcaaaatcatttatgcagatcacagagcaaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 48 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRSLLLPVYELSGEHHDEEWSVKTYQEVAQKFVETHPEFIGIKIIYADHRAKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 49 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagatcactcctgctgccggtgtatgagctcagtggagagcaccatgacgaagagtggtcagtgaagacttaccaggaagtagctcagaagtttgtggaaactcaccctgagtttattggaatcaaaatcatttatgcagatcacagaggcaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 50 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRSLLLPVYELSGEHHDEEWSVKTYQEVAQKFVETHPEFIGIKIIYADHRGKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 51 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagaaccctcctgctgccggtgtatgagctcagtggagagcaccatgacgaagagtggtcagtgaagacttaccaggaagtagctcagaagtttgtggaaactcaccctgagtttattggaatcaaaatcatttatgcaccccacagagcaaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 52 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRTLLLPVYELSGEHHDEEWSVKTYQEVAQKFVETHPEFIGIKIIYAPHRAKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 53 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagaaccatactgctgccggtgtatgagctcagtggagagcaccatgacgaagagtggtcagtgaagacttaccaggaagtagctcagaagtttgtggaaactcaccctgagtttattggaatcaaaatcatttatgcaccccacagagcaaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 54 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRTILLPVYELSGEHHDEEWSVKTYQEVAQKFVETHPEFIGIKIIYAPHRAKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 55 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagatcaatactgctgccggtgtatgagctcagtggagagcaccatgacgaagagtggtcagtgaagacttaccaggaagtagctcagaagtttgtggaaactcaccctgagtttattggaatcaaaatcatttatgcaccccacagagcaaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 56 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRSILLPVYELSGEHHDEEWSVKTYQEVAQKFVETHPEFIGIKIIYAPHRAKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 57 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagaaccctgctgctgccggtgtatgagctcagtggagagcaccatgacgaagagtggtcagtgaagacttaccaggaagtagctcagaagtttgtggaaactcaccctgagtttattggaatcaaaatcatttatgcaccccacagaatgaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 58 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRTLLLPVYELSGEHHDEEWSVKTYQEVAQKFVETHPEFIGIKIIYAPHRMKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 59 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagatcactgctgctgccggtgtatgagctcagtggagagcaccatgacgaagagtggtcagtgaagacttaccaggaagtagctcagaagtttgtggaaactcaccctgagtttattggaatcaaaatcatttatgcaccccacagaatgaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 60 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRSLLLPVYELSGEHHDEEWSVKTYQEVAQKFVETHPEFIGIKIIYAPHRMKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 61 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagatcactgctgctgccggtgtatgagctcagtggagagcaccatgacgaagagtggtcagtgaagacttaccaggaagtagctcagaagtttgtggaaactcaccctgagtttattggaatcaaaatcatttatgcaccccacagaggcaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 62 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRSLLLPVYELSGEHHDEEWSVKTYQEVAQKFVETHPEFIGIKIIYAPHRGKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 63 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagatcaatactgctgccggtgtatgagctcagtggagagcaccatgacgaagagtggtcagtgaagacttaccaggaagtagctcagaagtttgtggaaactcaccctgagtttattggaatcaaaatcatttatgcaccccacagaggcaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 64 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRSILLPVYELSGEHHDEEWSVKTYQEVAQKFVETHPEFIGIKIIYAPHRGKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 65 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgtcaccggtgtatgagctcagtggagagcaccatgacgaagagtggtcagtgaagacttaccaggaagtagctcagaagtttgtggaaactcaccctgagtttattggaatcaaaatcatttatgcaccccacagatccaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 66 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLSPVYELSGEHHDEEWSVKTYQEVAQKFVETHPEFIGIKIIYAPHRSKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 67 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccatgctgctgccggtgtatgagctcagtggagagcaccatgacgaagagtggtcagtgaagacttaccaggaagtagctcagaagtttgtggaaactcaccctgagtttattggaatcaaaatcatttatgcaccccacagaatgaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 68 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRAMLLPVYELSGEHHDEEWSVKTYQEVAQKFVETHPEFIGIKIIYAPHRMKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 69 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagaaccaccctgctgccggtgtatgagctcagtggagagcaccatgacgaagagtggtcagtgaagacttaccaggaagtagctcagaagtttgtggaaactcaccctgagtttattggaatcaaaatcatttatgcaccccacagatccaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 70 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRTTLLPVYELSGEHHDEEWSVKTYQEVAQKFVETHPEFIGIKIIYAPHRSKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 71 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagatcactgctgtcaccggtgtatgagctcagtggagagcaccatgacgaagagtggtcagtgaagacttaccaggaagtagctcagaagtttgtggaaactcaccctgagtttattggaatcaaaatcatttatgcaccccacagaatgaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 72 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRSLLSPVYELSGEHHDEEWSVKTYQEVAQKFVETHPEFIGIKIIYAPHRMKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 73 - DNA, 1566 bp, synthetic construct
  source 1..1566: mol_type = other DNA
atgttggtggatggcccatctgagcggccagccctgtgcttcttgctgttggctgtggcaatgtctttcttcggctcagctctatccatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagaaccctgctgtcaccggtgtatgagctcagtggagagcaccatgacgaagagtggtcagtgaagacttaccaggaagtagctcagaagtttgtggaaactcaccctgagtttattggaatcaaaatcatttatgcaccccacagaatgaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 74 - protein, 521 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRTLLSPVYELSGEHHDEEWSVKTYQEVAQKFVETHPEFIGIKIIYAPHRMKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 75 - DNA, 63 bp, synthetic construct
  source 1..63: mol_type = other DNA
atgggcagcgccgctctcctgctctgggtgctgctgctgtgggtgcccagctccagggcccag

SEQ ID NO: 76 - protein, 21 aa, synthetic construct
MGSAALLLWVLLLWVPSSRAQ

SEQ ID NO: 77 - DNA, 55 bp, synthetic construct
  source 1..55: mol_type = other DNA
atgcaaccaatcctgcttctgctggccttcctcctgctgcccagggcagatgcag

SEQ ID NO: 78 - protein, 19 aa, synthetic construct
MQPILLLLAFLLLPRADAQ

SEQ ID NO: 79 - DNA, 66 bp, synthetic construct
  source 1..66: mol_type = other DNA
atgagcgcggtattactgctggccctcctggggttcatcctcccactgccaggagtgcaggcgcag

SEQ ID NO: 80 - protein, 22 aa, synthetic construct
MSAVLLLALLGFILPLPGVQAQ

SEQ ID NO: 81 - DNA, 57 bp, synthetic construct
  source 1..57: mol_type = other DNA
atgaagtgggtaacctttatttcccttctttttctctttagctcggcttattcccag

SEQ ID NO: 82 - protein, 19 aa, synthetic construct
MKWVTFISLLFLFSSAYSQ

SEQ ID NO: 83 - DNA, 63 bp, synthetic construct
  source 1..63: mol_type = other DNA
atgtacaggatgcaactcctgtcttgcattgcactaagtcttgcacttgtcacaaacagtcag

SEQ ID NO: 84 - protein, 21 aa, synthetic construct
MYRMQLLSCIALSLALVTNSQ

SEQ ID NO: 85 - DNA, 78 bp, synthetic construct
  source 1..78: mol_type = other DNA
atgagggtcctgggtgggcgctgcggggcgctgctggcgtgtctcctcctagtgcttcccgtgtcagaggcagcccag

SEQ ID NO: 86 - protein, 26 aa, synthetic construct
MRVLGGRCGALLACLLLVLPVSEAAQ

SEQ ID NO: 87 - DNA, 1557 bp, synthetic construct
  source 1..1557: mol_type = other DNA
atgagggtcctgggtgggcgctgcggggcgctgctggcgtgtctcctcctagtgcttcccgtgtcagaggcagcccagatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgctgccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 88 - protein, 518 aa, synthetic construct
MRVLGGRCGALLACLLLVLPVSEAAQIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLLPVYELSGEHHDEEWSVKTYQEVAQKFVETHPEFIGIKIIYADHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 89 - DNA, 1542 bp, synthetic construct
  source 1..1542: mol_type = other DNA
atgggcagcgccgctctcctgctctgggtgctgctgctgtgggtgcccagctccagggcccagatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgctgccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 90 - protein, 513 aa, synthetic construct
MGSAALLLWVLLLWVPSSRAQIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLLPVYELSGEHHDEEWSVKTYQEVAQKFVETHPEFIGIKIIYADHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 91 - DNA, 1534 bp, synthetic construct
  source 1..1534: mol_type = other DNA
atgcaaccaatcctgcttctgctggccttcctcctgctgcccagggcagatgcagatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgctgccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 92 - protein, 511 aa, synthetic construct
MQPILLLLAFLLLPRADAQIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLLPVYELSGEHHDEEWSVKTYQEVAQKFVETHPEFIGIKIIYADHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 93 - DNA, 1545 bp, synthetic construct
  source 1..1545: mol_type = other DNA
atgagcgcggtattactgctggccctcctggggttcatcctcccactgccaggagtgcaggcgcagatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgctgccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 94 - protein, 514 aa, synthetic construct
MSAVLLLALLGFILPLPGVQAQIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLLPVYELSGEHHDEEWSVKTYQEVAQKFVETHPEFIGIKIIYADHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 95 - DNA, 1536 bp, synthetic construct
  source 1..1536: mol_type = other DNA
atgaagtgggtaacctttatttcccttctttttctctttagctcggcttattcccagatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgctgccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 96 - protein, 511 aa, synthetic construct
MKWVTFISLLFLFSSAYSQIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLLPVYELSGEHHDEEWSVKTYQEVAQKFVETHPEFIGIKIIYADHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 97 - DNA, 1542 bp, synthetic construct
  source 1..1542: mol_type = other DNA
atgtacaggatgcaactcctgtcttgcattgcactaagtcttgcacttgtcacaaacagtcagatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgctgccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcatgaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 98 - protein, 513 aa, synthetic construct
MYRMQLLSCIALSLALVTNSQIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLLPVYELSGEHHDEEWSVKTYQEVAQKFVETHPEFIGIKIIYADHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 99 - DNA, 1557 bp, synthetic construct
  source 1..1557: mol_type = other DNA
atgagggtcctgggtgggcgctgcggggcgctgctggcgtgtctcctcctagtgcttcccgtgtcagaggcagcccagatagatgaaacacgggcgcatctgttgttgaaagaaaagatgatgcggctgggggggcggctggtgctgaacaccaaggaggagctggccaatgagaggctcatgacgctcaaaatcgctgagatgaaggaggccatgaggaccctgatattcccacccagcatgcactttttccaggccaagcatctcattgagagaagtcaagtgtttaatattctaaggatgatgccaaaaggggctgccttgcacctccatgacattggcatcgtgactatggactggctggtgaggaatgtcacctacaggcctcactgccacatctgtttcaccccaagggggatcatgcagttcagatttgctcacccaactccccgtccatcagaaaaatgttccaagtggattctgctggaggattatcggaagcgggtgcagaacgtcactgagtttgatgacagcttgctgaggaatttcactctggtgacccagcacccggaggtgatttacacaaaccaaaatgttgtctggtcgaaatttgaaaccatcttcttcaccatctctggtctcatccattacgcaccagtgttcagagactatgtcttccggagcatgcaggagttctacgaggacaacgtgctctacatggagatcagagccaccctgtcaccggtgtatgagctcagtggagagcaccatgacgaagagtggtcagtgaagacttaccaggaagtagctcagaagtttgtggaaactcaccctgagtttattggaatcaaaatcatttatgcagatcacagatccaaagatgtggctgtcatcgcagaatccatccgaatggccatggggctccgaatcaagttccccacggtggtggcagggtttgacctggtggggcaggaggacactggccactccttgcatgactacaaggaagctctgatgatccccgccaaggatggcgttaagctgccttacttcttccacgccggagaaacagactggcagggtacttccatagacaggaacattctggatgctctgatgctgaacactaccagaatcggccatggatttgctttgagcaaacaccccgcagtcaggacttactcctggaaaaaggacatccccatagaagtctgtcccatctctaaccaggtgctgaaactggtgtctgacttgaggaaccaccctgtagccactctgatggccactgggcaccccatggtgatcagctctgatgacccagctatgtttggtgccaaaggcttgtcctatgatttctatgaggtcttcatgggcattggggggatgaaggctgacctgaggaccctcaaacagctggccatgaactctatcaagtacagtaccctgttggagagtgagaaaaatactttcatggaaatctggaagaagagatgggataagttcatagcagatgtggctacaaagggcggacaccaccatcaccaccatggaggctag

SEQ ID NO: 100 - protein, 518 aa, synthetic construct
MRVLGGRCGALLACLLLVLPVSEAAQIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRATLSPVYELSGEHHDEEWSVKTYQEVAQKFVETHPEFIGIKIIYADHRSKDVAVIAESIRMAMGLRIKFPTVVAGFDLVGQEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGGHHHHHHGG

SEQ ID NO: 101 - protein, 485 aa, synthetic construct
ALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 102 - protein, 26 aa, synthetic construct
MLVDGPSERPALCFLLLAVAMSFFGS

SEQ ID NO: 103 - DNA, 40 bp, synthetic construct
  source 1..40: mol_type = other DNA
aaatgccaccatgtgagacgagttcgagatcgagggcgag

SEQ ID NO: 104 - DNA, 69 bp, synthetic construct
  source 1..69: mol_type = other DNA
gtccccgcatgttagaagacttcccctgccctctccagagcctgagacggcttcttggccttgtaggtg

SEQ ID NO: 105 - DNA, 61 bp, synthetic construct
  source 1..61: mol_type = other DNA
gtcttctaacatgcggggacgtggaggaaaatcccggcccaatggtgagcaagggcgagga

SEQ ID NO: 106 - DNA, 32 bp, synthetic construct
  source 1..32: mol_type = other DNA
tgaagctgttacttgtacagctcgtccatgcc

SEQ ID NO: 107 - DNA, 35 bp, synthetic construct
  source 1..35: mol_type = other DNA
ctgtacaagtaacagcttcagggatcgtacttcca

SEQ ID NO: 108 - DNA, 19 bp, synthetic construct
  source 1..19: mol_type = other DNA
gcgtgggtcccgcggtatc

SEQ ID NO: 109 - DNA, 19 bp, synthetic construct
  source 1..19: mol_type = other DNA
gataccgcgggacccacgc

SEQ ID NO: 110 - DNA, 45 bp, synthetic construct
  source 1..45: mol_type = other DNA
aactcgtctcacatggtggcatttcgataagccagtaagcagtgg

SEQ ID NO: 111 - DNA, 37 bp, synthetic construct
  source 1..37: mol_type = other DNA
gatcgatccgtctcacatggcccagacgcccgccttc

SEQ ID NO: 112 - DNA, 39 bp, synthetic construct
  source 1..39: mol_type = other DNA
gatcgatccgtctcaagccgcctccatggtggtgatggt

SEQ ID NO: 113 - DNA, 80 bp, synthetic construct
  source 1..80: mol_type = other DNA
actggcttatcgaaatgccaccatggagacgccccgccgacatccccgactacttgcgtctcgcccagacgcccgccttc

SEQ ID NO: 114 - DNA, 20 bp, synthetic construct
  source 1..20: mol_type = other DNA
acttcccctgccctctccag

SEQ ID NO: 115 - DNA, 90 bp, synthetic construct
  source 1..90: mol_type = other DNA
ccatgttggtggatggcccatctgagcggccagccctgtgcttcttgctgttggctgtggcaatgtctttcttcggctcagctctatccg

SEQ ID NO: 116 - DNA, 90 bp, synthetic construct
  source 1..90: mol_type = other DNA
tgggcggatagagctgagccgaagaaagacattgccacagccaacagcaagaagcacagggctggccgctcagatgggccatccaccaac

SEQ ID NO: 117 - DNA, 57 bp, synthetic construct
  source 1..57: mol_type = other DNA
ccatgaagtgggtaacctttatttcccttctttttctctttagctcggcttattccg

SEQ ID NO: 118 - DNA, 57 bp, synthetic construct
  source 1..57: mol_type = other DNA
tgggcggaataagccgagctaaagagaaaaagaagggaaataaaggttacccacttc

SEQ ID NO: 119 - DNA, 63 bp, synthetic construct
  source 1..63: mol_type = other DNA
ccatgggcagcgccgctctcctgctctgggtgctgctgctgtgggtgcccagctccagggccg

SEQ ID NO: 120 - DNA, 63 bp, synthetic construct
  source 1..63: mol_type = other DNA
tgggcggccctggagctgggcacccacagcagcagcacccagagcaggagagcggcgctgccc

SEQ ID NO: 121 - DNA, 63 bp, synthetic construct
  source 1..63: mol_type = other DNA
ccatgtacaggatgcaactcctgtcttgcattgcactaagtcttgcacttgtcacaaacagtg

SEQ ID NO: 122 - DNA, 63 bp, synthetic construct
  source 1..63: mol_type = other DNA
tgggcactgtttgtgacaagtgcaagacttagtgcaatgcaagacaggagttgcatcctgtac

SEQ ID NO: 123 - DNA, 69 bp, synthetic construct
  source 1..69: mol_type = other DNA
ccatggcccgcgcagccccgctgctcgccgcgttgaccgcgctcctcgccgccgccgctgctggcggag

SEQ ID NO: 124 - DNA, 69 bp, synthetic construct
  source 1..69: mol_type = other DNA
tgggctccgccagcagcggcggcggcgaggagcgcggtcaacgcggcgagcagcggggctgcgcgggcc

SEQ ID NO: 125 - DNA, 75 bp, synthetic construct
  source 1..75: mol_type = other DNA
ccatgaagttacagtgtgtttccctttggctcctgggtacaatactgatattgtgctcagtagacaaccacggtg

SEQ ID NO: 126 - DNA, 75 bp, synthetic construct
  source 1..75: mol_type = other DNA
tgggcaccgtggttgtctactgagcacaatatcagtattgtacccaggagccaaagggaaacacactgtaacttc

SEQ ID NO: 127 - DNA, 66 bp, synthetic construct
  source 1..66: mol_type = other DNA
ccatgagcgcggtattactgctggccctcctggggttcatcctcccactgccaggagtgcaggcgg

SEQ ID NO: 128 - DNA, 66 bp, synthetic construct
  source 1..66: mol_type = other DNA
tgggccgcctgcactcctggcagtgggaggatgaaccccaggagggccagcagtaataccgcgctc

SEQ ID NO: 129 - DNA, 75 bp, synthetic construct
  source 1..75: mol_type = other DNA
ccatgagggtcctgggtgggcgctgcggggcgctgctggcgtgtctcctcctagtgcttcccgtgtcagaggcag

SEQ ID NO: 130 - DNA, 75 bp, synthetic construct
  source 1..75: mol_type = other DNA
tgggctgcctctgacacgggaagcactaggaggagacacgccagcagcgccccgcagcgcccacccaggaccctc

SEQ ID NO: 131 - DNA, 87 bp, synthetic construct
  source 1..87: mol_type = other DNA
ccatggctctgcagaggacccattcattgcttctgcttttgctgctgaccctgctggggctggggctggtccagccctcctatggcg

SEQ ID NO: 132 - DNA, 87 bp, synthetic construct
  source 1..87: mol_type = other DNA
tgggcgccataggagggctggaccagccccagccccagcagggtcagcagcaaaagcagaagcaatgaatgggtcctctgcagagcc

SEQ ID NO: 133 - DNA, 75 bp, synthetic construct
  source 1..75: mol_type = other DNA
ccatggatcgcctacagactgcactcctggttgtcctcgtcctccttgctgtggcgcttcaagcaactgaggcag

SEQ ID NO: 134 - DNA, 75 bp, synthetic construct
  source 1..75: mol_type = other DNA
tgggctgcctcagttgcttgaagcgccacagcaaggaggacgaggacaaccaggagtgcagtctgtaggcgatcc

SEQ ID NO: 135 - DNA, 57 bp, synthetic construct
  source 1..57: mol_type = other DNA
ccatgcaaccaatcctgcttctgctggccttcctcctgctgcccagggcagatgcag

SEQ ID NO: 136 - DNA, 57 bp, synthetic construct
  source 1..57: mol_type = other DNA
tgggctgcatctgccctgggcagcaggaggaaggccagcagaagcaggattggttgc

SEQ ID NO: 137 - DNA, 60 bp, synthetic construct
  source 1..60: mol_type = other DNA
ccatgaaaacgctccttcttttgctgctggtgctcctggagctgggagaggcccaaggag

SEQ ID NO: 138 - DNA, 60 bp, synthetic construct
  source 1..60: mol_type = other DNA
tgggctccttgggcctctcccagctccaggagcaccagcagcaaaagaaggagcgttttc

SEQ ID NO: 139 - DNA, 57 bp, synthetic construct
  source 1..57: mol_type = other DNA
ccatgcggcttctggtcctgctatggggttgcctgctgctcccaggttatgaagccg

SEQ ID NO: 140 - DNA, 57 bp, synthetic construct
  source 1..57: mol_type = other DNA
tgggcggcttcataacctgggagcagcaggcaaccccatagcaggaccagaagccgc

SEQ ID NO: 141 - DNA, 39 bp, synthetic construct
  source 1..39: mol_type = other DNA
gatcgatccgtctcacatgacgtcgtccgtctcacatgt

SEQ ID NO: 142 - DNA, 38 bp, synthetic construct
  source 1..38: mol_type = other DNA
gatcgatccgtctcaagccagcatgcacgtctcaagcc

SEQ ID NO: 143 - DNA, 45 bp, synthetic construct
  source 1..45: mol_type = other DNA
agctgacaagcgtctcaccatggccctgtccttttctttactgat

SEQ ID NO: 144 - DNA, 80 bp, synthetic construct
  source 1..80: mol_type = other DNA
ttcatcgactcgtctcctgggcgctcccgccacctccactcccgccgccaccttccttcctccttaatctttcttgaaaa

SEQ ID NO: 145 - DNA, 34 bp, synthetic construct
  source 1..34: mol_type = other DNA
agctgacaagcgtctcaggaggtggaggaagcgg

SEQ ID NO: 146 - DNA, 43 bp, synthetic construct
  source 1..43: mol_type = other DNA
ttcatcgactcgtctcctgggcgccctggaagtagaggttttc

SEQ ID NO: 147 - DNA, 35 bp, synthetic construct
  source 1..35: mol_type = other DNA
agctgacaagcgtctcaggtggtggtggtagcggt

SEQ ID NO: 148 - DNA, 38 bp, synthetic construct
  source 1..38: mol_type = other DNA
ttcatcgactcgtctcctgggcactcccgcccccaccg

SEQ ID NO: 149 - DNA, 71 bp, synthetic construct
  source 1..71: mol_type = other DNA
ccatgcggcctacttgggcctggtggctgttcctggtgctgctgctagctctgtgggcacccgctagaggg

SEQ ID NO: 150 - DNA, 71 bp, synthetic construct
  source 1..71: mol_type = other DNA
ctccccctctagcgggtgcccacagagctagcagcagcaccaggaacagccaccaggcccaagtaggccgc

SEQ ID NO: 151 - DNA, 71 bp, synthetic construct
  source 1..71: mol_type = other DNA
caccccctctagcgggtgcccacagagctagcagcagcaccaggaacagccaccaggcccaagtaggccgc

SEQ ID NO: 152 - DNA, 39 bp, synthetic construct
  source 1..39: mol_type = other DNA
agctagcaagcgtctcacctcggaggaggtggctctggc

SEQ ID NO: 153 - DNA, 41 bp, synthetic construct
  source 1..41: mol_type = other DNA
agctagcaagcgtctcaagccgcctccatggtggtgatggt

SEQ ID NO: 154 - DNA, 70 bp, synthetic construct
  source 1..70: mol_type = other DNA
catgcggcctacttgggcctggtggctgttcctggtgctgctgctagctctgtgggcacccgctagaggg

SEQ ID NO: 155 - DNA, 70 bp, synthetic construct
  source 1..70: mol_type = other DNA
gggcccctctagcgggtgcccacagagctagcagcagcaccaggaacagccaccaggcccaagtaggccg

SEQ ID NO: 156 - DNA, 34 bp, synthetic construct
  source 1..34: mol_type = other DNA
agctagcaagcgtctcagcccagacgcccgcctt

SEQ ID NO: 157 - DNA, 37 bp, synthetic construct
  source 1..37: mol_type = other DNA
agctagcaagcgtctcagaggttctgccctgcagagg

SEQ ID NO: 158 - DNA, 38 bp, synthetic construct
  source 1..38: mol_type = other DNA
agctagcaagcgtctcacatggcccagacgcccgcctt

SEQ ID NO: 159 - DNA, 36 bp, synthetic construct
  source 1..36: mol_type = other DNA
agctagcaagcgtctcaactcacacatccccaccga

SEQ ID NO: 160 - DNA, 37 bp, synthetic construct
  source 1..37: mol_type = other DNA
agctagcaagcgtctcatttacccggagacagggaga

SEQ ID NO: 161 - DNA, 69 bp, synthetic construct
  source 1..69: mol_type = other DNA
agctagcaagcgtctcataaaggaggaggtggctctggcggtggaggttccgcccagacgcccgccttc

SEQ ID NO: 162 - DNA, 42 bp, synthetic construct
  source 1..42: mol_type = other DNA
agctagcaagcgtctcaagccgcctccatggtggtgatggtg

SEQ ID NO: 163 - DNA, 70 bp, synthetic construct
  source 1..70: mol_type = other DNA
gagtccctctagcgggtgcccacagagctagcagcagcaccaggaacagccaccaggcccaagtaggccg

SEQ ID NO: 164 - DNA, 39 bp, synthetic construct
  source 1..39: mol_type = other DNA
agctagcaagcgtctcacatgactcacacatccccaccg

SEQ ID NO: 165 - DNA, 72 bp, synthetic construct
  source 1..72: mol_type = other DNA
tgggcccctctagcgggtgcccacagagctagcagcagcaccaggaacagccaccaggcccaagtaggccgc

SEQ ID NO: 166 - DNA, 39 bp, synthetic construct
  source 1..39: mol_type = other DNA
agctgacaagcgtctcaccatgggaggtggaggaagcgg

SEQ ID NO: 167 - DNA, 40 bp, synthetic construct
  source 1..40: mol_type = other DNA
agctgacaagcgtctcaccatgggtggtggtggtagcggt

SEQ ID NO: 168 - DNA, 36 bp, synthetic construct
  source 1..36: mol_type = other DNA
gctactgacgtctcacatggcccagacgcccgcctt

SEQ ID NO: 169 - DNA, 39 bp, synthetic construct
  source 1..39: mol_type = other DNA
gctactgacgtctcaagccgcctccatggtggtgatggt

SEQ ID NO: 170 - DNA, 39 bp, synthetic construct
  source 1..39: mol_type = other DNA
gctactgacgtctcacatgactcacacatccccaccgag

SEQ ID NO: 171 - DNA, 46 bp, synthetic construct
  source 1..46: mol_type = other DNA
gcttactggcttatcgaaatgccaccatgttggtggatggcccatc

SEQ ID NO: 172 - DNA, 23 bp, synthetic construct
  source 1..23: mol_type = other DNA
ccggcagcagctgggctctgatc

SEQ ID NO: 173 - DNA, 23 bp, synthetic construct
  source 1..23: mol_type = other DNA
gatcagagcccagctgctgccgg

SEQ ID NO: 174 - DNA, 33 bp, synthetic construct
  source 1..33: mol_type = other DNA
tttggatctgtgatcgttataaatgattttgat

SEQ ID NO: 175 - DNA, 33 bp, synthetic construct
  source 1..33: mol_type = other DNA
atcaaaatcatttataacgatcacagatccaaa

SEQ ID NO: 176 - DNA, 47 bp, synthetic construct
  source 1..47: mol_type = other DNA
agaagacttcccctgccctctccagagccgcctccatggtggtgatg

SEQ ID NO: 177 - DNA, 53 bp, synthetic construct
  source 1..53: mol_type = other DNA
tccgattgactgagtcgcccggatcccgccaccatgttggtggatggcccatc

SEQ ID NO: 178 - DNA, 48 bp, synthetic construct
  source 1..48: mol_type = other DNA
aagttattaggtccctcgacgaattcttacttgtacagctcgtccatg

SEQ ID NO: 179 - DNA, 35 bp, synthetic construct
  source 1..35: mol_type = other DNA
tggtgaagaagatggttgtaaatttcgaccagaca

SEQ ID NO: 180 - DNA, 35 bp, synthetic construct
  source 1..35: mol_type = other DNA
tgtctggtcgaaatttacaaccatcttcttcacca

SEQ ID NO: 181 - DNA, 67 bp, synthetic construct
  source 1..67: mol_type = other DNA
gtcttctaacatgcggggacgtggaggaaaatcccggcccaatgggagtcaaagttctgtttgccct

SEQ ID NO: 182 - DNA, 39 bp, synthetic construct
  source 1..39: mol_type = other DNA
gtacgatccctgaagctgttagtcaccaccggccccctt

SEQ ID NO: 183 - DNA, 24 bp, synthetic construct
  source 1..24: mol_type = other DNA
cagcttcagggatcgtacttccaa

SEQ ID NO: 184 - DNA, 35 bp, synthetic construct
  source 1..35: mol_type = other DNA
cgtactgacgtctcacatggtgagcaagggcgagg

SEQ ID NO: 185 - DNA, 40 bp, synthetic construct
  source 1..40: mol_type = other DNA
gatccatgcgtctcaagcccttgtacagctcgtccatgcc

SEQ ID NO: 186 - DNA, 38 bp, synthetic construct
  source 1..38: mol_type = other DNA
accatcaccaccatggaggcggctctggagagggcagg

SEQ ID NO: 187 - DNA, 43 bp, synthetic construct
  source 1..43: mol_type = other DNA
atgttgtctggtcgaaatttgdaaccatcttcttcaccatctc

SEQ ID NO: 188 - DNA, 43 bp, synthetic construct
  source 1..43: mol_type = other DNA
atgttgtctggtcgaaatttatgaccatcttcttcaccatctc

SEQ ID NO: 189 - DNA, 43 bp, synthetic construct
  source 1..43: mol_type = other DNA
tctacatggagatcagagcccractgctgccggtgtatgagct

SEQ ID NO: 190 - DNA, 43 bp, synthetic construct
  source 1..43: mol_type = other DNA
tctacatggagatcagagccascctgctgccggtgtatgagct

SEQ ID NO: 191 - DNA, 64 bp, synthetic construct
  source 1..64: mol_type = other DNA
ccctgagtttattggaatcaaaatcatttatrcagatsacagatccaaagatgtggctgtcatc

SEQ ID NO: 192 - DNA, 64 bp, synthetic construct
  source 1..64: mol_type = other DNA
ccctgagtttattggaatcaaaatcatttatrcagatrtaagatccaaagatgtggctgtcatc

SEQ ID NO: 193 - DNA, 64 bp, synthetic construct
  source 1..64: mol_type = other DNA
ccctgagtttattggaatcaaaatcatttatarcgatrtaagatccaaagatgtggctgtcatc

SEQ ID NO: 194 - DNA, 64 bp, synthetic construct
  source 1..64: mol_type = other DNA
ccctgagtttattggaatcaaaatcatttatarcgatsacagatccaaagatgtggctgtcatc

SEQ ID NO: 195 - DNA, 20 bp, synthetic construct
  source 1..20: mol_type = other DNA
aaatttcgaccagacaacat

SEQ ID NO: 196 - DNA, 20 bp, synthetic construct
  source 1..20: mol_type = other DNA
ggctctgatctccatgtaga

SEQ ID NO: 197 - DNA, 31 bp, synthetic construct
  source 1..31: mol_type = other DNA
ataaatgattttgattccaataaactcaggg

SEQ ID NO: 198 - DNA, 20 bp, synthetic construct
  source 1..20: mol_type = other DNA
gtttgactcacggggatttc

SEQ ID NO: 199 - DNA, 20 bp, synthetic construct
  source 1..20: mol_type = other DNA
ccggtggtgcagatgaactt

SEQ ID NO: 200 - DNA, 35 bp, synthetic construct
  source 1..35: mol_type = other DNA
gatccatgcgtctcacatgttggtggatggcccat

SEQ ID NO: 201 - DNA, 39 bp, synthetic construct
  source 1..39: mol_type = other DNA
catgacgtcgtctcaagccgcctccatggtggtgatggt

SEQ ID NO: 202 - DNA, 45 bp, synthetic construct
  source 1..45: mol_type = other DNA
gctggctagcgtttaaacttaagcttgccaccatgttggtggatg

SEQ ID NO: 203 - DNA, 45 bp, synthetic construct
  source 1..45: mol_type = other DNA
cagcgggtttaaacgggccctctagactagcctccatggtggtga